SEQUENCE LISTING (17 sequences)

SEQ ID NO: 1 - protein, 2432 aa, Semliki Forest virus
MAAKVHVDIEADSPFIKSLQKAFPSFEVESLQVTPNDHANARAFSHLATKLIEQETDKDTLILDIGSAPSRRMMSTHKYHCVCPMRSAEDPERLVCYAKKLAAASEKVLDREIAGKITDLQTVMATPDAESPTFCLHTDVTCRTAAEVAVYQDVYAVHAPTSLYHQAMKGVRTAYWIGFDTTPFMFDALAGAYPTYATNWADEQVLQARNIGLCAASLTEGRLGKLSILRKKQLKPCDTVMFSVGSTLYTESRKLLRSWHLPSVFHLKGKQSFTCRCDTIVSCEGYVVKKITMCPGLYGKTVGYAVTYHAEGFLVCKTTDTVKGERVSFPVCTYVPSTICDQMTGILATDITPEDAQKLLVGLNQRIVVNGRTQRNTNTMKNYLLPIVAVAFSKWAREYKADLDDEKPLGVRERSLTCCCLWAFKTRKMHTMYKKPDTQTIVKVPSEFNSFVIPSLWSTGLAIPVRSRIKMLLAKKTKRESIPVLDASSARDAEQEEKERLEAELTREALPPLVPTAPAETGVVDVDVEELEYHAGAGVVETPRSALKVTAQPNGVLLGNYVVLSPQTVLKSSKLAPVHPLAEQVKIITHNGRAGRYQVDGYDGRVLLPCGSAIPVPEFQALSESATMVYNEREFVNRKLYHIAVHGPSLNTDEENYEKVRAERTDAEYVFDVDKKCCVKREEASGLVLVGELTNPPFHEFAYEGLKIRPSAPYKTTVVGVFGVPGSGKSAIIKSLVTKHDLVTSGKKENCQEIVNDVKKHRGLDIQAKTVDSILLNGCRRAVDILYVDEAFACHSGTLLALIALVKPRSKVVLCGDPKQCGFFNMMQLKVNFNHNICTEVCHKSISRRCTRPVTAIVSTLHYGGKMRTTNPCNKPIIIDTTGQTKPKPGDIVLTCFRGWVKQLQLDYRGHEVMTAAASQGLTRKGVYAVRQKVNENPLYAPASEHVNVLLTRTEDRLVWKTLAGDPWIKVLSNIPQGNFTATLEEWQEEHDKIMKVIEGPAAPVDAFQNKANVCWAKSLVPVLDTAGIRLTAEEWSTIITAFKEDRAYSPVVALNEICTKYYGVDLDSGLFSAPKVSLYYENNHWDNRPGGRMYGFNAATAARLEARHTFLKGQWHTGKQAVIAERKIQPLSVLDNVIPINRRLPHALVTEYKTVKGSRVEWLVNKVRGYHVLLVSEYNLALPRRRVTWLSPLNVTGADRCYDLSLGLPADAGRFDLVFVNIHTEFRIHHYQQCVDHAMKLQMLGGDALRLLKPGGSLLMRAYGYADKISEAVVSSLSRKFSSARVLRPDCVTSNTEVFLLFSNFDNGKRPSTLHQMNTKLSAVYAGEAMHTAGCAPSYRVKRADIATCTEAAVVNAANARGTVGDGVCRAVAKKWPSAFKGEATPVGTIKTVMCGSYPVIHAVAPNFSATTEAEGNRELAAVYRAVAAEVNRLSLSSVAIPLLSTGVFSGGRDRLQQSLNHLFTAMDATDADVTIYCRDKSWEKKIQEAIDTRTAVELLNDDVELTTDLVRVHPDSSLVGRKGYSTTDGSLYSYFEGTKFNQAAIDMAEILTLWPRLQEANEQICLYALGETMDNIRSKCPVNDSDSSTPPRTVPCLCRYAMTAERITRLRSHQVKSMVVCSSFPLPKYHVDGVQKVKCEKVLLFDPTVPSVVSPRKYAASTTDHSDRSLRGFDLDWTTDSSSTASDTMSLPSLQSCDIDSIYEPMAPIVVTADVHPEPAGIADLAADVHPEPADHVDLENPIPPPRPKRAAYLASRAAERPVPAPRKPTPAPRTAFRNKLPLTFGDFDEHEVDALASGITFGDFDDVLRLGRAGAYIFSSDTGSGHLQQKSVRQHNLQCAQLDAVEEEKMYPPKLDTEREKLLLLKMQMHPSEANKSRYQSRKVENMKATVVDRLTSGARLYTGADVGRIPTYAVRYPRPVYSPTVIERFSSPDVAIAACNEYLSRNYPTVASYQITDEYDAYLDMVDGSDSCLDRATFCPAKLRCYPKHHAYHQPTVRSAVPSPFQNTLQSVLAAATKRNCNVTQMRELPTMDSAVFNVECFKRYACSGEYWEEYAKQPIRITTENITTYVTKLKGPKAAALFAKTHNLVPLQEVPMDRFTVDMKRDVKVTPGTKHTEERPKVQVIQAAEPLATAYLCGIHRELVRRLNAVLRPNVHTLFDMSAEDFDAIIASHFHPGDPVLETDIASFDKSQDDSLALTGLMILEDLGVDQYLLDLIEAAFGEISSCHLPTGTRFKFGAMMKSGMFLTLFINTVLNITIASRVLEQRLTDSACAAFIGDDNIVHGVISDKLMAERCASWVNMEVKIIDAVMGEKPPYFCGGFIVFDSVTQTACRVSDPLKRLFKLGKPLTAEDKQDEDRRRALSDEVSKWFRTGLGAELEVALTSRYKVEGCKSILIAMATLARDIKAFKKLRGPVIHLYGGPRLVR

SEQ ID NO: 2 - DNA, 7299 bp, Semliki Forest virus
  source 1..7299: mol_type = genomic DNA
atggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgagaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactggcgaccgacatcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagtcaatacctgttctcgacgcgtcgtcagccagggatgctgaacaagaggagaaggagaggttggaggccgagctgactagagaagccttaccacccctcgtccccaccgcgccggcggagacgggagtcgtcgacgtcgacgttgaagaactagagtatcacgcaggtgcaggggtcgtggaaacacctcgcagcgcgttgaaagtcaccgcacagccgaacggcgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacattgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtgactgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaagggaaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacacgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcacccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagagcgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtataaggtagagggctgcaaaagtatcctcatagccatggccaccttggcgagggacattaaggcgtttaagaaattgagaggacctgttatacacctctacggcggtcctagattggtgcgttaa

SEQ ID NO: 3 - protein, 18 aa, Thosea asigna virus
EGRGSLLTCGDVEENPGP

SEQ ID NO: 4 - DNA, 54 bp, Thosea asigna virus
  source 1..54: mol_type = genomic DNA
gagggaagaggcagcctgctgacctgcggcgacgtggaggagaacccaggccca

SEQ ID NO: 5 - protein, 16 aa, vesiculovirus
MKCLLYLAFLFIGVNC

SEQ ID NO: 6 - DNA, 48 bp, vesiculovirus
  source 1..48: mol_type = genomic DNA
atgaaatgtctcctgtatctcgctttcttgtttatcggtgtcaactgt

SEQ ID NO: 7 - protein, 19 aa, Homo sapiens
MGWSCIILFLVATATGVHS

SEQ ID NO: 8 - DNA, 57 bp, Homo sapiens
  source 1..57: mol_type = genomic DNA
atgggctggtcctgcattattctcttcctcgtagccacggccactggcgttcacagt

SEQ ID NO: 9 - protein, 185 aa, synthetic construct
MARDIDPYKEFGATVELLSFLPSDFFPSVRDLLDTASALYREALESPEHSSPHHTALRQAILSWGELMTLATWVGVNLEDPASRDLVVSYVNTNMGLKFRQLLWFHISSLTFGRETVIEYLVSFGVWIRTPPAYRPPNAPILSTLPETTVVRRRGRSPRRRTPSPRRRRSQSPRRRRSQSRESQS

SEQ ID NO: 10 - DNA, 555 bp, synthetic construct
  source 1..555: mol_type = other DNA
atggcacgcgatattgatccttacaaggagttcggggcaaccgtcgagctgctgagcttcctgccaagcgacttctttccatcagtgcgcgatctgctggataccgctagcgctctgtaccgggaggctctcgagtccccagagcactctagccctcaccacacagcactgaggcaggccattctgtcttggggagagctcatgaccctcgcaacttgggtgggcgtgaacctggaggacccagcatctcgcgacctcgtcgtgtcatacgtgaatacaaatatgggcctgaagtttagacagctgctgtggtttcacatttcaagcctgacattcggacgcgagacagtgattgagtacctggtgtcatttggcgtgtggattagaacccctcccgcttacaggcctcctaatgccccaattctgtctactctgcccgagactaccgtcgtgcgcagacggggaagatctccacgccggcggactccatcacctagacggcgcaggtcccagtctccaagacggcgccggtcacagtctagggagagccagtct

SEQ ID NO: 11 - protein, 569 aa, synthetic construct
MPLSYQHFRRLLLLDDEAGPLEEELPRLADEGLNRRVAEDLNLGNLNVSIPWTHKVGNFTGLASSTVPVFNPHAKTPSFPNIHLHQDIIKKCEQFVGPLTVNEKRRLQLIMPARFYPNVTKYLPLDKGIKPYYPEHLVNHYFQTRHALHTLWKAGILYKRETTHSASFCGSPASWEQKLQDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFAHATPTGWGLVMGHQRMRGTFLASRKYTSFPWLLGCAANWILRGTSFYYVPSALNPYHDPSRGRLGLSRPLLRLPFRPTTGRTSLYADSPSVPSHLPDRVHFASPLHVA

SEQ ID NO: 12 - DNA, 1707 bp, synthetic construct
  source 1..1707: mol_type = other DNA
atgccactgtcttaccagcacttccggcggctgctgctcctcgatgatgaggcaggacccttggaagaggaactaccacggctggccgacgagggactgaatcggcgagtcgccgaggatctcaatctgggaaacctcaatgtctctatcccttggacacacaaggtcggaaattttactggcctcgcttcttcaaccgtgcccgtgtttaacccacacgctaagacccctagctttcctaacattcacctgcaccaggatattattaagaagtgcgagcagttcgtcgggccactgacagtgaacgagaagcgtcgtttacagctgattatgcccgctaggttttaccctaatgtgaccaagtacctgcctctcgataagggaattaagccttactaccccgagcacctggtgaatcactactttcagacacgccacgcactccacacactgtggaaggctggcattctgtacaagagagagactacacactcagcatcattttgcgggtcacccgcttcttgggagcagaagctccaggactggggaccttgcgccgagcacggagagcaccacattcggattccccggactcccgctagggtgaccggcggggtgttcctcgtcgataagaatccacacaatactgccgagtcacggctggtcgtcgattttagccagttttcacgcggaaattaccgcgtctcttggccaaagtttgccgtgcctaacctccagtcactgaccaatctcctgtcatctaatctgagctggctgagcctcgacgtcagcgcagccttctaccacctgccactccacccagcagctatgccccacctgctcgtcggatcatcaggactctcacgttacgtcgctaggctgtcatcaaattctaggatctttaattaccagcacggaaccatgcagaatctccacgattcatgttcaagaaacctctatgtcagcctcctcctgctgtaccagacatttggaagaaagctccacctctactcccaccctattatcctcggctttagaaagatccctatgggagtcgggctgagccctttcctcctggcacagtttactagcgcaggatcactgcctcaggatcacattattcagaagattaaagaatgttttagaaagctgcccgtgaataggccaattgattggaaggtgtgtcagagaatcgtcgggctcctcgggttcgccgccccttttacccagtgcggataccccgctctcatgcccctgtacgcatgtattcagtctaagcaggcttttacttttagccctacatacaaggcatttctgtgtaagcagtacctgaatctctaccccgtcgcccggcagagacccggcctgtgtcaggtgtttgcacacgcaacccctacagggtggggactggtcatgggccaccagcgcatgagagggacatttctggcaagtaggaagtacaccagctttccgtggctgctcgggtgcgccgctaattggatactgcgcggaacctcattttactatgtgcctagcgcactgaacccataccacgacccttcacgggggcggctgggactgtctaggcctctgctccggctgccctttagacctacaactggaagaacttcactgtacgccgatagcccaagcgtcccttcacacctccccgatagagtgcacttcgcctctccactgcacgtcgct

SEQ ID NO: 13 - DNA, 422 bp, synthetic construct
  source 1..422: mol_type = other DNA
acgttagctcgagaagaaggtatattgctgttgacagtgagcgacggacaaacagtgaccaccttagtgaagcttcagatgtaaggtggtcactgtttgtccgctgcctactgcctcggacttcaaggggtcagtcagattttttctcgagaagaaggtatattgctgttgacagtgagcgacgctgaaagtcaatgccccatagtgaagcttcagatgtatggggcattgactttcagcgctgcctactgcctcggacttcaaggggtcagtcagaatttttctcgagaagaaggtatattgctgttgacagtgagcgacgatttgctggcattatatatagtgaagcttcagatgtatatataatgccagcaaatcgctgcctactgcctcggacttcaaggggtcagtcagaatttttt

SEQ ID NO: 14 - protein, 517 aa, vesiculovirus
MLSYLIFALAVSPILGKIEIVFPQHTTGDWKRVPHEYNYCPTSADKNSHGTQTGIPVELTMPKGLTTHQVEGFMCHSALWMTTCDFRWYGPKYITHSIHNEEPTDYQCLEAIKSYKDGVSFNPGFPPQSCGYGTVTDAEAHIVTVTPHSVKVDEYTGEWIDPHFIGGRCKGQICETVHNSTKWFTSSDGESVCSQLFTLVGGIFFSDSEEITSMGLPETGIRSNYFPYISTEGICKMPFCRKQGYKLKNDLWFQIMDPDLDKTVRDLPHIKDCDLSSSIITPGEHATDISLISDVERILDYALCQNTWSKIESGEPITPVDLSYLGPKNPGVGPVFTIINGSLHYFTSKYLRVELESPVIPRMEGKVAGTRIVRQLWDQWFPFGEVEIGPNGVLKTKQGYKFPLHIIGTGEVDSDIKMERVVKHWEHPHIEAAQTFLKKDDTGEVLYYGDTGVSKNPVELVEGWFSGWRSSLMGVLAVIIGFVILMFLIKLIGVLSSLFRPKRRPIYKSDVEMAHFR

SEQ ID NO: 15 - DNA, 1500 bp, synthetic construct
  source 1..1500: mol_type = other DNA
atgctgagctacctgatcttcgccctggccgtgtctcctatcctgggcaagatcgagatcgtgttccctcagcacaccaccggcgactggaaaagagtgccccacgagtacaactactgccccaccagcgccgacaagaatagccacggaacacagacaggcatccccgtggaactgaccatgcctaagggcctgacaacccaccaggtggaaggcttcatgtgtcacagcgccctgtggatgaccacctgtgactttcgttggtacggccccaagtacatcacccacagcatccacaacgaggaacccaccgactaccagtgcctggaagccatcaagagctacaaggacggcgtgtccttcaatcctggattcccacctcagagctgcggctacggcacagtgacagatgccgaggctcacatcgtgaccgtgacacctcacagcgtgaaggtggacgagtacacaggcgagtggatcgaccctcacttcatcggcggcagatgcaagggccagatctgcgagacagtgcacaacagcaccaagtggttcaccagctccgatggcgagagcgtgtgcagccagctgtttaccctcgtcggcggcatcttcttcagcgacagcgaagagatcaccagcatgggcctgcctgaaaccggaatcagaagcaactacttcccctacatcagcaccgagggaatctgcaagatgcccttctgtcggaagcagggctacaagctgaagaacgacctgtggttccagatcatggaccccgacctggataagaccgtgcgggatctgccccacatcaaggactgtgatctgagcagcagcatcatcacccctggcgagcacgccacagacatcagcctgatcagcgacgtggaacggatcctggactacgccctgtgccagaacacctggtctaagatcgagtccggcgagcccatcacacccgtggatctgtcttatctgggccccaagaatcctggcgtgggccctgtgttcaccatcatcaatggcagcctgcactacttcaccagcaagtacctgagagtggaactggaaagccctgtgatccccagaatggaaggcaaggtggccggcacaagaatcgtcagacagctgtgggaccagtggttcccattcggcgaggtggaaatcggccctaacggcgtgctgaaaacaaagcaggggtataagttcccgctgcacatcatcggcaccggcgaagtggacagcgacatcaagatggaacgggtcgtgaagcactgggagcaccctcacattgaggccgctcagaccttcctgaagaaggacgatacaggcgaggtgctgtactacggcgataccggggtgtcaaagaaccccgtcgaactggtggaaggatggtttagcggatggcggtctagcctgatgggagtgctggccgtgatcatcggcttcgtgatcctgatgttcctgattaagctgatcggggtgctgagcagcctgttcaga

SEQ ID NO: 16 - DNA, 15134 bp, synthetic construct
  source 1..15134: mol_type = other DNA
ctagtgcatccaaagaattcaaaaagcttctcgagagtacttctagagcggccgcgcatcgattttccacccgggtggggtaccaggtaagtgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgagaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactggcgaccgacatcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagtcaatacctgttctcgacgcgtcgtcagccagggatgctgaacaagaggagaaggagaggttggaggccgagctgactagagaagccttaccacccctcgtccccaccgcgccggcggagacgggagtcgtcgacgtcgacgttgaagaactagagtatcacgcaggtgcaggggtcgtggaaacacctcgcagcgcgttgaaagtcaccgcacagccgaacggcgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacattgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtgactgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaagggaaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacacgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcacccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagagcgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtataaggtagagggctgcaaaagtatcctcatagccatggccaccttggcgagggacattaaggcgtttaagaaattgagaggacctgttatacacctctacggcggtcctagattggtgcgttaatacacagaattctgattggatccacgatggcacgcgatattgatccttacaaggagttcggggcaaccgtcgagctgctgagcttcctgccaagcgacttctttccatcagtgcgcgatctgctggataccgctagcgctctgtaccgggaggctctcgagtccccagagcactctagccctcaccacacagcactgaggcaggccattctgtcttggggagagctcatgaccctcgcaacttgggtgggcgtgaacctggaggacccagcatctcgcgacctcgtcgtgtcatacgtgaatacaaatatgggcctgaagtttagacagctgctgtggtttcacatttcaagcctgacattcggacgcgagacagtgattgagtacctggtgtcatttggcgtgtggattagaacccctcccgcttacaggcctcctaatgccccaattctgtctactctgcccgagactaccgtcgtgcgcagacggggaagatctccacgccggcggactccatcacctagacggcgcaggtcccagtctccaagacggcgccggtcacagtctagggagagccagtctgagggaagaggcagcctgctgacctgcggcgacgtggaggagaacccaggcccaatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcatgatgacttaataggcacagccttacaagtcaaaatgcccaagagtcacaaggctattcaagcagacggttggatgtgtcatgcttccaaatgggtcactacttgtgacttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaattaattaataatcatattaagggcccaaggggctgcaaaagtatcctcatagccatggccaccttggcgagggacattaaggcgtttaagaaattgagaggacctgttatacacctctacggcggtcctagattggtgcgttaatacacagaattctgattggcgcgccacgatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaacctgcaggaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaa

SEQ ID NO: 17 - DNA, 15130 bp, synthetic construct
  source 1..15130: mol_type = other DNA
ctagtgcatccaaagaattcaaaaagcttctcgagagtacttctagagcggccgcgcatcgattttccacccgggtggggtaccaggtaagtgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgagaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactggcgaccgacatcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagtcaatacctgttctcgacgcgtcgtcagccagggatgctgaacaagaggagaaggagaggttggaggccgagctgactagagaagccttaccacccctcgtccccaccgcgccggcggagacgggagtcgtcgacgtcgacgttgaagaactagagtatcacgcaggtgcaggggtcgtggaaacacctcgcagcgcgttgaaagtcaccgcacagccgaacggcgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacattgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtgactgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaagggaaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacacgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcacccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagagcgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtataaggtagagggctgcaaaagtatcctcatagccatggccaccttggcgagggacattaaggcgtttaagaaattgagaggacctgttatacacctctacggcggtcctagattggtgcgttaatacacagaattctgattggatccacgatggcacgcgatattgatccttacaaggagttcggggcaaccgtcgagctgctgagcttcctgccaagcgacttctttccatcagtgcgcgatctgctggataccgctagcgctctgtaccgggaggctctcgagtccccagagcactctagccctcaccacacagcactgaggcaggccattctgtcttggggagagctcatgaccctcgcaacttgggtgggcgtgaacctggaggacccagcatctcgcgacctcgtcgtgtcatacgtgaatacaaatatgggcctgaagtttagacagctgctgtggtttcacatttcaagcctgacattcggacgcgagacagtgattgagtacctggtgtcatttggcgtgtggattagaacccctcccgcttacaggcctcctaatgccccaattctgtctactctgcccgagactaccgtcgtgcgcagacggggaagatctccacgccggcggactccatcacctagacggcgcaggtcccagtctccaagacggcgccggtcacagtctagggagagccagtctgagggaagaggcagcctgctgacctgcggcgacgtggaggagaacccaggcccaatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcatgatgacttaataggcacagccttacaagtcaaaatgcccaagagtcacaaggctattcaagcagacggttggatgtgtcatgcttccaaatgggtcactacttgtgacttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaattaattaataatcatattaagggcccaaggggctgcaaaagtatcctcatagccatggccaccttggcgagggacattaaggcgtttaagaaattgagaggacctgttatacacctctacggcggtcctagattggtgcgttaatacacagaattctgattggcgcgccacgatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaacctgcaggaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0335531 A1
(43) Pub. Date: Oct. 10, 2024

(54) MULTI-ANTIGEN THERAPEUTIC VACCINES TO TREAT OR PREVENT CHRONIC HEPATITIS B VIRUS INFECTION

(71) Applicant: Carogen Corporation, Farmington, CT (US)

(72) Inventors: Valerian Nakaar, Hamden, CT (US); Bijan Almassian, Cheshire, CT (US); Bhaskara R. Madina, South Windsor, CT (US); Timur O. Yarovinsky, Woodbridge, CT (US); Marie Krady, Hamden, CT (US)

(73) Assignee: Carogen Corporation, Farmington, CT (US)

(21) Appl. No.: 18/755,876

(22) Filed: Jun. 27, 2024

Related U.S. Application Data
(63) Continuation of application No. PCT/US2023/060237, filed on Jan. 6, 2023.
(60) Provisional application No. 63/297,728, filed on Jan. 8, 2022.
(60) Provisional application No. 63/308,103, filed on Feb. 9, 2022.

(51) Int. Cl. A61K 39/29; A61P 31/20; C12N 7/00; C12N 15/86

(52) U.S. Cl. CPC A61K 39/29; A61P 31/20; C12N 7/00; C12N 15/86; C12N 2730/10122; C12N 2730/10123; C12N 2730/10134; C12N 2730/10151; C12N 2750/14143

(57) ABSTRACT

The present invention relates to compositions and methods for therapeutic immunization for treatment of chronic hepatitis B virus (CHB). Methods of the invention include a method generating a high titer hybrid-hepatitis B virus (HBV) vector, methods of treating and/or preventing HBV infection and/or CHB, and methods of inducing a memory T and B cell immune response against HBV infection in a subject administered the VLV composition produced thereby. Furthermore, the invention encompasses a pharmaceutical composition for vaccinating a subject to protect the subject against infection with HBV.

### CROSS-REFERENCE TO RELATED APPLICATIONS

This continuation application claims the benefit of and priority to International Patent Application No. PCT/US2023/060237, filed Jan. 6, 2023, which claims priority to U.S. Provisional Patent Application No. 63/297,728, filed Jan. 8, 2022, and to U.S. Provisional Patent Application No. 63/308,103, filed Feb. 9, 2022, each of which are incorporated by reference in their entireties herein.

### STATEMENT REGARDING FEDERALLY SPONSORED RESEARCH OR DEVELOPMENT

This invention was made with government support under Grant No. 2R44 DK113858 awarded by the National Institute of Diabetes and Digestive and Kidney Diseases (NIDDK), an institute within the National Institutes of Health (NIH). The government has certain rights in the invention.

### STATEMENT REGARDING THE SEQUENCE LISTING

The Sequence Listing associated with this application is provided electronically in.xml format and is hereby incorporated by reference into the specification. The Sequence Listing is provided as a file entitled 25133-100310_sl.xml, created Jan. 5, 2023 which is about 60 KB in size.

### TECHNICAL FIELD

The present invention relates to compositions and methods for therapeutic immunization for treatment of chronic hepatitis B virus (CHB). Methods of the invention include a method generating a high titer hybrid-hepatitis B virus (HBV) vector, methods of treating and/or preventing HBV infection and/or CHB, and methods of inducing a memory T and B cell immune response against HBV infection in a subject administered the VLV composition produced thereby. Furthermore, the invention encompasses a pharmaceutical composition for vaccinating a subject to protect the subject against infection with HBV.

### BACKGROUND

While current antiviral therapies for chronic hepatitis B virus (CHB) infection effectively reduce viremia, they rarely eliminate the virus. Thus, there remains a critical need for new treatment options for this serious disease. Because the human immune system can control HBV but often fails to do so, immunotherapies including therapeutic vaccination represent a promising approach to cure chronic CHB. However, although current HBV vaccine platforms generate potent antibody responses that prevent infection, they typically do not produce the broad CD8 T-cell responses needed to eliminate the virus after infection. Therefore, new vaccine delivery systems that can generate effective therapeutic immune responses to HBV are urgently needed. We have developed an immunotherapy based on our virus like-vesicle (VLV) platform for the treatment of patients with CHB infection.

We have established that an RNA replicon-based vector or VLV carrying RNA encoding one or more of the HBV major antigens [middle surface envelope glycoproteins (MHBs), hepatitis B core antigen (HBcAg), or polymerase] in a single open reading frame (CARG-101) in a polycistronic unit drives a broad multi-specific immune response that produces substantial clearance of HBV in the mouse liver. Treatment of mice chronically infected with adeno-associated virus (AAV)-HBV significantly reduces and, in some animals, eliminates serum HBV surface antigen (HBsAg), a surrogate biomarker for viral persistence in the liver. We have significantly enhanced overall gene expression which led to our next-generation clinical candidate, CARG-201, which induces both T-cell responses and antibodies in comparison to CARG-101. CARG-201 expressing MHBs and HBcAg under separate subgenomic promoters clears serum HBsAg completely in 100% of mice and reduces HBV RNA in the liver to undetectable levels in an AAV mouse model of CHB infection with low antigen burden (HBsAglow). However, in a more stringent AAV-HBV model (HBsAg^{high}), CARG-201 reduces HBsAg levels by only 80%. As high antigen burden is observed in many CHB patients and is associated with T-cell exhaustion or tolerance, successful immunotherapy should improve immunogenicity and overcome T-cell exhaustion and/or tolerance.

Modifications of CARG-201 in any one of, or one or more of three complementary approaches will enhance efficacy and lead to complete clearance of serum HBsAg levels in animals:

First, we have incorporated polymerase (Pol) antigen into CARG-201 to generate CARG-301 (expressing MHBs, HBcAg, plus Pol). A vaccine that generates multi-antigen specific T cells is better positioned to provide the desired therapeutic effect compared to one or two antigens.
Moreover, Pol is a highly immunogenic CD4 and CD8 T-cell target, and because of its high sequence conservation, it may prevent the generation of escape mutants in the T-cell epitope.
Second, we have engineered CARG-201 to incorporate human IgK signal sequence for the polymerase (pol) gene and VSV G glycoprotein signal sequence for the HBc gene. It is known that secreted proteins generally lead to the activation of dendritic cells, the enhancement of HBV antigen presentation, and the generation of new cytotoxic T-cell responses by epitope spreading. In this manner, the quality and quantity of the T-cell responses against HBV antigens may be further enhanced as compared to soluble and non-secreted counterparts. Secreted proteins also contribute to the adaptive immune responses by being taken up by antigen-presenting cells and processed via the major histocompatibility complex (MHC) class II pathway.
Third, we have also targeted for disruption the programmed death-ligand 1 (PD-L1) immune checkpoint by short hairpin RNA (shRNA) to achieve sustained long-term viral suppression or complete elimination of the virus in the liver. Checkpoint inhibition can enhance ex vivo effector T-cell responses from patients with other chronic infections. We predict that disruption of the PD-1/PD-L1 pathway will re-invigorate the otherwise exhausted T-cell function
The combined effects of shRNA-mediated PD-L1 inhibition and the improved secretion of the HBV antigens as result of ER-targeting confer on these modified multivalent constructs a superior therapeutic index necessary to clear the virus and to halt disease progression and mortality in CHB patients.

### SUMMARY

A high titer hybrid-hepatitis B virus (HBV) vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding an HBV antigen or fragment thereof, operably linked to a 2A DNA encoding a 2A peptide, which is in turn operably linked to a vesicular stomatitis virus (VSV) G DNA encoding a VSV G protein, wherein the SFV non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G and T-11978-C, wherein the vector lacks nucleotide sequences which encode SFV structural proteins, further wherein when the vector is propagated in cell culture, titers of at least 10⁷ plaque forming units (pfu) per ml of virus like vesicles (VLVs) are obtained.

In an embodiment, the present disclosure relates to a high-titer hybrid virus vector for treatment, prophylaxis or prevention of hepatitis B virus infections comprising the following operably linked sequence elements:
a) a first DNA sequence comprising a DNA promoter sequence,
b) a second DNA sequence encoding alphavirus non-structural protein polynucleotide sequences,
c) a third DNA sequence encoding at least two alphavirus subgenomic promoters,
d) a fourth DNA sequence comprising at least two sequence domains each independently selected from the group consisting of
   i) a sequence domain encoding an HBV antigenwherein the sequence domain comprises at least one heterologous secretion signal sequence; and
   ii) a sequence domain encoding a human short hairpin RNA (shRNA); and
e) a fifth DNA sequence encoding a vesiculovirus glycoprotein. The sequences may be operably linked in any functional or useful ordering.

In some embodiments, the first DNA sequence comprising a DNA promoter sequence comprises a CMV promoter, optionally including a CMV enhancer. In some embodiments, the promoter and optional enhancer can be any effective promoter/enhancer. In some embodiments, the promoter and optional enhancer can be any construct that recruits RNA polymerase II in eukaryotic cells (preferably mammalian cells).

In some embodiments, the at least two alphavirus subgenomic promoters are synthesized on the negative strand of the RNA that is synthesized by the alphavirus non-structural protein polynucleotide sequences (such as SFVnsp1-4). In some embodiments, the subgenomic promoters are recognized by SFVnsp1-4. In some embodiments, the subgenomic promoters are recognized by the alphavirus non-structural protein to generate 26S subgenomic RNA. In some embodiments, the subgenomic promoters are recognized by SFVnsp1-4 to generate 26S subgenomic RNA.

In further embodiments, the alphavirus non-structural protein polynucleotide sequence is a semiliki forest virus sequence having at least 70% homology to SEQ ID NO: 2.

In further embodiments, the alphavirus non-structural protein polynucleotide sequence is a semiliki forest virus sequence having at least 80% homology to SEQ ID NO: 2.

In further embodiments, the alphavirus non-structural protein polynucleotide sequence is a semiliki forest virus sequence having at least 90% homology to SEQ ID NO: 2.

In further embodiments, the alphavirus non-structural protein polynucleotide sequence is a semiliki forest virus sequence having at least 95% homology to SEQ ID NO: 2.

In further embodiments, the alphavirus non-structural protein polynucleotide sequence is a semiliki forest virus sequence having at least 99% homology to SEQ ID NO: 2.

In further embodiments, the sequence domain encoding the HBV antigen is selected from a hepatitis B core antigen (HBcAg), a hepatitis B surface antigen (HBsAg), polymerase (Pol), and HBx, and combinations thereof.

In further embodiments, the hepatitis B core antigen (HBcAg) is a cysteine-modified HBcAg.

In further embodiments, the cysteine-modified HBcAg comprises a polynucleotide sequence having at least 70% homology to SEQ ID NO: 10.

In further embodiments, wherein the cysteine-modified HBcAg comprises a polynucleotide sequence having at least 80% homology to SEQ ID NO: 10.

In further embodiments, the cysteine-modified HBcAg comprises a polynucleotide sequence having at least 90% homology to SEQ ID NO: 10.

In further embodiments, the cysteine-modified HBcAg comprises a polynucleotide sequence having at least 95% homology to SEQ ID NO: 10.

In further embodiments, the cysteine-modified HBcAg comprises a polynucleotide sequence having at least 99% homology to SEQ ID NO: 10.

In further embodiments, the hepatitis B surface antigen (HBsAg) is selected from middle (M), large (L), and small(S) hepatitis B surface antigens.

In further embodiments, the polymerase (Pol) comprises a truncated and modified polynucleotide sequence.

In further embodiments, the polymerase (Pol) comprises a polynucleotide sequence having at least 70% homology to SEQ ID NO: 12.

In further embodiments, the polymerase (Pol) comprises a polynucleotide sequence having at least 80% homology to SEQ ID NO: 12.

In further embodiments, the polymerase (Pol) comprises a polynucleotide sequence having at least 90% homology to SEQ ID NO: 12.

In further embodiments, the polymerase (Pol) comprises a polynucleotide sequence having at least 95% homology to SEQ ID NO: 12.

In further embodiments, the polymerase (Pol) comprises a polynucleotide sequence having at least 99% homology to SEQ ID NO: 12.

In further embodiments, the heterologous secretion signal sequence is a human IgK secretion signal sequence or a VSV G secretion signal sequence.

In further embodiments, the human IgK secretion signal sequence comprises a polynucleotide sequence having at least 70% homology to SEQ ID NO: 8.

In further embodiments, the human IgK secretion signal sequence comprises a polynucleotide sequence having at least 80% homology to SEQ ID NO: 8.

In further embodiments, the human IgK secretion signal sequence comprises a polynucleotide sequence having at least 90% homology to SEQ ID NO: 8.

In further embodiments, the human IgK secretion signal sequence comprises a polynucleotide sequence having at least 95% homology to SEQ ID NO: 8.

In further embodiments, the human IgK secretion signal sequence comprises a polynucleotide sequence having at least 99% homology to SEQ ID NO: 8.

In further embodiments, the VSV G secretion signal sequence comprises a polynucleotide sequence having at least 70% homology to SEQ ID NO: 6.

In further embodiments, the VSV G secretion signal sequence comprises a polynucleotide sequence having at least 80% homology to SEQ ID NO: 6.

In further embodiments, the VSV G secretion signal sequence comprises a polynucleotide sequence having at least 90% homology to SEQ ID NO: 6.

In further embodiments, the VSV G secretion signal sequence comprises a polynucleotide sequence having at least 95% homology to SEQ ID NO: 6.

In further embodiments, the VSV G secretion signal sequence comprises a polynucleotide sequence having at least 99% homology to SEQ ID NO: 6.

In further embodiments, the sequence domain encoding an HBV antigen is a cysteine-modified hepatitis B core antigen (HBcAg) comprising a polynucleotide sequence having at least 70% homology to SEQ ID NO: 10, and wherein the heterologous secretion signal sequence is a VSV G secretion signal sequence comprising a polynucleotide sequence having at least 70% homology to SEQ ID NO: 6.

In further embodiments, the sequence domain encoding an HBV antigen is a polymerase (Pol) gene comprising a polynucleotide sequence having at least 70% homology to SEQ ID NO: 12, and wherein the heterologous secretion signal sequence is a human IgK secretion signal sequence comprising a polynucleotide sequence having at least 70% homology to SEQ ID NO: 6.

In further embodiments, the sequence domain encoding a human short hairpin RNA (shRNA) targets PD-L1.

In further embodiments, the sequence domain encoding the shRNA comprises a polynucleotide sequence having at least 70% homology to SEQ. ID NO: 13.

In further embodiments, the sequence domain encoding the shRNA comprises a polynucleotide sequence having at least 80% homology to SEQ. ID NO: 13.

In further embodiments, the sequence domain encoding the shRNA comprises a polynucleotide sequence having at least 90% homology to SEQ. ID NO: 13.

In further embodiments, the sequence domain encoding the shRNA comprises a polynucleotide sequence having at least 95% homology to SEQ. ID NO: 13.

In further embodiments, the sequence domain encoding the shRNA comprises a polynucleotide sequence having at least 99% homology to SEQ. ID NO: 13.

In further embodiments, the DNA sequence encoding a vesiculovirus glycoprotein encodes a New Jersey (NJ) serotype vesiculovirus glycoprotein.

In further embodiments, the NJ serotype vesiculovirus glycoprotein comprises a polynucleotide sequence having at least 70% homology to SEQ ID NO: 15.

In further embodiments, the NJ serotype vesiculovirus glycoprotein comprises a polynucleotide sequence having at least 80% homology to SEQ ID NO: 15.

In further embodiments, the NJ serotype vesiculovirus glycoprotein comprises a polynucleotide sequence having at least 90% homology to SEQ ID NO: 15.

In further embodiments, the NJ serotype vesiculovirus glycoprotein comprises a polynucleotide sequence having at least 95% homology to SEQ ID NO: 15.

In further embodiments, the NJ serotype vesiculovirus glycoprotein comprises a polynucleotide sequence having at least 99% homology to SEQ ID NO: 15.

In further embodiments, the sequence domain encoding an HBV antigen is linked to the sequence encoding a vesiculovirus glycoprotein by a sequence comprising 2A ribosome skipping sequence.

In further embodiments, the 2A ribosome skipping sequence is a Thosea asigna virus 2A (T2A) sequence.

In further embodiments, the T2A sequence comprises a polynucleotide sequence having at least 70% homology to SEQ ID NO: 4.

In further embodiments, the T2A sequence comprises a polynucleotide sequence having at least 80% homology to SEQ ID NO: 4.

In further embodiments, the T2A sequence comprises a polynucleotide sequence having at least 90% homology to SEQ ID NO: 4.

In further embodiments, the T2A sequence comprises a polynucleotide sequence having at least 95% homology to SEQ ID NO: 4.

In further embodiments, the T2A sequence comprises a polynucleotide sequence having at least 99% homology to SEQ ID NO: 4.

In an embodiment, the vector (or plasmid) comprises the following operably linked sequence elements:
a) a first DNA sequence comprising a DNA promoter sequence,
b) a second DNA sequence encoding alphavirus non-structural protein polynucleotide sequences and having at least 70% homology to SEQ ID NO: 2;
c) a third DNA sequence encoding at least two alphavirus subgenomic promoters,
d) a fourth DNA sequence comprising at least two sequence domains each independently selected from the group consisting of
   i) a sequence domain encoding an HBV antigen comprising a polynucleotide sequence having at least 70% homology to SEQ ID NO: 10 or SEQ ID NO: 12, wherein the sequence domain comprises at least one heterologous secretion signal sequence having at least 70% homology to SEQ ID NO: 6 or SEQ ID NO: 8; and
   ii) a sequence domain encoding a human short hairpin RNA (shRNA), the sequence domain comprising a polynucleotide sequence having at least 70% homology to SEQ ID NO: 13; and
e) a fifth DNA sequence encoding a vesiculovirus glycoprotein having at least 70% homology to SEQ ID NO: 15, optionally wherein the sequence domain encoding an HBV antigen is linked to the sequence encoding a vesiculovirus glycoprotein by a 2A polynucleotide having at least 70% homology to SEQ ID NO: 4.

In further embodiments, the recited homologies are each at least 90% homology.

In further embodiments, titers of at least 1×1010 plaque forming units (pfu) per mL of virus like vesicles (VLVs) are obtained.

In an embodiment, the present disclosure provides for a high-titer hybrid virus vector for generating virus-like vesicles (VLVs) for treatment, prophylaxis or prevention of hepatitis B virus infections.

In an embodiment, the present disclosure provides for Virus-like vesicles (VLVs) containing replicon RNA generated by a high-titer hybrid-virus vector.

In an embodiment, the present disclosure provides for a composition comprising virus-like vesicles (VLVs) produced by a high-titer hybrid virus vector.

In an embodiment, the vector is a plasmid comprising a polynucleotide sequence having at least 70% homology to SEQ ID NO: 16 or SEQ ID NO: 17.

In an embodiment, the vector is a plasmid comprising a polynucleotide sequence having at least 80% homology to SEQ ID NO: 16 or SEQ ID NO: 17.

In an embodiment, the vector is a plasmid comprising a polynucleotide sequence having at least 90% homology to SEQ ID NO: 16 or SEQ ID NO: 17.

In an embodiment, the vector is a plasmid comprising a polynucleotide sequence having at least 95% homology to SEQ ID NO: 16 or SEQ ID NO: 17.

In an embodiment, the vector is a plasmid comprising a polynucleotide sequence having at least 97% homology to SEQ ID NO: 16 or SEQ ID NO: 17.

In an embodiment, the vector is a plasmid comprising a polynucleotide sequence having at least 98% homology to SEQ ID NO: 16 or SEQ ID NO: 17.

In an embodiment, the vector is a plasmid comprising a polynucleotide sequence having at least 99% homology to SEQ ID NO: 16 or SEQ ID NO: 17.

In an embodiment, the vector is a plasmid comprising a polynucleotide sequence consisting of SEQ ID NO: 16 or SEQ ID NO: 17.

In an embodiment, the vector is a plasmid consisting essentially of the polynucleotide sequence of SEQ ID NO: 16 or SEQ ID NO: 17.

In an embodiment, the vector is a plasmid consisting of the polynucleotide sequence of SEQ ID NO: 16 or SEQ ID NO: 17.

In an embodiment, the present disclosure provides for an isolated plasmid comprising a polynucleotide sequence having at least 70% homology to SEQ ID NO: 16 or SEQ ID NO: 17.

In an embodiment, the present disclosure provides for an isolated plasmid consisting essentially of the polynucleotide sequence of SEQ ID NO: 16 or SEQ ID NO: 17.

In an embodiment, the present disclosure provides for an isolated plasmid consisting of the polynucleotide sequence of SEQ ID NO: 16 or SEQ ID NO: 17.

In an embodiment, the present disclosure provides for a method of treating and preventing hepatitis B virus infections in a mammalian subject, the method comprising administering a therapeutically effective amount of a VLV composition a mammalian subject in need thereof.

In an embodiment, the present disclosure provides for a method of immunizing a mammalian subject against hepatitis B virus infections, the method comprising administering a therapeutically effective amount of a VLV composition to a mammalian subject in need thereof.

In an embodiment, the present disclosure provides for a method of downregulating genes associated with hepatitis B virus infections, the method comprising administering a therapeutically effective amount of a VLV composition to a mammalian subject in need thereof.

In some embodiments, the mammalian subject is a human or animal.

In further embodiments, the present disclosure provides for a use of a VLV composition in the manufacture of a medicament for the treatment, prophylaxis, or prevention of hepatitis B virus infections in a mammalian subject in need thereof.

In further embodiments, the mammalian subject is a human or animal.

In further embodiments, the present disclosure provides for a method of producing virus-like vesicles (VLVs) for treatment, prophylaxis, or prevention of hepatitis B virus infections comprising the steps of:
a) generating a high-titer virus vector comprising at least two alphavirus sub-genomic promoters; and at least two sequence domains each independently selected from the group consisting of sequence domain encoding HBV antigens: a core (HBcAg), surface [middle (M), large (L), and small(S) HBs], polymerase (Pol) and HBx and combinations thereof, wherein the protein nucleotide sequences comprise at least one heterologous secretion signal sequence; and a sequence domain encoding a human short hairpin RNA (shRNA).
b) transfecting BHK-21 or HEK293 T cells with the high-titer virus vector of step (a),
c) incubating the transfected BHK-21 or HEK293 T cells of step (b) in a buffer solution for a suitable time and at a suitable temperature to propagate VLVs; and
d) isolating the VLVs from the BHK-21 or HEK293 T cells and buffer solution by a technique selected from the group consisting of ultrafiltration, centrifugation, tangential flow filtration, affinity purification, ion exchange chromatography, and combinations thereof; wherein the isolating of step (d) yields VLVs of a high titer.

These and other aspects of the present invention will become apparent from the disclosure herein.

### BRIEF DESCRIPTION OF THE DRAWINGS

Aspects and advantages of the present disclosure will become apparent from the following exemplary embodiments taken in conjunction with the accompanying drawings, of which:

FIGS. 1A-1E depict effects of dp-HBc.MHs (CARG-201) and dp-MHs on HBsAg levels in a chronic AAV-HBV model. FIG. 1A depicts an exemplary chematic of single-antigen (dp-MHs) and dual-antigen (CARG-201) vectors, FIG. 1B depicts ELISA analysis of HBsAg (ng/mL), FIG. 1C depicts qRT-PCR of liver HBV RNA, FIG. 1D depicts flow cytometry of HBV-specific CD8⁺ T cells using intracellular staining for IFNγ after stimulation with HBsAg or HBcAg peptide pools, and FIG. 1E depicts ELISPOT of HBV-specific CD8⁺ T cells using an HBsAg peptide pool;

FIG. 2 depicts therapeutic vaccine candidate CARG-201 in prime-boost immunization controls HBV in mice with higher pre-existing HBV antigen levels;

FIGS. 3A-3B depict construction and expression VLV-based recombinant multivalent HBV vaccines. FIG. 3A depicts exemplary schema of CARG-201 and CARG-301candidates, and FIG. 3B depicts expression of HBV genes as assayed by western blot in BHK21 cell lysate;

FIGS. 4A-4C depict therapeutic vaccine candidates CARG-201 and CARG-301 in prime-boost Immunization controls HBV in mice with high pre-existing HBV antigen levels. FIG. 4A depicts average and individual (with average bar) values of HBsAg levels as a function of time, FIG. 4B depicts serum anti-HBS at week 17, and FIG. 4C depicts serum alanine transaminase (ALT) levels as a function of time;

FIG. 5 is an exemplary schematic depiction of a modified CARG-201 vaccine construct for enhanced immunogenicity and efficacy by incorporating secretory signals and shRNA for PD-L1;

FIGS. 6A-6B depict a comparison of the immunogenicity of modified CARG-201 variants in naïve CB6F1 mice. FIG. 6A depicts spleen cellularity at day 7 post immunication;

FIG. 6B depicts the frequency of cytokine producing T cells after polyclonal stimulation; FIG. 6C depicts HBS peptide pool, and FIG. 6D depicts HBC peptide pool;

FIGS. 7A-7B depict expression and secretion of VLV-based recombinant modified CARG-301 multivalent HBV vaccines. FIG. 7A depicts exemplary Schema of CARG-301 candidate constructs, and FIG. 7B depicts expression of HBV genes as assayed by western blot in BHK21 cell lysate;

FIGS. 8A-8C depict shRNA inhibition of PD-L1 expression in stably transfected BHK21 cells in vitro. FIG. 8A depicts exemplary schema of VLV therapeutic vaccines; FIG. 8B depicts a mouse cDNA clone of PD-L1; FIG. 8C depicts VLVs produced by transfecting BHK21 cells using three versions of shRNA 3XT2A constructs and VLV-3×T2A without shRNA;

FIG. 9A-9C depict downregulation of PD-L1 with shRNA VLV constructs. FIG. 9A depicts exemplary empty VLV constructs in which shRNA is driven by one or two sub-genomic promoters, FIG. 9B depicts Western blot analysis of stable BHK21 cells constitutively expressing PD-L1, and FIG. 9C depicts densitometric quantification of blot after normalization to actin;

FIGS. 10A-10B show CARG-201 dramatically reduces serum HBsAg levels and induces core-specific T cells in a more stringent AAV-HBV model HBsAg^{High}. FIG. 10A depicts serum HBsAg levels for mice transduced with AAV-HBV1.2-mer and chronicity was fully established by week 8 (wk8), and mice were then segregated into high antigen (HBsAgHigh) and low antigen (HBsAgLow), FIG. 10B depicts core specific T cells and PD1 cells;

FIGS. 11A-11F depict blockade of PD-1/PD-L1 pathway by shRNA in vivo significantly inhibits expression of immune checkpoints and inhibitory receptors in MC38 tumored-mice. Gene expression is shown for CTLA 4 (FIG. 11A), PD-L1 (FIG. 11B), Tigit (FIG. 11C), PD-1 (FIG. 11D), PD-L2 (FIG. 11E), and Lag3 (FIG. 11F);

FIG. 12 depicts exemplary strategies to improve efficacy of therapeutic HBV vaccine in animals and in humans; and

FIG. 13 depicts exemplary rationales for development of optimized VLV candidate: a paradigm for therapeutic vaccine (immunotherapy) against HBV.

### DETAILED DESCRIPTION

The present disclosure generally relates to compositions and methods for therapeutic immunization for treatment of chronic hepatitis B virus (CHB). Methods of the invention include a method generating a high titer hybrid-hepatitis B virus (HBV) vector, methods of producing related VLVs, methods of treating and/or preventing HBV infection and/or CHB, and methods of inducing a memory T and B cell immune response against HBV infection in a subject administered the VLV composition produced thereby.

HBV: Significance of the Problem. HBV infection is a major global public health problem. Worldwide, approximately 2 billion people are infected with hepatitis B virus (HBV) during their lifetime, and >240 million have current HBV infection, and about 600,000 people die from HBV-related liver disease every year. Patients with chronic HBV (CHB) infection, including inactive carriers of HBV, have an increased risk of developing liver cirrhosis, hepatic failure, and hepatocellular carcinoma (HCC). Although most of these patients will not develop HBV-related complications. 15-40% will develop serious complications during their lifetime. CHB has various clinical stages defined by HBV DNA titer, presence of hepatitis B e antigen (HBeAg, a secreted form of the core protein) and the presence or absence of liver inflammation measured by liver transaminase levels. CHB infection occurs as a result of continuous interaction between the viral replication and immune responses. T cells are exhausted by the persistent antigen exposure, which contribute to the persistence of HBV infection. When T cells encounter HBV antigens presented by the intrahepatic antigen presenting cells (APCs), such as the dendritic cells (DCs) and Kupffer cells, the costimulatory signals received by T cells are very weak. This result in immune tolerance rather than functional activation. In addition, the immunosuppressive microenvironment is formed in the liver of patients with CHB with high proportion of regulatory T cells (Tregs) and myeloid-derived suppressor cells (MDSCs). These provide T cells with inhibitory signals and disturb T cell-mediated anti-HBV functions.

Limitations of the current HBV vaccine. Despite its success in preventing HBV infection, the current HBV vaccine (recombinant HBsAg adsorbed to alum) has a number of characteristics that are suboptimal. First, it does not induce a protective antibody response in all immunized individuals. Second, between two and four doses of the vaccine are recommended to induce long-lasting immunity. This need for repeated immunization makes the vaccine somewhat challenging to administer in many regions of the world, especially those lacking the appropriate medical infrastructure. Third, the protective antibody response wancs after immunization, and declines to below protective levels (>10 IU/L) in up to 60% of vaccinated individuals. Fourth, escape mutations in the surface protein gene can produce virus that is resistant to the antibody response generated by the vaccine Finally, as discussed above, although it clicits a protective antibody response that prevents infection, the current vaccine does not generate a strong CD8 T cell response, and it has not been effective in clinical trials to control virus replication in those who are already infected with HBV.

Current therapies for CHB. Current standard of care for CHB includes anti-viral and immune-enhancing drugs, such as tenofovir, entecavir and PEGylated IFN which are very effective at slowing down disease, are curative only 8-12% of the patients treated. Cessation of antiviral therapy is often accompanied by a rebound in the viral load; therefore, lifelong treatment is required. Although available antiviral drugs can lead to suppression of serum HBV viral load to undetectable levels efficiently, however, they usually fail to achieve sero-clearance of hepatitis B surface antigen (HBsAg), which indicates eradication of HBV infection, the ultimate goal of antiviral treatment for CHB. The failure to achieve HBsAg sero-clearance may be duc to emerging drug-resistant HBV variants and the covalently closed circular DNA (cccDNA) in remaining infected hepatocytes. As none of these clinical therapies achieve long-term virological control in majority of patients with CHB, therefore, there is an urgent need to develop new therapies to improve HBsAg clearance and virological curc.

New Modalities for HBV Treatment. The failure of HBsAg sero-clearance requires the development of novel therapeutic strategies for achieving durable viral remission. One strategy is to target virus directly, by targeting viral entry, viral assembly/encapsidation, preS1 or hepatitis B surface antigen (HBsAg) secretion, envelopment and cccDNA. Another strategy is to interfere with the host mechanisms, by using Toll-like receptor (TLR) agonists, cytokines and the blocking of PD-1/PD-L1. In addition, therapeutic vaccines based on recombinant HBV proteins or HBV-envelope subviral particles, DNA and T-cell peptide epitope resent another promising strategy for HBV eradication. Therapeutic vaccination is aimed at eliminating persistent viral infection by augmenting the patient's immune responses. Individuals who become acutely infected but ultimately clear the virus have a relatively strong, multi-specific T-cell response to HBV. However, in those who become chronically infected, the T-cell response is much weaker in magnitude and is directed toward fewer viral antigens. This suboptimal immune response persists in chronically infected individuals despite the continual presence of viral antigens in the liver and blood. Although the current HBV vaccines induce potent antibody responses that prevent infection, they do not elicit the virus-specific T cells needed to control an established infection. New technologies that generate an effective T cell-dependent immune response to HBV are urgently needed. One promising approach for treating CHB is a therapeutic vaccine capable of inducing virus-specific CD8 T cells to clear HBV infection.

Functional cure of HBV. The ultimate goal of HBV treatment is ‘functional cure’. According to the meeting of AASLD and EASL, functional cure is defined as a sustained loss of HBsAg in scrum. In this scenario, although HBV cccDNA remains at low levels, a functional adaptive immune response ensures suppression of viral replication without treatment, analogous to that which occurs in clearance of acute HBV. A strong HBV-specific CD8 T cell response is required for HBV clearance in acute infection, but in CHB the T cell response is dysfunctional and is not fully restored by NUCs. As functional cure is rarely achieved with current therapy, alternative treatments that can be given in shorter and finite courses are urgently required. CHB infection is the result of complex interactions between HBV and the host, and an impaired immune response to viral antigens is believed to be a key factor associated with the CHB carrier state. If this state of immune tolerance could be overcome, the loss of HBeAg or HBsAg from the serum (seroclearance) and sustained control of the HBV infection would be achieved.

Scientific Premise. Current standard-of-care therapies only rarely lead to a functional cure, characterized by sustained loss of HBsAg (with or without HBsAg antibody seroconversion). The goal for the next generation CHB therapies is to achieve a higher rate of functional cure with finite treatment duration. To address this urgent need, we developed targeted shRNA therapeutics for CHB based on VLV delivery platform. The shRNA can be developed as a stand-alone treatment or in combination with therapeutic vaccine to achieve a functional cure. Since the human immune system can control HBV but often fails to do so, immunotherapies including therapeutic vaccine represent a promising approach to cure CHB. However, the therapeutic immune responses generated in the persistent HBV infection are often weak due to CD8 T cell exhaustion. Exhaustion of virus-specific T cells may play an important role in HBV persistence. The interaction between programmed death-1 (PD-1) receptor on lymphocytes and its ligand PD-L1 plays a critical role in T-cell exhaustion by inducing T-cell inactivation indicating that the PD-1/PD-L1 pathway is a good therapeutic candidate for chronic HBV infection. Woodchucks infected with woodchuck hepatitis virus (WHV) can have increased hepatic expression of PD-1-ligand-1 (PD-L1), increased PD-1 on CD8+ T cells, and a limited number of virus-specific T cells. Others have shown that in these animals, combination therapy with aPD-L1 and entecavir (ETV) improved control of viremia and antigenemia compared to ETV treatment alone. In addition, others have shown that PD-L1 blockade synergistically augments HBV-specific CD4 T cells. Furthermore, there is accumulating evidence that immune checkpoint inhibitors can enhance ex vivo effector T-cell responses from patients with other chronic viral, bacterial, or parasitic infection, including HIV, tuberculosis, and malaria. We have found that therapeutic shRNA intervention targeting exhausted T cells by blocking these suppressive pathways can restore the function of these impaired T cells and lead to a functional curc.

### Platform and HBV Immunotherapy

We have found that alphavirus replicons are excellent vaccine vectors because they are highly immunogenic and target dendritic cells. The virus-like vesicles (VLV) vaccine platform is a capsid-free, self-replicating, antigen expression system that represents an attractive alternative to other virus-based vectors. VLV encodes a Semliki Forest virus (SFV) replicon and an additional structural protein, the vesicular stomatitis virus glycoprotein (VSV-G). Following in vitro evolution by 50 passages in BHK-21 cells that led to 10 amino acid changes in SFV nsP1-4, the evolved SFV nonstructural proteins promote high-titer VLV replication in cell culture through increased efficiency of VLV release. VSV-G expression allows for robust and pantropic infectivity, as infectious vesicles composed of SFV replication spherules derived from bulb-shaped plasma membrane invaginations are coated with VSV-G protein and bud from infected cells, spread to uninfected cells, and undergo multiple rounds of infection. VLV are nonpathogenic in mice and rhesus macaques, have little risk of genome integration or reversion to pathogenesis, and are immunogenic in the absence of adjuvant. Recent improvements to the system allow the generation of high-titers of VLV particles as well as high gene expression until multiple subgenomic promoters. Although VLVs mimic the immune stimulating properties of viral vectors, they are safe and non-pathogenic when administered to mice or rhesus macaques, nor do they display neurovirulence when injected directly into mouse brain. These vectors are significant because of their potency, case of high-titer particle production, and predicted safety due to the lack of viral structural proteins. Furthermore, VLVs have a demonstrated large capacity to deliver nucleic acids for the expression of several antigens resulting in induction of T cell and antibody responses against multiple epitopes of multiple antigens and thus help to maximize the potential efficacy of the proposed immunotherapy in patients.
During the last 20 years multiple studies have assessed therapeutic vaccine candidates for CHB therapy using lipopeptide epitope-based vaccine; DNA-based vaccines and adenoviral vectored vaccines. Thus far, attempts at therapeutic vaccination for HBV have been ineffective in reliably inducing functional cure in people with CHB. The persistence of HBV-specific T cell hypo-responsiveness and high baseline HBsAg load of participants, together with limited T cell immunogenicity of the vaccine candidates themselves, are possible reasons that may have hampered the success of these vaccine candidates. Other possibilities for failure include the use of HBsAg which in healthy induces HBs antibodies which block viral entry and prevent infection, the effects on T cell induction and immune restoration in the chronic setting using protein vaccines alone are likely to be minimal. Large proportion of patients had HBeAg+disease, associated with high HBV antigen levels. High antigen loads have been proposed as a barrier to the successful rescue of tolerized T cells by therapeutic vaccination.

Vectors of the present invention may generally be a plasmid or other vector encoding VLVs. The term “vector” is therefore inclusive of plasmids. The plasmids can generally comprise any required elements for VLV production. The vectors or plasmids can be defined by one or more sequence domains or components, or by one or more sequences. Generally, unless clear from the context, plasmids may comprise additional sequence domains or components as necessary or desirable. Sequences may be defined as polynucleotide sequences or corresponding amino acid sequences. Some sequence components (such as shRNAs) may not have corresponding amino acid sequences. Exemplary sequence domains are provided in Table 1 below:

TABLE 1
SEQ ID NOs.

| SEQ | | |
|---|---|---|
| ID NO: | Name | Sequence |
| 1 | SFV nsp1- | MAAKVHVDIEADSPFIKSLQKAFPSFEVESLQVTPNDHANA |
| | 4 Replicon | RAFSHLATKLIEQETDKDTLILDIGSAPSRRMMSTHKYHCV |
| | amino acid | CPMRSAEDPERLVCYAKKLAAASEKVLDREIAGKITDLQT |
| | sequence | VMATPDAESPTFCLHTDVTCRTAAEVAVYQDVYAVHAPT |
| | | SLYHQAMKGVRTAYWIGFDTTPFMFDALAGAYPTYATNW |
| | | ADEQVLQARNIGLCAASLTEGRLGKLSILRKKQLKPCDTV |
| | | MFSVGSTLYTESRKLLRSWHLPSVFHLKGKQSFTCRCDTIV |
| | | SCEGYVVKKITMCPGLYGKTVGYAVTYHAEGFLVCKTTD |
| | | TVKGERVSFPVCTYVPSTICDQMTGILATDITPEDAQKLLV |
| | | GLNQRIVVNGRTQRNTNTMKNYLLPIVAVAFSKWAREYK |
| | | ADLDDEKPLGVRERSLTCCCLWAFKTRKMHTMYKKPDTQ |
| | | TIVKVPSEFNSFVIPSLWSTGLAIPVRSRIKMLLAKKTKRESI |
| | | PVLDASSARDAEQEEKERLEAELTREALPPLVPTAPAETGV |
| | | VDVDVEELEYHAGAGVVETPRSALKVTAQPNGVLLGNYV |
| | | VLSPQTVLKSSKLAPVHPLAEQVKIITHNGRAGRYQVDGY |
| | | DGRVLLPCGSAIPVPEFQALSESATMVYNEREFVNRKLYHI |
| | | AVHGPSLNTDEENYEKVRAERTDAEYVFDVDKKCCVKRE |
| | | EASGLVLVGELTNPPFHEFAYEGLKIRPSAPYKTTVVGVFG |
| | | VPGSGKSAIIKSLVTKHDLVTSGKKENCQEIVNDVKKHRGL |
| | | DIQAKTVDSILLNGCRRAVDILYVDEAFACHSGTLLALIAL |
| | | VKPRSKVVLCGDPKQCGFFNMMQLKVNFNHNICTEVCHK |
| | | SISRRCTRPVTAIVSTLHYGGKMRTTNPCNKPIIIDTTGQTKP |
| | | KPGDIVLTCFRGWVKQLQLDYRGHEVMTAAASQGLTRKG |
| | | VYAVRQKVNENPLYAPASEHVNVLLTRTEDRLVWKTLAG |
| | | DPWIKVLSNIPQGNFTATLEEWQEEHDKIMKVIEGPAAPVD |
| | | AFQNKANVCWAKSLVPVLDTAGIRLTAEEWSTIITAFKEDR |
| | | AYSPVVALNEICTKYYGVDLDSGLFSAPKVSLYYENNHWD |
| | | NRPGGRMYGFNAATAARLEARHTFLKGQWHTGKQAVIAE |
| | | RKIQPLSVLDNVIPINRRLPHALVTEYKTVKGSRVEWLVNK |
| | | VRGYHVLLVSEYNLALPRRRVTWLSPLNVTGADRCYDLSL |
| | | GLPADAGRFDLVFVNIHTEFRIHHYQQCVDHAMKLQMLG |
| | | GDALRLLKPGGSLLMRAYGYADKISEAVVSSLSRKFSSAR |
| | | VLRPDCVTSNTEVFLLFSNFDNGKRPSTLHQMNTKLSAVY |
| | | AGEAMHTAGCAPSYRVKRADIATCTEAAVVNAANARGTV |
| | | GDGVCRAVAKKWPSAFKGEATPVGTIKTVMCGSYPVIHA |
| | | VAPNFSATTEAEGNRELAAVYRAVAAEVNRLSLSSVAIPLL |
| | | STGVFSGGRDRLQQSLNHLFTAMDATDADVTIYCRDKSWE |
| | | KKIQEAIDTRTAVELLNDDVELTTDLVRVHPDSSLVGRKGY |
| | | STTDGSLYSYFEGTKFNQAAIDMAEILTLWPRLQEANEQIC |
| | | LYALGETMDNIRSKCPVNDSDSSTPPRTVPCLCRYAMTAER |
| | | ITRLRSHQVKSMVVCSSFPLPKYHVDGVQKVKCEKVLLFD |
| | | PTVPSVVSPRKYAASTTDHSDRSLRGFDLDWTTDSSSTASD |
| | | TMSLPSLQSCDIDSIYEPMAPIVVTADVHPEPAGIADLAADV |
| | | HPEPADHVDLENPIPPPRPKRAAYLASRAAERPVPAPRKPTP |
| | | APRTAFRNKLPLTFGDFDEHEVDALASGITFGDFDDVLRLG |
| | | RAGAYIFSSDTGSGHLQQKSVRQHNLQCAQLDAVEEEKM |
| | | YPPKLDTEREKLLLLKMQMHPSEANKSRYQSRKVENMKA |
| | | TVVDRLTSGARLYTGADVGRIPTYAVRYPRPVYSPTVIERF |
| | | SSPDVAIAACNEYLSRNYPTVASYQITDEYDAYLDMVDGS |
| | | DSCLDRATFCPAKLRCYPKHHAYHQPTVRSAVPSPFQNTL |
| | | QSVLAAATKRNCNVTQMRELPTMDSAVENVECFKRYACS |
| | | GEYWEEYAKQPIRITTENITTYVTKLKGPKAAALFAKTHNL |
| | | VPLQEVPMDRFTVDMKRDVKVTPGTKHTEERPKVQVIQA |
| | | AEPLATAYLCGIHRELVRRLNAVLRPNVHTLFDMSAEDFD |
| | | AIIASHFHPGDPVLETDIASFDKSQDDSLALTGLMILEDLGV |
| | | DQYLLDLIEAAFGEISSCHLPTGTRFKFGAMMKSGMFLTLFI |
| | | NTVLNITIASRVLEQRLTDSACAAFIGDDNIVHGVISDKLMA |
| | | ERCASWVNMEVKIIDAVMGEKPPYFCGGFIVFDSVTQTAC |
| | | RVSDPLKRLFKLGKPLTAEDKQDEDRRRALSDEVSKWFRT |
| | | GLGAELEVALTSRYKVEGCKSILIAMATLARDIKAFKKLRG |
| | | PVIHLYGGPRLVR |
| 2 | SFV nsp1- | atggccgccaaagtgcatgttgatattgaggctgacagcccattcatcaagtctttgcagaagg |
| | 4 Replicon | catttccgtcgttcgaggtggagtcattgcaggtcacaccaaatgaccatgcaaatgccagag |
| | DNA | cattttcgcacctggctaccaaattgatcgagcaggagactgacaaagacacactcatcttgga |
| | sequence | tatcggcagtgcgccttccaggagaatgatgtctacgcacaaataccactgcgtatgccctatg |
| | | cgcagcgcagaagaccccgaaaggctcgtatgctacgcaaagaaactggcagcggcctcc |
| | | gagaaggtgctggatagagagatcgcaggaaaaatcaccgacctgcagaccgtcatggcta |
| | | cgccagacgctgaatctcctaccttttgcctgcatacagacgtcacgtgtcgtacggcagccg |
| | | aagtggccgtataccaggacgtgtatgctgtacatgcaccaacatcgctgtaccatcaggcga |
| | | tgaaaggtgtcagaacggcgtattggattgggtttgacaccaccccgtttatgtttgacgcgcta |
| | | gcaggcgcgtatccaacctacgccacaaactgggccgacgagcaggtgttacaggccagg |
| | | aacataggactgtgtgcagcatccttgactgagggaagactcggcaaactgtccattctccgc |
| | | aagaagcaattgaaaccttgcgacacagtcatgttctcggtaggatctacattgtacactgaga |
| | | gcagaaagctactgaggagctggcacttaccctccgtattccacctgaaaggtaaacaatcctt |
| | | tacctgtaggtgcgataccatcgtatcatgtgaagggtacgtagttaagaaaatcactatgtgcc |
| | | ccggcctgtacggtaaaacggtagggtacgccgtgacgtatcacgcggagggattcctagtg |
| | | tgcaagaccacagacactgtcaaaggagaaagagtctcattccctgtatgcacctacgtcccc |
| | | tcaaccatctgtgatcaaatgactggcatactggcgaccgacatcacaccggaggacgcaca |
| | | gaagttgttagtgggattgaatcagaggatagttgtgaacggaagaacacagcgaaacacta |
| | | acacgatgaagaactatctgcttccgattgtggccgtcgcatttagcaagtgggcgagggaat |
| | | acaaggcagaccttgatgatgaaaaacctctgggtgtccgagagaggtcacttacttgctgct |
| | | gcttgtgggcatttaaaacgaggaagatgcacaccatgtacaagaaaccagacacccagaca |
| | | atagtgaaggtgccttcagagtttaactcgttcgtcatcccgagcctatggtctacaggcctcgc |
| | | aatcccagtcagatcacgcattaagatgcttttggccaagaagaccaagcgagagtcaatacc |
| | | tgttctcgacgcgtcgtcagccagggatgctgaacaagaggagaaggagaggttggaggcc |
| | | gagctgactagagaagccttaccacccctcgtccccaccgcgccggcggagacgggagtc |
| | | gtcgacgtcgacgttgaagaactagagtatcacgcaggtgcaggggtcgtggaaacacctc |
| | | gcagcgcgttgaaagtcaccgcacagccgaacggcgtactactaggaaattacgtagttctgt |
| | | ccccgcagaccgtgctcaagagctccaagttggcccccgtgcaccctctagcagagcaggt |
| | | gaaaataataacacataacgggagggccggccgttaccaggtcgacggatatgacggcag |
| | | ggtcctactaccatgtggatcggccattccggtccctgagtttcaagctttgagcgagagcgcc |
| | | actatggtgtacaacgaaagggagttcgtcaacaggaaactataccatattgccgttcacgga |
| | | ccgtcgctgaacaccgacgaggagaactacgagaaagtcagagctgaaagaactgacgcc |
| | | gagtacgtgttcgacgtagataaaaaatgctgcgtcaagagagaggaagcgtcgggtttggt |
| | | gttggtgggagagctaaccaaccccccgttccatgaattcgcctacgaagggctgaagatca |
| | | ggccgtcggcaccatataagactacagtagtaggagtctttggggttccgggatcaggcaagt |
| | | ctgctattattaagagcctcgtgaccaaacacgatctggtcaccagcggcaagaaggagaact |
| | | gccaggaaatagtcaacgacgtgaagaagcaccgcggactggacatccaggcaaaaacag |
| | | tggactccatcctgctaaacgggtgtcgtcgtgccgtggacatcctatatgtggacgaggcttt |
| | | cgcttgccattccggtactctgctagccctaattgctcttgttaaacctcggagcaaagtggtgtt |
| | | atgcggagaccccaagcaatgcggattcttcaatatgatgcagcttaaggtgaacttcaacca |
| | | caacatctgcactgaagtatgtcataaaagtatatccagacgttgcacgcgtccagtcacggcc |
| | | atcgtgtctacattgcactacggaggcaagatgcgcacgaccaacccgtgcaacaaacccat |
| | | aatcatagacaccacaggacagaccaagcccaagccaggagacatcgtgttaacatgcttcc |
| | | gaggctgggtaaagcagctgcagttggactaccgtggacacgaagtcatgacagcagcagc |
| | | atctcagggcctcacccgcaaaggggtatacgccgtaaggcagaaggtgaatgaaaatccct |
| | | tgtatgcccctgcgtcggagcacgtgaatgtactgctgacgcgcactgaggataggctggtgt |
| | | ggaaaacgctggccggcgatccctggattaaggtcctatcaaacattccacagggtaacttta |
| | | cggccacattggaagaatggcaagaagaacacgacaaaataatgaaggtgattgaaggacc |
| | | ggctgcgcctgtggacgcgttccagaacaaagcgaacgtgtgttgggcgaaaagcctggtg |
| | | cctgtcctggacactgccggaatcagattgacagcagaggagtggagcaccataattacagc |
| | | atttaaggaggacagagcttactctccagtggtggccttgaatgaaatttgcaccaagtactatg |
| | | gagttgacctggacagtggcctgttttctgccccgaaggtgtccctgtattacgagaacaacca |
| | | ctgggataacagacctggtggaaggatgtatggattcaatgccgcaacagctgccaggctgg |
| | | aagctagacataccttcctgaaggggcagtggcatacgggcaagcaggcagttatcgcaga |
| | | aagaaaaatccaaccgctttctgtgctggacaatgtaattcctatcaaccgcaggctgccgcac |
| | | gccctggtgactgagtacaagacggttaaaggcagtagggttgagtggctggtcaataaagt |
| | | aagagggtaccacgtcctgctggtgagtgagtacaacctggctttgcctcgacgcagggtca |
| | | cttggttgtcaccgctgaatgtcacaggcgccgataggtgctacgacctaagtttaggactgcc |
| | | ggctgacgccggcaggttcgacttggtctttgtgaacattcacacggaattcagaatccaccac |
| | | taccagcagtgtgtcgaccacgccatgaagctgcagatgcttgggggagatgcgctacgact |
| | | gctaaaacccggcggcagcctcttgatgagagcttacggatacgccgataaaatcagcgaag |
| | | ccgttgtttcctccttaagcagaaagttctcgtctgcaagagtgttgcgcccggattgtgtcacc |
| | | agcaatacagaagtgttcttgctgttctccaactttgacaacggaaagagaccctctacgctac |
| | | accagatgaataccaagctgagtgccgtgtatgccggagaagccatgcacacggccgggtg |
| | | tgcaccatcctacagagttaagagagcagacatagccacgtgcacagaagcggctgtggtta |
| | | acgcagctaacgcccgtggaactgtaggggatggcgtatgcagggccgtggcgaagaaat |
| | | ggccgtcagcctttaagggagaagcaacaccagtgggcacaattaaaacagtcatgtgcgg |
| | | ctcgtaccccgtcatccacgctgtagcgcctaatttctctgccacgactgaagcggaagggaa |
| | | ccgcgaattggccgctgtctaccgggcagtggccgccgaagtaaacagactgtcactgagc |
| | | agcgtagccatcccgctgctgtccacaggagtgttcagcggcggaagagataggctgcagc |
| | | aatccctcaaccatctattcacagcaatggacgccacggacgctgacgtgaccatctactgca |
| | | gagacaaaagttgggagaagaaaatccaggaagccatagacacgaggacggctgtggagt |
| | | tgctcaatgatgacgtggagctgaccacagacttggtgagagtgcacccggacagcagcctg |
| | | gtgggtcgtaagggctacagtaccactgacgggtcgctgtactcgtactttgaaggtacgaaa |
| | | ttcaaccaggctgctattgatatggcagagatactgacgttgtggcccagactgcaagaggca |
| | | aacgaacagatatgcctatacgcgctgggcgaaacaatggacaacatcagatccaaatgtcc |
| | | ggtgaacgattccgattcatcaacacctcccaggacagtgccctgcctgtgccgctacgcaat |
| | | gacagcagaacggatcacccgccttaggtcacaccaagttaaaagcatggtggtttgctcatc |
| | | ttttcccctcccgaaataccatgtagatggggtgcagaaggtaaagtgcgagaaggttctcctg |
| | | ttcgacccgacggtaccttcagtggttagtccgcggaagtatgccgcatctacgacggaccac |
| | | tcagatcggtcgttacgagggtttgacttggactggaccaccgactcgtcttccactgccagcg |
| | | ataccatgtcgctacccagtttgcagtcgtgtgacatcgactcgatctacgagccaatggctcc |
| | | catagtagtgacggctgacgtacaccctgaacccgcaggcatcgcggacctggcggcagat |
| | | gtgcatcctgaacccgcagaccatgtggacctcgagaacccgattcctccaccgcgcccgaa |
| | | gagagctgcataccttgcctcccgcgcggcggagcgaccggtgccggcgccgagaaagc |
| | | cgacgcctgccccaaggactgcgtttaggaacaagctgcctttgacgttcggcgactttgacg |
| | | agcacgaggtcgatgcgttggcctccgggattactttcggagacttcgacgacgtcctgcgac |
| | | taggccgcgcgggtgcatatattttctcctcggacactggcagcggacatttacaacaaaaatc |
| | | cgttaggcagcacaatctccagtgcgcacaactggatgcggtcgaggaggagaaaatgtac |
| | | ccgccaaaattggatactgagagggagaagctgttgctgctgaaaatgcagatgcacccatc |
| | | ggaggctaataagagtcgataccagtctcgcaaagtggagaacatgaaagccacggtggtg |
| | | gacaggctcacatcgggggccagattgtacacgggagcggacgtaggccgcataccaaca |
| | | tacgcggttcggtacccccgccccgtgtactcccctaccgtgatcgaaagattctcaagcccc |
| | | gatgtagcaatcgcagcgtgcaacgaatacctatccagaaattacccaacagtggcgtcgtac |
| | | cagataacagatgaatacgacgcatacttggacatggttgacgggtcggatagttgcttggac |
| | | agagcgacattctgcccggcgaagctccggtgctacccgaaacatcatgcgtaccaccagc |
| | | cgactgtacgcagtgccgtcccgtcaccctttcagaacacactacagagcgtgctagcggcc |
| | | gccaccaagagaaactgcaacgtcacgcaaatgcgagaactacccaccatggactcggca |
| | | gtgttcaacgtggagtgcttcaagcgctatgcctgctccggagaatattgggaagaatatgcta |
| | | aacaacctatccggataaccactgagaacatcactacctatgtgaccaaattgaaaggcccga |
| | | aagctgctgccttgttcgctaagacccacaacttggttccgctgcaggaggttcccatggaca |
| | | gattcacggtcgacatgaaacgagatgtcaaagtcactccagggacgaaacacacagagga |
| | | aagacccaaagtccaggtaattcaagcagcggagccattggcgaccgcttacctgtgcggca |
| | | tccacagggaattagtaaggagactaaatgctgtgttacgccctaacgtgcacacattgtttgat |
| | | atgtcggccgaagactttgacgcgatcatcgcctctcacttccacccaggagacccggttcta |
| | | gagacggacattgcatcattcgacaaaagccaggacgactccttggctcttacaggtttaatga |
| | | tcctcgaagatctaggggtggatcagtacctgctggacttgatcgaggcagcctttggggaaa |
| | | tatccagctgtcacctaccaactggcacgcgcttcaagttcggagctatgatgaaatcgggcat |
| | | gtttctgactttgtttattaacactgttttgaacatcaccatagcaagcagggtactggagcagag |
| | | actcactgactccgcctgtgcggccttcatcggcgacgacaacatcgttcacggagtgatctc |
| | | cgacaagctgatggcggagaggtgcgcgtcgtgggtcaacatggaggtgaagatcattgac |
| | | gctgtcatgggcgaaaaacccccatatttttgtgggggattcatagtttttgacagcgtcacaca |
| | | gaccgcctgccgtgtttcagacccacttaagcgcctgttcaagttgggtaagccgctaacagct |
| | | gaagacaagcaggacgaagacaggcgacgagcactgagtgacgaggttagcaagtggttc |
| | | cggacaggcttgggggccgaactggaggtggcactaacatctaggtataaggtagagggct |
| | | gcaaaagtatcctcatagccatggccaccttggcgagggacattaaggcgtttaagaaattga |
| | | gaggacctgttatacacctctacggcggtcctagattggtgcgttaa |
| 3 | T2A | EGRGSLLTCGDVEENPGP |
| | Protein | |
| | Sequence | |
| 4 | T2A DNA | gagggaagaggcagcctgctgacctgcggcgacgtggaggagaacccaggccca |
| | Sequence | |
| 5 | VSV G | MKCLLYLAFLFIGVNC |
| | secretion | |
| | signal | |
| | sequence | |
| | (amino | |
| | acid) | |
| 6 | VSV G | atgaaatgtctcctgtatctcgctttcttgtttatcggtgtcaactgt |
| | secretion | |
| | signal | |
| | sequence | |
| | (DNA) | |
| 7 | Human IgK | MGWSCIILFLVATATGVHS |
| | secretion | |
| | signal | |
| | protein | |
| | sequence | |
| | (amino | |
| | acid) | |
| 8 | Human IgK | atgggctggtcctgcattattctcttcctcgtagccacggccactggcgttcacagt |
| | secretion | |
| | signal | |
| | DNA | |
| | sequence | |
| 9 | Cysteine- | MARDIDPYKEFGATVELLSFLPSDFFPSVRDLLDTASALYR |
| | Modified | EALESPEHSSPHHTALRQAILSWGELMTLATWVGVNLEDP |
| | HBc | ASRDLVVSYVNTNMGLKFRQLLWFHISSLTFGRETVIEYLV |
| | Amino acid | SFGVWIRTPPAYRPPNAPILSTLPETTVVRRRGRSPRRRTPSP |
| | sequence | RRRRSQSPRRRRSQSRESQS |
| 10 | Cysteine- | atggcacgcgatattgatccttacaaggagttcggggcaaccgtcgagctgctgagcttcctg |
| | Modified | ccaagcgacttctttccatcagtgcgcgatctgctggataccgctagcgctctgtaccgggag |
| | HBc DNA | gctctcgagtccccagagcactctagccctcaccacacagcactgaggcaggccattctgtct |
| | sequence | tggggagagctcatgaccctcgcaacttggggggcgtgaacctggaggacccagcatctc |
| | | gcgacctcgtcgtgtcatacgtgaatacaaatatgggcctgaagtttagacagctgctgtggttt |
| | | cacatttcaagcctgacattcggacgcgagacagtgattgagtacctggtgtcatttggcgtgt |
| | | ggattagaacccctcccgcttacaggcctcctaatgccccaattctgtctactctgcccgagact |
| | | accgtcgtgcgcagacggggaagatctccacgccggcggactccatcacctagacggcgc |
| | | aggtcccagtctccaagacggcgccggtcacagtctagggagagccagtct |
| 11 | Truncated | MPLSYQHFRRLLLLDDEAGPLEEELPRLADEGLNRRVAED |
| | and | LNLGNLNVSIPWTHKVGNFTGLASSTVPVFNPHAKTPSFPN |
| | modified | IHLHQDIIKKCEQFVGPLTVNEKRRLQLIMPARFYPNVTKY |
| | Polymerase | LPLDKGIKPYYPEHLVNHYFQTRHALHTLWKAGILYKRET |
| | (Pol) | THSASFCGSPASWEQKLQDWGPCAEHGEHHIRIPRTPARVT |
| | Protein | GGVFLVDKNPHNTAESRLVVDFSQFSRGNYRVSWPKFAVP |
| | Sequence | NLQSLTNLLSSNLSWLSLDVSAAFYHLPLHPAAMPHLLVGS |
| | | SGLSRYVARLSSNSRIFNYQHGTMQNLHDSCSRNLYVSLLL |
| | | LYQTFGRKLHLYSHPIILGFRKIPMGVGLSPFLLAQFTSAGS |
| | | LPQDHIIQKIKECFRKLPVNRPIDWKVCQRIVGLLGFAAPFT |
| | | QCGYPALMPLYACIQSKQAFTFSPTYKAFLCKQYLNLYPV |
| | | ARQRPGLCQVFAHATPTGWGLVMGHQRMRGTFLASRKYT |
| | | SFPWLLGCAANWILRGTSFYYVPSALNPYHDPSRGRLGLSR |
| | | PLLRLPFRPTTGRTSLYADSPSVPSHLPDRVHFASPLHVA |
| 12 | Truncated | atgccactgtcttaccagcacttccggcggctgctgctcctcgatgatgaggcaggacccttg |
| | and | gaagaggaactaccacggctggccgacgagggactgaatcggcgagtcgccgaggatctc |
| | modified | aatctgggaaacctcaatgtctctatcccttggacacacaaggtcggaaattttactggcctcgc |
| | Polymerase | ttcttcaaccgtgcccgtgtttaacccacacgctaagacccctagctttcctaacattcacctgca |
| | (Pol) DNA | ccaggatattattaagaagtgcgagcagttcgtcgggccactgacagtgaacgagaagcgtc |
| | Sequence | gtttacagctgattatgcccgctaggttttaccctaatgtgaccaagtacctgcctctcgataagg |
| | | gaattaagccttactaccccgagcacctggtgaatcactactttcagacacgccacgcactcca |
| | | cacactgtggaaggctggcattctgtacaagagagagactacacactcagcatcattttgcgg |
| | | gtcacccgcttcttgggagcagaagctccaggactggggaccttgcgccgagcacggagag |
| | | caccacattcggattccccggactcccgctagggtgaccggggggtgttcctcgtcgataa |
| | | gaatccacacaatactgccgagtcacggctggtcgtcgattttagccagttttcacgcggaaat |
| | | taccgcgtctcttggccaaagtttgccgtgcctaacctccagtcactgaccaatctcctgtcatct |
| | | aatctgagctggctgagcctcgacgtcagcgcagccttctaccacctgccactccacccagc |
| | | agctatgccccacctgctcgtcggatcatcaggactctcacgttacgtcgctaggctgtcatca |
| | | aattctaggatctttaattaccagcacggaaccatgcagaatctccacgattcatgttcaagaaa |
| | | cctctatgtcagcctcctcctgctgtaccagacatttggaagaaagctccacctctactcccacc |
| | | ctattatcctcggctttagaaagatccctatgggagtcgggctgagccctttcctcctggcacag |
| | | tttactagcgcaggatcactgcctcaggatcacattattcagaagattaaagaatgttttagaaa |
| | | gctgcccgtgaataggccaattgattggaaggtgtgtcagagaatcgtcgggctcctcgggtt |
| | | cgccgccccttttacccagtgcggataccccgctctcatgcccctgtacgcatgtattcagtcta |
| | | agcaggcttttacttttagccctacatacaaggcatttctgtgtaagcagtacctgaatctctacc |
| | | ccgtcgcccggcagagacccggcctgtgtcaggtgtttgcacacgcaacccctacagggtg |
| | | gggactggtcatgggccaccagcgcatgagagggacatttctggcaagtaggaagtacacc |
| | | agctttccgtggctgctcgggtgcgccgctaattggatactgcgcggaacctcattttactatgt |
| | | gcctagcgcactgaacccataccacgacccttcacgggggcggctgggactgtctaggcct |
| | | ctgctccggctgccctttagacctacaactggaagaacttcactgtacgccgatagcccaagc |
| | | gtcccttcacacctccccgatagagtgcacttcgcctctccactgcacgtcgct |
| 13 | PD-L1 | acgttagctcgagaagaaggtatattgctgttgacagtgagcgacggacaaacagtgaccac |
| | shRNA | cttagtgaagcttcagatgtaaggtggtcactgtttgtccgctgcctactgcctcggacttcaag |
| | DNA | gggtcagtcagattttttctcgagaagaaggtatattgctgttgacagtgagcgacgctgaaagt |
| | Sequence | caatgccccatagtgaagcttcagatgtatggggcattgactttcagcgctgcctactgcctcg |
| | | gacttcaaggggtcagtcagaatttttctcgagaagaaggtatattgctgttgacagtgagcga |
| | | cgatttgctggcattatatatagtgaagcttcagatgtatatataatgccagcaaatcgctgccta |
| | | ctgcctcggacttcaaggggtcagtcagaatttttt |
| 14 | VSV G of | MLSYLIFALAVSPILGKIEIVFPQHTTGDWKRVPHEYNYCPT |
| | NJ serotype | SADKNSHGTQTGIPVELTMPKGLTTHQVEGFMCHSALWM |
| | Protein | TTCDFRWYGPKYITHSIHNEEPTDYQCLEAIKSYKDGVSFN |
| | Sequence | PGFPPQSCGYGTVTDAEAHIVTVTPHSVKVDEYTGEWIDPH |
| | | FIGGRCKGQICETVHNSTKWFTSSDGESVCSQLFTLVGGIFF |
| | | SDSEEITSMGLPETGIRSNYFPYISTEGICKMPFCRKQGYKL |
| | | KNDLWFQIMDPDLDKTVRDLPHIKDCDLSSSIITPGEHATDI |
| | | SLISDVERILDYALCQNTWSKIESGEPITPVDLSYLGPKNPG |
| | | VGPVFTIINGSLHYFTSKYLRVELESPVIPRMEGKVAGTRIV |
| | | RQLWDQWFPFGEVEIGPNGVLKTKQGYKFPLHIIGTGEVDS |
| | | DIKMERVVKHWEHPHIEAAQTFLKKDDTGEVLYYGDTGV |
| | | SKNPVELVEGWFSGWRSSLMGVLAVIIGFVILMFLIKLIGVL |
| | | SSLFRPKRRPIYKSDVEMAHFR |
| 15 | VSV G of | atgctgagctacctgatcttcgccctggccgtgtctcctatcctgggcaagatcgagatcgtgtt |
| | NJ Serotype | ccctcagcacaccaccggcgactggaaaagagtgccccacgagtacaactactgccccacc |
| | DNA | agcgccgacaagaatagccacggaacacagacaggcatccccgtggaactgaccatgcct |
| | Sequence | aagggcctgacaacccaccaggtggaaggcttcatgtgtcacagcgccctgtggatgacca |
| | | cctgtgactttcgttggtacggccccaagtacatcacccacagcatccacaacgaggaaccca |
| | | ccgactaccagtgcctggaagccatcaagagctacaaggacggcgtgtccttcaatcctgga |
| | | ttcccacctcagagctgcggctacggcacagtgacagatgccgaggctcacatcgtgaccgt |
| | | gacacctcacagcgtgaaggtggacgagtacacaggcgagtggatcgaccctcacttcatc |
| | | ggcggcagatgcaagggccagatctgcgagacagtgcacaacagcaccaagtggttcacc |
| | | agctccgatggcgagagcgtgtgcagccagctgtttaccctcgtcggcggcatcttcttcagc |
| | | gacagcgaagagatcaccagcatgggcctgcctgaaaccggaatcagaagcaactacttcc |
| | | cctacatcagcaccgagggaatctgcaagatgcccttctgtcggaagcagggctacaagctg |
| | | aagaacgacctgtggttccagatcatggaccccgacctggataagaccgtgcgggatctgcc |
| | | ccacatcaaggactgtgatctgagcagcagcatcatcacccctggcgagcacgccacagac |
| | | atcagcctgatcagcgacgtggaacggatcctggactacgccctgtgccagaacacctggtc |
| | | taagatcgagtccggcgagcccatcacacccgtggatctgtcttatctgggccccaagaatcc |
| | | tggcgtgggccctgtgttcaccatcatcaatggcagcctgcactacttcaccagcaagtacctg |
| | | agagtggaactggaaagccctgtgatccccagaatggaaggcaaggtggccggcacaaga |
| | | atcgtcagacagctgtgggaccagtggttcccattcggcgaggtggaaatcggccctaacgg |
| | | cgtgctgaaaacaaagcaggggtataagttcccgctgcacatcatcggcaccggcgaagtg |
| | | gacagcgacatcaagatggaacgggtcgtgaagcactgggagcaccctcacattgaggcc |
| | | gctcagaccttcctgaagaaggacgatacaggcgaggtgctgtactacggcgataccgggg |
| | | tgtcaaagaaccccgtcgaactggtggaaggatggtttagcggatggcggtctagcctgatg |
| | | ggagtgctggccgtgatcatcggcttcgtgatcctgatgttcctgattaagctgatcggggtgct |
| | | gagcagcctgttcaga |
| 16 | CARG- | ctagtgcatccaaagaattcaaaaagcttctcgagagtacttctagagcggccgcgcatcgatt |
| | 201(IN) | ttccacccgggtggggtaccaggtaagtgtacccaattcgccctatagtgagtcgtattacaatt |
| | Plasmid A | cactggccgtcgttttacaacgtcgtgactgggaaaaccctggcgttacccaacttaatcgcctt |
| | | gcagcacatccccctttcgccagctggcgtaatagcgaagaggcccgcaccgatcgcccttc |
| | | ccaacagttgcgcagcctgaatggcgaatggagatccaatttttaagtgtataatgtgttaaact |
| | | actgattctaattgtttgtgtattttagattcacagtcccaaggctcatttcaggcccctcagtcctc |
| | | acagtctgttcatgatcataatcagccataccacatttgtagaggttttacttgctttaaaaaacct |
| | | cccacacctccccctgaacctgaaacataaaatgaatgcaattgttgttgttaacttgtttattgca |
| | | gcttataatggttacaaataaagcaatagcatcacaaatttcacaaataaagcatttttttcactgc |
| | | attctagttgtggtttgtccaaactcatcaatgtatcttaacgcgtaaattgtaagcgttaatattttg |
| | | ttaaaattcgcgttaaatttttgttaaatcagctcattttttaaccaataggccgaaatcggcaaaat |
| | | cccttataaatcaaaagaatagaccgagatagggttgagtgttgttccagtttggaacaagagt |
| | | ccactattaaagaacgtggactccaacgtcaaagggcgaaaaaccgtctatcagggcgatgg |
| | | cccactacgtgaaccatcaccctaatcaagttttttggggtcgaggtgccgtaaagcactaaat |
| | | cggaaccctaaagggagcccccgatttagagcttgacggggaaagccggcgaacgtggcg |
| | | agaaaggaagggaagaaagcgaaaggagcgggcgctagggcgctggcaagtgtagcgg |
| | | tcacgctgcgcgtaaccaccacacccgccgcgcttaatgcgccgctacagggcgcgtcagg |
| | | tggcacttttcggggaaatgtgcgcggaacccctatttgtttatttttctaaatacattcaaatatgt |
| | | atccgctcatgagacaataaccctgataaatgcttcaataatattgaaaaaggaagagtcctga |
| | | ggcggaaagaaccagctgtggaatgtgtgtcagttagggtgtggaaagtccccaggctcccc |
| | | agcaggcagaagtatgcaaagcatgcatctcaattagtcagcaaccaggtgtggaaagtccc |
| | | caggctccccagcaggcagaagtatgcaaagcatgcatctcaattagtcagcaaccatagtc |
| | | ccgcccctaactccgcccatcccgcccctaactccgcccagttccgcccattctccgccccat |
| | | ggctgactaattttttttatttatgcagaggccgaggccgcctcggcctctgagctattccagaa |
| | | gtagtgaggaggcttttttggaggcctaggcttttgcaaagatcgatcaagagacaggatgag |
| | | gatcgtttcgcatgattgaacaagatggattgcacgcaggttctccggccgcttgggtggaga |
| | | ggctattcggctatgactgggcacaacagacaatcggctgctctgatgccgccgtgttccggc |
| | | tgtcagcgcaggggcgcccggttctttttgtcaagaccgacctgtccggtgccctgaatgaact |
| | | gcaagacgaggcagcgcggctatcgtggctggccacgacgggcgttccttgcgcagctgtg |
| | | ctcgacgttgtcactgaagcgggaagggactggctgctattgggcgaagtgccggggcagg |
| | | atctcctgtcatctcaccttgctcctgccgagaaagtatccatcatggctgatgcaatgcggcg |
| | | gctgcatacgcttgatccggctacctgcccattcgaccaccaagcgaaacatcgcatcgagc |
| | | gagcacgtactcggatggaagccggtcttgtcgatcaggatgatctggacgaagagcatcag |
| | | gggctcgcgccagccgaactgttcgccaggctcaaggcgagcatgcccgacggcgaggat |
| | | ctcgtcgtgacccatggcgatgcctgcttgccgaatatcatggtggaaaatggccgcttttctg |
| | | gattcatcgactgtggccggctgggtgtggcggaccgctatcaggacatagcgttggctacc |
| | | cgtgatattgctgaagagcttggcggcgaatgggctgaccgcttcctcgtgctttacggtatcg |
| | | ccgctcccgattcgcagcgcatcgccttctatcgccttcttgacgagttcttctgagcgggactc |
| | | tggggttcgaaatgaccgaccaagcgacgcccaacctgccatcacgagatttcgattccacc |
| | | gccgccttctatgaaaggttgggcttcggaatcgttttccgggacgccggctggatgatcctcc |
| | | agcgcggggatctcatgctggagttcttcgcccaccctagggggaggctaactgaaacacg |
| | | gaaggagacaataccggaaggaacccgcgctatgacggcaataaaaagacagaataaaac |
| | | gcacggtgttgggtcgtttgttcataaacgcggggttcggtcccagggctggcactctgtcgat |
| | | accccaccgagaccccattggggccaatacgcccgcgtttcttccttttccccaccccacccc |
| | | ccaagttcgggtgaaggcccagggctcgcagccaacgtcggggcggcaggccctgccata |
| | | gcctcaggttactcatatatactttagattgatttaaaacttcatttttaatttaaaaggatctaggtg |
| | | aagatcctttttgataatctcatgaccaaaatcccttaacgtgagttttcgttccactgagcgtcag |
| | | accccgtagaaaagatcaaaggatcttcttgagatcctttttttctgcgcgtaatctgctgcttgca |
| | | aacaaaaaaaccaccgctaccagcggtggtttgtttgccggatcaagagctaccaactcttttt |
| | | ccgaaggtaactggcttcagcagagcgcagataccaaatactgttcttctagtgtagccgtagt |
| | | taggccaccacttcaagaactctgtagcaccgcctacatacctcgctctgctaatcctgttacca |
| | | gtggctgctgccagtggcgataagtcgtgtcttaccgggttggactcaagacgatagttaccg |
| | | gataaggcgcagcggtcgggctgaacggggggttcgtgcacacagcccagcttggagcga |
| | | acgacctacaccgaactgagatacctacagcgtgagctatgagaaagcgccacgcttcccga |
| | | agggagaaaggcggacaggtatccggtaagcggcagggtcggaacaggagagcgcacg |
| | | agggagcttccagggggaaacgcctggtatctttatagtcctgtcgggtttcgccacctctgac |
| | | ttgagcgtcgatttttgtgatgctcgtcaggggggcggagcctatggaaaaacgccagcaac |
| | | gcggcctttttacggttcctggccttttgctggccttttgctcacatgttctttcctgcgttatcccct |
| | | gattctgtggataaccgtattaccgccatgcattagttattaatagtaatcaattacggggtcatta |
| | | gttcatagcccatatatggagttccgcgttacataacttacggtaaatggcccgcctggctgac |
| | | cgcccaacgacccccgcccattgacgtcaataatgacgtatgttcccatagtaacgccaatag |
| | | ggactttccattgacgtcaatgggtggagtatttacggtaaactgcccacttggcagtacatcaa |
| | | gtgtatcatatgccaagtacgccccctattgacgtcaatgacggtaaatggcccgcctggcatt |
| | | atgcccagtacatgaccttatgggactttcctacttggcagtacatctacgtattagtcatcgctat |
| | | taccatggtgatgcggttttggcagtacatcaatgggcgtggatagcggtttgactcacgggg |
| | | atttccaagtctccaccccattgacgtcaatgggagtttgttttggcaccaaaatcaacgggact |
| | | ttccaaaatgtcgtaacaactccgccccattgacgcaaatgggcggtaggcgtgtacggtgg |
| | | gaggtctatataagcagagctggtttagtgaaccgtatggcggatgtgtgacatacacgacgc |
| | | caaaagattttgttccagctcctgccacctccgctacgcgagagattaaccacccacgatggc |
| | | cgccaaagtgcatgttgatattgaggctgacagcccattcatcaagtctttgcagaaggcatttc |
| | | cgtcgttcgaggtggagtcattgcaggtcacaccaaatgaccatgcaaatgccagagcattttc |
| | | gcacctggctaccaaattgatcgagcaggagactgacaaagacacactcatcttggatatcgg |
| | | cagtgcgccttccaggagaatgatgtctacgcacaaataccactgcgtatgccctatgcgcag |
| | | cgcagaagaccccgaaaggctcgtatgctacgcaaagaaactggcagcggcctccgagaa |
| | | ggtgctggatagagagatcgcaggaaaaatcaccgacctgcagaccgtcatggctacgcca |
| | | gacgctgaatctcctaccttttgcctgcatacagacgtcacgtgtcgtacggcagccgaagtg |
| | | gccgtataccaggacgtgtatgctgtacatgcaccaacatcgctgtaccatcaggcgatgaaa |
| | | ggtgtcagaacggcgtattggattgggtttgacaccaccccgtttatgtttgacgcgctagcag |
| | | gcgcgtatccaacctacgccacaaactgggccgacgagcaggtgttacaggccaggaacat |
| | | aggactgtgtgcagcatccttgactgagggaagactcggcaaactgtccattctccgcaagaa |
| | | gcaattgaaaccttgcgacacagtcatgttctcggtaggatctacattgtacactgagagcaga |
| | | aagctactgaggagctggcacttaccctccgtattccacctgaaaggtaaacaatcctttacct |
| | | gtaggtgcgataccatcgtatcatgtgaagggtacgtagttaagaaaatcactatgtgccccgg |
| | | cctgtacggtaaaacggtagggtacgccgtgacgtatcacgcggagggattcctagtgtgca |
| | | agaccacagacactgtcaaaggagaaagagtctcattccctgtatgcacctacgtcccctcaa |
| | | ccatctgtgatcaaatgactggcatactggcgaccgacatcacaccggaggacgcacagaa |
| | | gttgttagtgggattgaatcagaggatagttgtgaacggaagaacacagcgaaacactaacac |
| | | gatgaagaactatctgcttccgattgtggccgtcgcatttagcaagtgggcgagggaatacaa |
| | | ggcagaccttgatgatgaaaaacctctgggtgtccgagagaggtcacttacttgctgctgcttg |
| | | tgggcatttaaaacgaggaagatgcacaccatgtacaagaaaccagacacccagacaatagt |
| | | gaaggtgccttcagagtttaactcgttcgtcatcccgagcctatggtctacaggcctcgcaatc |
| | | ccagtcagatcacgcattaagatgcttttggccaagaagaccaagcgagagtcaatacctgtt |
| | | ctcgacgcgtcgtcagccagggatgctgaacaagaggagaaggagaggttggaggccga |
| | | gctgactagagaagccttaccacccctcgtccccaccgcgccggcggagacgggagtcgtc |
| | | gacgtcgacgttgaagaactagagtatcacgcaggtgcaggggtcgtggaaacacctcgca |
| | | gcgcgttgaaagtcaccgcacagccgaacggcgtactactaggaaattacgtagttctgtccc |
| | | cgcagaccgtgctcaagagctccaagttggcccccgtgcaccctctagcagagcaggtgaa |
| | | aataataacacataacgggagggccggccgttaccaggtcgacggatatgacggcagggtc |
| | | ctactaccatgtggatcggccattccggtccctgagtttcaagctttgagcgagagcgccacta |
| | | tggtgtacaacgaaagggagttcgtcaacaggaaactataccatattgccgttcacggaccgt |
| | | cgctgaacaccgacgaggagaactacgagaaagtcagagctgaaagaactgacgccgagt |
| | | acgtgttcgacgtagataaaaaatgctgcgtcaagagagaggaagcgtcgggtttggtgttgg |
| | | tgggagagctaaccaaccccccgttccatgaattcgcctacgaagggctgaagatcaggccg |
| | | tcggcaccatataagactacagtagtaggagtctttggggttccgggatcaggcaagtctgcta |
| | | ttattaagagcctcgtgaccaaacacgatctggtcaccagcggcaagaaggagaactgccag |
| | | gaaatagtcaacgacgtgaagaagcaccgcggactggacatccaggcaaaaacagtggac |
| | | tccatcctgctaaacgggtgtcgtcgtgccgtggacatcctatatgtggacgaggctttcgcttg |
| | | ccattccggtactctgctagccctaattgctcttgttaaacctcggagcaaagtggtgttatgcg |
| | | gagaccccaagcaatgcggattcttcaatatgatgcagcttaaggtgaacttcaaccacaacat |
| | | ctgcactgaagtatgtcataaaagtatatccagacgttgcacgcgtccagtcacggccatcgtg |
| | | tctacattgcactacggaggcaagatgcgcacgaccaacccgtgcaacaaacccataatcat |
| | | agacaccacaggacagaccaagcccaagccaggagacatcgtgttaacatgcttccgaggc |
| | | tgggtaaagcagctgcagttggactaccgtggacacgaagtcatgacagcagcagcatctca |
| | | gggcctcacccgcaaaggggtatacgccgtaaggcagaaggtgaatgaaaatcccttgtatg |
| | | cccctgcgtcggagcacgtgaatgtactgctgacgcgcactgaggataggctggtgtggaaa |
| | | acgctggccggcgatccctggattaaggtcctatcaaacattccacagggtaactttacggcc |
| | | acattggaagaatggcaagaagaacacgacaaaataatgaaggtgattgaaggaccggctg |
| | | cgcctgtggacgcgttccagaacaaagcgaacgtgtgttgggcgaaaagcctggtgcctgtc |
| | | ctggacactgccggaatcagattgacagcagaggagtggagcaccataattacagcatttaa |
| | | ggaggacagagcttactctccagtggtggccttgaatgaaatttgcaccaagtactatggagtt |
| | | gacctggacagtggcctgttttctgccccgaaggtgtccctgtattacgagaacaaccactgg |
| | | gataacagacctggtggaaggatgtatggattcaatgccgcaacagctgccaggctggaagc |
| | | tagacataccttcctgaaggggcagtggcatacgggcaagcaggcagttatcgcagaaaga |
| | | aaaatccaaccgctttctgtgctggacaatgtaattcctatcaaccgcaggctgccgcacgccc |
| | | tggtgactgagtacaagacggttaaaggcagtagggttgagtggctggtcaataaagtaaga |
| | | gggtaccacgtcctgctggtgagtgagtacaacctggctttgcctcgacgcagggtcacttgg |
| | | ttgtcaccgctgaatgtcacaggcgccgataggtgctacgacctaagtttaggactgccggct |
| | | gacgccggcaggttcgacttggtctttgtgaacattcacacggaattcagaatccaccactacc |
| | | agcagtgtgtcgaccacgccatgaagctgcagatgcttgggggagatgcgctacgactgcta |
| | | aaacccggcggcagcctcttgatgagagcttacggatacgccgataaaatcagcgaagccgt |
| | | tgtttcctccttaagcagaaagttctcgtctgcaagagtgttgcgcccggattgtgtcaccagca |
| | | atacagaagtgttcttgctgttctccaactttgacaacggaaagagaccctctacgctacacca |
| | | gatgaataccaagctgagtgccgtgtatgccggagaagccatgcacacggccgggtgtgca |
| | | ccatcctacagagttaagagagcagacatagccacgtgcacagaagcggctgtggttaacgc |
| | | agctaacgcccgtggaactgtaggggatggcgtatgcagggccgtggcgaagaaatggcc |
| | | gtcagcctttaagggagaagcaacaccagtgggcacaattaaaacagtcatgtgcggctcgt |
| | | accccgtcatccacgctgtagcgcctaatttctctgccacgactgaagcggaagggaaccgc |
| | | gaattggccgctgtctaccgggcagtggccgccgaagtaaacagactgtcactgagcagcgt |
| | | agccatcccgctgctgtccacaggagtgttcagcggcggaagagataggctgcagcaatcc |
| | | ctcaaccatctattcacagcaatggacgccacggacgctgacgtgaccatctactgcagaga |
| | | caaaagttgggagaagaaaatccaggaagccatagacacgaggacggctgtggagttgctc |
| | | aatgatgacgtggagctgaccacagacttggtgagagtgcacccggacagcagcctggtgg |
| | | gtcgtaagggctacagtaccactgacgggtcgctgtactcgtactttgaaggtacgaaattcaa |
| | | ccaggctgctattgatatggcagagatactgacgttgtggcccagactgcaagaggcaaacg |
| | | aacagatatgcctatacgcgctgggcgaaacaatggacaacatcagatccaaatgtccggtg |
| | | aacgattccgattcatcaacacctcccaggacagtgccctgcctgtgccgctacgcaatgaca |
| | | gcagaacggatcacccgccttaggtcacaccaagttaaaagcatggtggtttgctcatcttttcc |
| | | cctcccgaaataccatgtagatggggtgcagaaggtaaagtgcgagaaggttctcctgttcga |
| | | cccgacggtaccttcagtggttagtccgcggaagtatgccgcatctacgacggaccactcag |
| | | atcggtcgttacgagggtttgacttggactggaccaccgactcgtcttccactgccagcgatac |
| | | catgtcgctacccagtttgcagtcgtgtgacatcgactcgatctacgagccaatggctcccata |
| | | gtagtgacggctgacgtacaccctgaacccgcaggcatcgcggacctggcggcagatgtgc |
| | | atcctgaacccgcagaccatgtggacctcgagaacccgattcctccaccgcgcccgaagag |
| | | agctgcataccttgcctcccgcgcggcggagcgaccggtgccggcgccgagaaagccgac |
| | | gcctgccccaaggactgcgtttaggaacaagctgcctttgacgttcggcgactttgacgagca |
| | | cgaggtcgatgcgttggcctccgggattactttcggagacttcgacgacgtcctgcgactagg |
| | | ccgcgcgggtgcatatattttctcctcggacactggcagcggacatttacaacaaaaatccgtt |
| | | aggcagcacaatctccagtgcgcacaactggatgcggtcgaggaggagaaaatgtacccgc |
| | | caaaattggatactgagagggagaagctgttgctgctgaaaatgcagatgcacccatcggag |
| | | gctaataagagtcgataccagtctcgcaaagtggagaacatgaaagccacggtggtggaca |
| | | ggctcacatcgggggccagattgtacacgggagcggacgtaggccgcataccaacatacg |
| | | cggttcggtacccccgccccgtgtactcccctaccgtgatcgaaagattctcaagccccgatg |
| | | tagcaatcgcagcgtgcaacgaatacctatccagaaattacccaacagtggcgtcgtaccag |
| | | ataacagatgaatacgacgcatacttggacatggttgacgggtcggatagttgcttggacaga |
| | | gcgacattctgcccggcgaagctccggtgctacccgaaacatcatgcgtaccaccagccga |
| | | ctgtacgcagtgccgtcccgtcaccctttcagaacacactacagagcgtgctagcggccgcc |
| | | accaagagaaactgcaacgtcacgcaaatgcgagaactacccaccatggactcggcagtgtt |
| | | caacgtggagtgcttcaagcgctatgcctgctccggagaatattgggaagaatatgctaaaca |
| | | acctatccggataaccactgagaacatcactacctatgtgaccaaattgaaaggcccgaaagc |
| | | tgctgccttgttcgctaagacccacaacttggttccgctgcaggaggttcccatggacagattc |
| | | acggtcgacatgaaacgagatgtcaaagtcactccagggacgaaacacacagaggaaaga |
| | | cccaaagtccaggtaattcaagcagcggagccattggcgaccgcttacctgtgcggcatcca |
| | | cagggaattagtaaggagactaaatgctgtgttacgccctaacgtgcacacattgtttgatatgt |
| | | cggccgaagactttgacgcgatcatcgcctctcacttccacccaggagacccggttctagaga |
| | | cggacattgcatcattcgacaaaagccaggacgactccttggctcttacaggtttaatgatcctc |
| | | gaagatctaggggtggatcagtacctgctggacttgatcgaggcagcctttggggaaatatcc |
| | | agctgtcacctaccaactggcacgcgcttcaagttcggagctatgatgaaatcgggcatgtttc |
| | | tgactttgtttattaacactgttttgaacatcaccatagcaagcagggtactggagcagagactc |
| | | actgactccgcctgtgcggccttcatcggcgacgacaacatcgttcacggagtgatctccgac |
| | | aagctgatggcggagaggtgcgcgtcgtgggtcaacatggaggtgaagatcattgacgctgt |
| | | catgggcgaaaaacccccatatttttgtgggggattcatagtttttgacagcgtcacacagacc |
| | | gcctgccgtgtttcagacccacttaagcgcctgttcaagttgggtaagccgctaacagctgaa |
| | | gacaagcaggacgaagacaggcgacgagcactgagtgacgaggttagcaagtggttccgg |
| | | acaggcttgggggccgaactggaggtggcactaacatctaggtataaggtagagggctgca |
| | | aaagtatcctcatagccatggccaccttggcgagggacattaaggcgtttaagaaattgagag |
| | | gacctgttatacacctctacggcggtcctagattggtgcgttaatacacagaattctgattggatc |
| | | cacgatggcacgcgatattgatccttacaaggagttcggggcaaccgtcgagctgctgagctt |
| | | cctgccaagcgacttctttccatcagtgcgcgatctgctggataccgctagcgctctgtaccgg |
| | | gaggctctcgagtccccagagcactctagccctcaccacacagcactgaggcaggccattct |
| | | gtcttggggagagctcatgaccctcgcaacttggggggcgtgaacctggaggacccagcat |
| | | ctcgcgacctcgtcgtgtcatacgtgaatacaaatatgggcctgaagtttagacagctgctgtg |
| | | gtttcacatttcaagcctgacattcggacgcgagacagtgattgagtacctggtgtcatttggcg |
| | | tgtggattagaacccctcccgcttacaggcctcctaatgccccaattctgtctactctgcccgag |
| | | actaccgtcgtgcgcagacggggaagatctccacgccggcggactccatcacctagacggc |
| | | gcaggtcccagtctccaagacggcgccggtcacagtctagggagagccagtctgagggaa |
| | | gaggcagcctgctgacctgcggcgacgtggaggagaacccaggcccaatgaagtgcctttt |
| | | gtacttagcctttttattcattggggtgaattgcaagttcaccatagtttttccacacaaccaaaaa |
| | | ggaaactggaaaaatgttccttctaattaccattattgcccgtcaagctcagatttaaattggcat |
| | | gatgacttaataggcacagccttacaagtcaaaatgcccaagagtcacaaggctattcaagca |
| | | gacggttggatgtgtcatgcttccaaatgggtcactacttgtgacttccgctggtatggaccgaa |
| | | gtatataacacattccatccgatccttcactccatctgtagaacaatgcaaggaaagcattgaac |
| | | aaacgaaacaaggaacttggctgaatccaggcttccctcctcaaagttgtggatatgcaactgt |
| | | gacggatgccgaagcagtgattgtccaggtgactcctcaccatgtgctggttgatgaatacac |
| | | aggagaatgggttgattcacagttcatcaacggaaaatgcagcaattacatatgccccactgtc |
| | | cataactctacaacctggcattctgactataaggtcaaagggctatgtgattctaacctcatttcc |
| | | atggacatcaccttcttctcagaggacggagagctatcatccctgggaaaggagggcacagg |
| | | gttcagaagtaactactttgcttatgaaactggaggcaaggcctgcaaaatgcaatactgcaag |
| | | cattggggagtcagactcccatcaggtgtctggttcgagatggctgataaggatctctttgctgc |
| | | agccagattccctgaatgcccagaagggtcaagtatctctgctccatctcagacctcagtggat |
| | | gtaagtctaattcaggacgttgagaggatcttggattattccctctgccaagaaacctggagca |
| | | aaatcagagcgggtcttccaatctctccagtggatctcagctatcttgctcctaaaaacccagg |
| | | aaccggtcctgctttcaccataatcaatggtaccctaaaatactttgagaccagatacatcagag |
| | | tcgatattgctgctccaatcctctcaagaatggtcggaatgatcagtggaactaccacagaaag |
| | | ggaactgtgggatgactgggcaccatatgaagacgtggaaattggacccaatggagttctga |
| | | ggaccagttcaggatataagtttcctttatacatgattggacatggtatgttggactccgatcttca |
| | | tcttagctcaaaggctcaggtgttcgaacatcctcacattcaagacgctgcttcgcaacttcctg |
| | | atgatgagagtttattttttggtgatactgggctatccaaaaatccaatcgagcttgtagaaggtt |
| | | ggttcagtagttggaaaagctctattgcctcttttttctttatcatagggttaatcattggactattctt |
| | | ggttctccgagttggtatccatctttgcattaaattaaagcacaccaagaaaagacagatttatac |
| | | agacatagagatgaaccgacttggaaagtaattaattaataatcatattaagggcccaagggg |
| | | ctgcaaaagtatcctcatagccatggccaccttggcgagggacattaaggcgtttaagaaattg |
| | | agaggacctgttatacacctctacggcggtcctagattggtgcgttaatacacagaattctgatt |
| | | ggcgcgccacgatgcagtggaattccacaaccttccaccaaactctgcaagatcccagagtg |
| | | agaggcctgtatttccctgctggtggctccagttcaggaacagtaaaccctgttctgactactgc |
| | | ctctcccttatcgtcaatcttctcgaggattggggaccctgcgctgaacatggagaacatcacat |
| | | caggattcctaggaccccttctcgtgttacaggcggggtttttcttgttgacaagaatcctcacaa |
| | | taccgcagagtctagactcgtggtggacttctctcaattttctagggggaactaccgtgtgtcttg |
| | | gccaaaattcgcagtccccaacctccaatcactcaccaacctcttgtcctccaacttgtcctggt |
| | | tatcgctggatgtgtctgcggcgttttatcatcttcctcttcatcctgctgctatgcctcatcttcttgt |
| | | tggttcttctggactatcaaggtatgttgcccgtttgtcctctaattccaggatcctcaacaaccag |
| | | cacgggaccatgccggacctgcatgactactgctcaaggaacctctatgtatccctcctgttgc |
| | | tgtaccaaaccttcggacggaaattgcacctgtattcccatcccatcatcctgggctttcggaaa |
| | | attcctatgggagtgggcctcagcccgtttctcctggctcagtttactagtgccatttgttcagtg |
| | | gttcgtagggctttcccccactgtttggctttcagttatatggatgatgtggtattgggggccaag |
| | | tctgtacagcatcttgagtccctttttaccgctgttaccaattttcttttgtctttgggtatacatttaac |
| | | ctgcaggaccataactgtataacttgtaacaaagcgcaacaagacctgcgcaattggccccgt |
| | | ggtccgcctcacggaaactcggggcaactcatattgacacattaattggcaataattggaagct |
| | | tacataagcttaattcgacgaataattggatttttattttattttgcaattggtttttaatatttccaaaa |
| | | aaaaaaaaaaaaaaaaaaaaaaaaaaaaaaaaaaaaaaaaaaaaaaaaaaaaaaaaaa |
| 17 | CARG- | ctagtgcatccaaagaattcaaaaagcttctcgagagtacttctagagcggccgcgcatcgatt |
| | 201(IN) | ttccacccgggtggggtaccaggtaagtgtacccaattcgccctatagtgagtcgtattacaatt |
| | Plasmid B | cactggccgtcgttttacaacgtcgtgactgggaaaaccctggcgttacccaacttaatcgcctt |
| | | gcagcacatccccctttcgccagctggcgtaatagcgaagaggcccgcaccgatcgcccttc |
| | | ccaacagttgcgcagcctgaatggcgaatggagatccaatttttaagtgtataatgtgttaaact |
| | | actgattctaattgtttgtgtattttagattcacagtcccaaggctcatttcaggcccctcagtcctc |
| | | acagtctgttcatgatcataatcagccataccacatttgtagaggttttacttgctttaaaaaacct |
| | | cccacacctccccctgaacctgaaacataaaatgaatgcaattgttgttgttaacttgtttattgca |
| | | gcttataatggttacaaataaagcaatagcatcacaaatttcacaaataaagcatttttttcactgc |
| | | attctagttgtggtttgtccaaactcatcaatgtatcttaacgcgtaaattgtaagcgttaatattttg |
| | | ttaaaattcgcgttaaatttttgttaaatcagctcattttttaaccaataggccgaaatcggcaaaat |
| | | cccttataaatcaaaagaatagaccgagatagggttgagtgttgttccagtttggaacaagagt |
| | | ccactattaaagaacgtggactccaacgtcaaagggcgaaaaaccgtctatcagggcgatgg |
| | | cccactacgtgaaccatcaccctaatcaagttttttggggtcgaggtgccgtaaagcactaaat |
| | | cggaaccctaaagggagcccccgatttagagcttgacggggaaagccggcgaacgtggcg |
| | | agaaaggaagggaagaaagcgaaaggagcgggcgctagggcgctggcaagtgtagcgg |
| | | tcacgctgcgcgtaaccaccacacccgccgcgcttaatgcgccgctacagggcgcgtcagg |
| | | tggcacttttcggggaaatgtgcgcggaacccctatttgtttatttttctaaatacattcaaatatgt |
| | | atccgctcatgagacaataaccctgataaatgcttcaataatattgaaaaaggaagagtcctga |
| | | ggcggaaagaaccagctgtggaatgtgtgtcagttagggtgtggaaagtccccaggctcccc |
| | | agcaggcagaagtatgcaaagcatgcatctcaattagtcagcaaccaggtgtggaaagtccc |
| | | caggctccccagcaggcagaagtatgcaaagcatgcatctcaattagtcagcaaccatagtc |
| | | ccgcccctaactccgcccatcccgcccctaactccgcccagttccgcccattctccgccccat |
| | | ggctgactaattttttttatttatgcagaggccgaggccgcctcggcctctgagctattccagaa |
| | | gtagtgaggaggcttttttggaggcctaggcttttgcaaagatcgatcaagagacaggatgag |
| | | gatcgtttcgcatgattgaacaagatggattgcacgcaggttctccggccgcttgggtggaga |
| | | ggctattcggctatgactgggcacaacagacaatcggctgctctgatgccgccgtgttccggc |
| | | tgtcagcgcaggggcgcccggttctttttgtcaagaccgacctgtccggtgccctgaatgaact |
| | | gcaagacgaggcagcgcggctatcgtggctggccacgacgggcgttccttgcgcagctgtg |
| | | ctcgacgttgtcactgaagcgggaagggactggctgctattgggcgaagtgccggggcagg |
| | | atctcctgtcatctcaccttgctcctgccgagaaagtatccatcatggctgatgcaatgcggcg |
| | | gctgcatacgcttgatccggctacctgcccattcgaccaccaagcgaaacatcgcatcgagc |
| | | gagcacgtactcggatggaagccggtcttgtcgatcaggatgatctggacgaagagcatcag |
| | | gggctcgcgccagccgaactgttcgccaggctcaaggcgagcatgcccgacggcgaggat |
| | | ctcgtcgtgacccatggcgatgcctgcttgccgaatatcatggtggaaaatggccgcttttctg |
| | | gattcatcgactgtggccggctgggtgtggcggaccgctatcaggacatagcgttggctacc |
| | | cgtgatattgctgaagagcttggcggcgaatgggctgaccgcttcctcgtgctttacggtatcg |
| | | ccgctcccgattcgcagcgcatcgccttctatcgccttcttgacgagttcttctgagcgggactc |
| | | tggggttcgaaatgaccgaccaagcgacgcccaacctgccatcacgagatttcgattccacc |
| | | gccgccttctatgaaaggttgggcttcggaatcgttttccgggacgccggctggatgatcctcc |
| | | agcgcggggatctcatgctggagttcttcgcccaccctagggggaggctaactgaaacacg |
| | | gaaggagacaataccggaaggaacccgcgctatgacggcaataaaaagacagaataaaac |
| | | gcacggtgttgggtcgtttgttcataaacgcggggttcggtcccagggctggcactctgtcgat |
| | | accccaccgagaccccattggggccaatacgcccgcgtttcttccttttccccaccccacccc |
| | | ccaagttcgggtgaaggcccagggctcgcagccaacgtcggggcggcaggccctgccata |
| | | gcctcaggttactcatatatactttagattgatttaaaacttcatttttaatttaaaaggatctaggtg |
| | | aagatcctttttgataatctcatgaccaaaatcccttaacgtgagttttcgttccactgagcgtcag |
| | | accccgtagaaaagatcaaaggatcttcttgagatcctttttttctgcgcgtaatctgctgcttgca |
| | | aacaaaaaaaccaccgctaccagcggtggtttgtttgccggatcaagagctaccaactcttttt |
| | | ccgaaggtaactggcttcagcagagcgcagataccaaatactgttcttctagtgtagccgtagt |
| | | taggccaccacttcaagaactctgtagcaccgcctacatacctcgctctgctaatcctgttacca |
| | | gtggctgctgccagtggcgataagtcgtgtcttaccgggttggactcaagacgatagttaccg |
| | | gataaggcgcagcggtcgggctgaacggggggttcgtgcacacagcccagcttggagcga |
| | | acgacctacaccgaactgagatacctacagcgtgagctatgagaaagcgccacgcttcccga |
| | | agggagaaaggcggacaggtatccggtaagcggcagggtcggaacaggagagcgcacg |
| | | agggagcttccagggggaaacgcctggtatctttatagtcctgtcgggtttcgccacctctgac |
| | | ttgagcgtcgatttttgtgatgctcgtcaggggggcggagcctatggaaaaacgccagcaac |
| | | gcggcctttttacggttcctggccttttgctggccttttgctcacatgttctttcctgcgttatcccct |
| | | gattctgtggataaccgtattaccgccatgcattagttattaatagtaatcaattacggggtcatta |
| | | gttcatagcccatatatggagttccgcgttacataacttacggtaaatggcccgcctggctgac |
| | | cgcccaacgacccccgcccattgacgtcaataatgacgtatgttcccatagtaacgccaatag |
| | | ggactttccattgacgtcaatgggtggagtatttacggtaaactgcccacttggcagtacatcaa |
| | | gtgtatcatatgccaagtacgccccctattgacgtcaatgacggtaaatggcccgcctggcatt |
| | | atgcccagtacatgaccttatgggactttcctacttggcagtacatctacgtattagtcatcgctat |
| | | taccatggtgatgcggttttggcagtacatcaatgggcgtggatagcggtttgactcacgggg |
| | | atttccaagtctccaccccattgacgtcaatgggagtttgttttggcaccaaaatcaacgggact |
| | | ttccaaaatgtcgtaacaactccgccccattgacgcaaatgggggtaggcgtgtacggtgg |
| | | gaggtctatataagcagagctggtttagtgaaccgtatggcggatgtgtgacatacacgacgc |
| | | caaaagattttgttccagctcctgccacctccgctacgcgagagattaaccacccacgatggc |
| | | cgccaaagtgcatgttgatattgaggctgacagcccattcatcaagtctttgcagaaggcatttc |
| | | cgtcgttcgaggtggagtcattgcaggtcacaccaaatgaccatgcaaatgccagagcattttc |
| | | gcacctggctaccaaattgatcgagcaggagactgacaaagacacactcatcttggatatcgg |
| | | cagtgcgccttccaggagaatgatgtctacgcacaaataccactgcgtatgccctatgcgcag |
| | | cgcagaagaccccgaaaggctcgtatgctacgcaaagaaactggcagcggcctccgagaa |
| | | ggtgctggatagagagatcgcaggaaaaatcaccgacctgcagaccgtcatggctacgcca |
| | | gacgctgaatctcctaccttttgcctgcatacagacgtcacgtgtcgtacggcagccgaagtg |
| | | gccgtataccaggacgtgtatgctgtacatgcaccaacatcgctgtaccatcaggcgatgaaa |
| | | ggtgtcagaacggcgtattggattgggtttgacaccaccccgtttatgtttgacgcgctagcag |
| | | gcgcgtatccaacctacgccacaaactgggccgacgagcaggtgttacaggccaggaacat |
| | | aggactgtgtgcagcatccttgactgagggaagactcggcaaactgtccattctccgcaagaa |
| | | gcaattgaaaccttgcgacacagtcatgttctcggtaggatctacattgtacactgagagcaga |
| | | aagctactgaggagctggcacttaccctccgtattccacctgaaaggtaaacaatcctttacct |
| | | gtaggtgcgataccatcgtatcatgtgaagggtacgtagttaagaaaatcactatgtgccccgg |
| | | cctgtacggtaaaacggtagggtacgccgtgacgtatcacgcggagggattcctagtgtgca |
| | | agaccacagacactgtcaaaggagaaagagtctcattccctgtatgcacctacgtcccctcaa |
| | | ccatctgtgatcaaatgactggcatactggcgaccgacatcacaccggaggacgcacagaa |
| | | gttgttagtgggattgaatcagaggatagttgtgaacggaagaacacagcgaaacactaacac |
| | | gatgaagaactatctgcttccgattgtggccgtcgcatttagcaagtgggcgagggaatacaa |
| | | ggcagaccttgatgatgaaaaacctctgggtgtccgagagaggtcacttacttgctgctgcttg |
| | | tgggcatttaaaacgaggaagatgcacaccatgtacaagaaaccagacacccagacaatagt |
| | | gaaggtgccttcagagtttaactcgttcgtcatcccgagcctatggtctacaggcctcgcaatc |
| | | ccagtcagatcacgcattaagatgcttttggccaagaagaccaagcgagagtcaatacctgtt |
| | | ctcgacgcgtcgtcagccagggatgctgaacaagaggagaaggagaggttggaggccga |
| | | gctgactagagaagccttaccacccctcgtccccaccgcgccggcggagacgggagtcgtc |
| | | gacgtcgacgttgaagaactagagtatcacgcaggtgcaggggtcgtggaaacacctcgca |
| | | gcgcgttgaaagtcaccgcacagccgaacggcgtactactaggaaattacgtagttctgtccc |
| | | cgcagaccgtgctcaagagctccaagttggcccccgtgcaccctctagcagagcaggtgaa |
| | | aataataacacataacgggagggccggccgttaccaggtcgacggatatgacggcagggtc |
| | | ctactaccatgtggatcggccattccggtccctgagtttcaagctttgagcgagagcgccacta |
| | | tggtgtacaacgaaagggagttcgtcaacaggaaactataccatattgccgttcacggaccgt |
| | | cgctgaacaccgacgaggagaactacgagaaagtcagagctgaaagaactgacgccgagt |
| | | acgtgttcgacgtagataaaaaatgctgcgtcaagagagaggaagcgtcgggtttggtgttgg |
| | | tgggagagctaaccaaccccccgttccatgaattcgcctacgaagggctgaagatcaggccg |
| | | tcggcaccatataagactacagtagtaggagtctttggggttccgggatcaggcaagtctgcta |
| | | ttattaagagcctcgtgaccaaacacgatctggtcaccagcggcaagaaggagaactgccag |
| | | gaaatagtcaacgacgtgaagaagcaccgcggactggacatccaggcaaaaacagtggac |
| | | tccatcctgctaaacgggtgtcgtcgtgccgtggacatcctatatgtggacgaggctttcgcttg |
| | | ccattccggtactctgctagccctaattgctcttgttaaacctcggagcaaagtggtgttatgcg |
| | | gagaccccaagcaatgcggattcttcaatatgatgcagcttaaggtgaacttcaaccacaacat |
| | | ctgcactgaagtatgtcataaaagtatatccagacgttgcacgcgtccagtcacggccatcgtg |
| | | tctacattgcactacggaggcaagatgcgcacgaccaacccgtgcaacaaacccataatcat |
| | | agacaccacaggacagaccaagcccaagccaggagacatcgtgttaacatgcttccgaggc |
| | | tgggtaaagcagctgcagttggactaccgtggacacgaagtcatgacagcagcagcatctca |
| | | gggcctcacccgcaaaggggtatacgccgtaaggcagaaggtgaatgaaaatcccttgtatg |
| | | cccctgcgtcggagcacgtgaatgtactgctgacgcgcactgaggataggctggtgtggaaa |
| | | acgctggccggcgatccctggattaaggtcctatcaaacattccacagggtaactttacggcc |
| | | acattggaagaatggcaagaagaacacgacaaaataatgaaggtgattgaaggaccggctg |
| | | cgcctgtggacgcgttccagaacaaagcgaacgtgtgttgggcgaaaagcctggtgcctgtc |
| | | ctggacactgccggaatcagattgacagcagaggagtggagcaccataattacagcatttaa |
| | | ggaggacagagcttactctccagtggtggccttgaatgaaatttgcaccaagtactatggagtt |
| | | gacctggacagtggcctgttttctgccccgaaggtgtccctgtattacgagaacaaccactgg |
| | | gataacagacctggtggaaggatgtatggattcaatgccgcaacagctgccaggctggaagc |
| | | tagacataccttcctgaaggggcagtggcatacgggcaagcaggcagttatcgcagaaaga |
| | | aaaatccaaccgctttctgtgctggacaatgtaattcctatcaaccgcaggctgccgcacgccc |
| | | tggtgactgagtacaagacggttaaaggcagtagggttgagtggctggtcaataaagtaaga |
| | | gggtaccacgtcctgctggtgagtgagtacaacctggctttgcctcgacgcagggtcacttgg |
| | | ttgtcaccgctgaatgtcacaggcgccgataggtgctacgacctaagtttaggactgccggct |
| | | gacgccggcaggttcgacttggtctttgtgaacattcacacggaattcagaatccaccactacc |
| | | agcagtgtgtcgaccacgccatgaagctgcagatgcttgggggagatgcgctacgactgcta |
| | | aaacccggcggcagcctcttgatgagagcttacggatacgccgataaaatcagcgaagccgt |
| | | tgtttcctccttaagcagaaagttctcgtctgcaagagtgttgcgcccggattgtgtcaccagca |
| | | atacagaagtgttcttgctgttctccaactttgacaacggaaagagaccctctacgctacacca |
| | | gatgaataccaagctgagtgccgtgtatgccggagaagccatgcacacggccgggtgtgca |
| | | ccatcctacagagttaagagagcagacatagccacgtgcacagaagcggctgtggttaacgc |
| | | agctaacgcccgtggaactgtaggggatggcgtatgcagggccgtggcgaagaaatggcc |
| | | gtcagcctttaagggagaagcaacaccagtgggcacaattaaaacagtcatgtgcggctcgt |
| | | accccgtcatccacgctgtagcgcctaatttctctgccacgactgaagcggaagggaaccgc |
| | | gaattggccgctgtctaccgggcagtggccgccgaagtaaacagactgtcactgagcagcgt |
| | | agccatcccgctgctgtccacaggagtgttcagcggcggaagagataggctgcagcaatcc |
| | | ctcaaccatctattcacagcaatggacgccacggacgctgacgtgaccatctactgcagaga |
| | | caaaagttgggagaagaaaatccaggaagccatagacacgaggacggctgtggagttgctc |
| | | aatgatgacgtggagctgaccacagacttggtgagagtgcacccggacagcagcctggtgg |
| | | gtcgtaagggctacagtaccactgacgggtcgctgtactcgtactttgaaggtacgaaattcaa |
| | | ccaggctgctattgatatggcagagatactgacgttgtggcccagactgcaagaggcaaacg |
| | | aacagatatgcctatacgcgctgggcgaaacaatggacaacatcagatccaaatgtccggtg |
| | | aacgattccgattcatcaacacctcccaggacagtgccctgcctgtgccgctacgcaatgaca |
| | | gcagaacggatcacccgccttaggtcacaccaagttaaaagcatggtggtttgctcatcttttcc |
| | | cctcccgaaataccatgtagatggggtgcagaaggtaaagtgcgagaaggttctcctgttcga |
| | | cccgacggtaccttcagtggttagtccgcggaagtatgccgcatctacgacggaccactcag |
| | | atcggtcgttacgagggtttgacttggactggaccaccgactcgtcttccactgccagcgatac |
| | | catgtcgctacccagtttgcagtcgtgtgacatcgactcgatctacgagccaatggctcccata |
| | | gtagtgacggctgacgtacaccctgaacccgcaggcatcgcggacctggcggcagatgtgc |
| | | atcctgaacccgcagaccatgtggacctcgagaacccgattcctccaccgcgcccgaagag |
| | | agctgcataccttgcctcccgcgcggcggagcgaccggtgccggcgccgagaaagccgac |
| | | gcctgccccaaggactgcgtttaggaacaagctgcctttgacgttcggcgactttgacgagca |
| | | cgaggtcgatgcgttggcctccgggattactttcggagacttcgacgacgtcctgcgactagg |
| | | ccgcgcgggtgcatatattttctcctcggacactggcagcggacatttacaacaaaaatccgtt |
| | | aggcagcacaatctccagtgcgcacaactggatgcggtcgaggaggagaaaatgtacccgc |
| | | caaaattggatactgagagggagaagctgttgctgctgaaaatgcagatgcacccatcggag |
| | | gctaataagagtcgataccagtctcgcaaagtggagaacatgaaagccacggtggtggaca |
| | | ggctcacatcgggggccagattgtacacgggagcggacgtaggccgcataccaacatacg |
| | | cggttcggtacccccgccccgtgtactcccctaccgtgatcgaaagattctcaagccccgatg |
| | | tagcaatcgcagcgtgcaacgaatacctatccagaaattacccaacagtggcgtcgtaccag |
| | | ataacagatgaatacgacgcatacttggacatggttgacgggtcggatagttgcttggacaga |
| | | gcgacattctgcccggcgaagctccggtgctacccgaaacatcatgcgtaccaccagccga |
| | | ctgtacgcagtgccgtcccgtcaccctttcagaacacactacagagcgtgctagcggccgcc |
| | | accaagagaaactgcaacgtcacgcaaatgcgagaactacccaccatggactcggcagtgtt |
| | | caacgtggagtgcttcaagcgctatgcctgctccggagaatattgggaagaatatgctaaaca |
| | | acctatccggataaccactgagaacatcactacctatgtgaccaaattgaaaggcccgaaagc |
| | | tgctgccttgttcgctaagacccacaacttggttccgctgcaggaggttcccatggacagattc |
| | | acggtcgacatgaaacgagatgtcaaagtcactccagggacgaaacacacagaggaaaga |
| | | cccaaagtccaggtaattcaagcagcggagccattggcgaccgcttacctgtgcggcatcca |
| | | cagggaattagtaaggagactaaatgctgtgttacgccctaacgtgcacacattgtttgatatgt |
| | | cggccgaagactttgacgcgatcatcgcctctcacttccacccaggagacccggttctagaga |
| | | cggacattgcatcattcgacaaaagccaggacgactccttggctcttacaggtttaatgatcctc |
| | | gaagatctaggggtggatcagtacctgctggacttgatcgaggcagcctttggggaaatatcc |
| | | agctgtcacctaccaactggcacgcgcttcaagttcggagctatgatgaaatcgggcatgtttc |
| | | tgactttgtttattaacactgttttgaacatcaccatagcaagcagggtactggagcagagactc |
| | | actgactccgcctgtgcggccttcatcggcgacgacaacatcgttcacggagtgatctccgac |
| | | aagctgatggcggagaggtgcgcgtcgtgggtcaacatggaggtgaagatcattgacgctgt |
| | | catgggcgaaaaacccccatatttttgtgggggattcatagtttttgacagcgtcacacagacc |
| | | gcctgccgtgtttcagacccacttaagcgcctgttcaagttgggtaagccgctaacagctgaa |
| | | gacaagcaggacgaagacaggcgacgagcactgagtgacgaggttagcaagtggttccgg |
| | | acaggcttgggggccgaactggaggtggcactaacatctaggtataaggtagagggctgca |
| | | aaagtatcctcatagccatggccaccttggcgagggacattaaggcgtttaagaaattgagag |
| | | gacctgttatacacctctacggcggtcctagattggtgcgttaatacacagaattctgattggatc |
| | | cacgatggcacgcgatattgatccttacaaggagttcggggcaaccgtcgagctgctgagctt |
| | | cctgccaagcgacttctttccatcagtgcgcgatctgctggataccgctagcgctctgtaccgg |
| | | gaggctctcgagtccccagagcactctagccctcaccacacagcactgaggcaggccattct |
| | | gtcttggggagagctcatgaccctcgcaacttgggtgggcgtgaacctggaggacccagcat |
| | | ctcgcgacctcgtcgtgtcatacgtgaatacaaatatgggcctgaagtttagacagctgctgtg |
| | | gtttcacatttcaagcctgacattcggacgcgagacagtgattgagtacctggtgtcatttggcg |
| | | tgtggattagaacccctcccgcttacaggcctcctaatgccccaattctgtctactctgcccgag |
| | | actaccgtcgtgcgcagacggggaagatctccacgccggcggactccatcacctagacggc |
| | | gcaggtcccagtctccaagacggcgccggtcacagtctagggagagccagtctgagggaa |
| | | gaggcagcctgctgacctgcggcgacgtggaggagaacccaggcccaatgaagtgcctttt |
| | | gtacttagcctttttattcattggggtgaattgcaagttcaccatagtttttccacacaaccaaaaa |
| | | ggaaactggaaaaatgttccttctaattaccattattgcccgtcaagctcagatttaaattggcat |
| | | gatgacttaataggcacagccttacaagtcaaaatgcccaagagtcacaaggctattcaagca |
| | | gacggttggatgtgtcatgcttccaaatgggtcactacttgtgacttccgctggtatggaccgaa |
| | | gtatataacacattccatccgatccttcactccatctgtagaacaatgcaaggaaagcattgaac |
| | | aaacgaaacaaggaacttggctgaatccaggcttccctcctcaaagttgtggatatgcaactgt |
| | | gacggatgccgaagcagtgattgtccaggtgactcctcaccatgtgctggttgatgaatacac |
| | | aggagaatgggttgattcacagttcatcaacggaaaatgcagcaattacatatgccccactgtc |
| | | cataactctacaacctggcattctgactataaggtcaaagggctatgtgattctaacctcatttcc |
| | | atggacatcaccttcttctcagaggacggagagctatcatccctgggaaaggagggcacagg |
| | | gttcagaagtaactactttgcttatgaaactggaggcaaggcctgcaaaatgcaatactgcaag |
| | | cattggggagtcagactcccatcaggtgtctggttcgagatggctgataaggatctctttgctgc |
| | | agccagattccctgaatgcccagaagggtcaagtatctctgctccatctcagacctcagtggat |
| | | gtaagtctaattcaggacgttgagaggatcttggattattccctctgccaagaaacctggagca |
| | | aaatcagagcgggtcttccaatctctccagtggatctcagctatcttgctcctaaaaacccagg |
| | | aaccggtcctgctttcaccataatcaatggtaccctaaaatactttgagaccagatacatcagag |
| | | tcgatattgctgctccaatcctctcaagaatggtcggaatgatcagtggaactaccacagaaag |
| | | ggaactgtgggatgactgggcaccatatgaagacgtggaaattggacccaatggagttctga |
| | | ggaccagttcaggatataagtttcctttatacatgattggacatggtatgttggactccgatcttca |
| | | tcttagctcaaaggctcaggtgttcgaacatcctcacattcaagacgctgcttcgcaacttcctg |
| | | atgatgagagtttattttttggtgatactgggctatccaaaaatccaatcgagcttgtagaaggtt |
| | | ggttcagtagttggaaaagctctattgcctcttttttctttatcatagggttaatcattggactattctt |
| | | ggttctccgagttggtatccatctttgcattaaattaaagcacaccaagaaaagacagatttatac |
| | | agacatagagatgaaccgacttggaaagtaattaattaataatcatattaagggcccaagggg |
| | | ctgcaaaagtatcctcatagccatggccaccttggcgagggacattaaggcgtttaagaaattg |
| | | agaggacctgttatacacctctacggcggtcctagattggtgcgttaatacacagaattctgatt |
| | | ggcgcgccacgatgcagtggaattccacaaccttccaccaaactctgcaagatcccagagtg |
| | | agaggcctgtatttccctgctggtggctccagttcaggaacagtaaaccctgttctgactactgc |
| | | ctctcccttatcgtcaatcttctcgaggattggggaccctgcgctgaacatggagaacatcacat |
| | | caggattcctaggaccccttctcgtgttacaggcggggtttttcttgttgacaagaatcctcacaa |
| | | taccgcagagtctagactcgtggtggacttctctcaattttctagggggaactaccgtgtgtcttg |
| | | gccaaaattcgcagtccccaacctccaatcactcaccaacctcttgtcctccaacttgtcctggt |
| | | tatcgctggatgtgtctgcggcgttttatcatcttcctcttcatcctgctgctatgcctcatcttcttgt |
| | | tggttcttctggactatcaaggtatgttgcccgtttgtcctctaattccaggatcctcaacaaccag |
| | | cacgggaccatgccggacctgcatgactactgctcaaggaacctctatgtatccctcctgttgc |
| | | tgtaccaaaccttcggacggaaattgcacctgtattcccatcccatcatcctgggctttcggaaa |
| | | attcctatgggagtgggcctcagcccgtttctcctggctcagtttactagtgccatttgttcagtg |
| | | gttcgtagggctttcccccactgtttggctttcagttatatggatgatgtggtattgggggccaag |
| | | tctgtacagcatcttgagtccctttttaccgctgttaccaattttcttttgtctttgggtatacatttaac |
| | | ctgcaggaccataactgtataacttgtaacaaagcgcaacaagacctgcgcaattggccccgt |
| | | ggtccgcctcacggaaactcggggcaactcatattgacacattaattggcaataattggaagct |
| | | tacataagcttaattcgacgaataattggatttttattttattttgcaattggtttttaatatttccaaaa |
| | | aaaaaaaaaaaaaaaaaaaaaaaaaaaaaaaaaaaaaaaaaaaaaaaaaaaaaa |

In various embodiments, vectors or plasmids may comprise and/or encode one or more of SEQ ID NO: 1-15. In various embodiments, vectors or plasmids may comprise and/or encode a sequence or sequence portion having more than 70%, or more than 75%, or more than 80%, or more than 85%, or more than 90%, or more than 95%, or more than 96%, or more than 97%, or more than 98%, or more than 99% homology to one or more of SEQ ID NO: 1-15. In some embodiments, vectors or plasmids may comprise a sequence consisting of one or more of SEQ ID NO: 1-15. Where a vector or plasmid comprises a sequence consisting of or encoding one or more of SEQ ID NO: 1-15, it is intended that the vector or plasmid may comprise additional sequence domains.

In exemplary embodiments, plasmids may have a polynucleotide sequence corresponding to SEQ ID NO: 16 or SEQ ID NO: 17. In exemplary embodiments, plasmids may have a polynucleotide sequence having more than about 70%, or more than 75%, or more than 80%, or more than 85%, or more than 90%, or more than 95%, or more than 96%, or more than 97%, or more than 98%, or more than 99% homology to SEQ ID NO: 16 or SEQ ID NO: 17. In some embodiments, a plasmid may have a polynucleotide sequence consisting of SEQ ID NO: 16 or SEQ ID NO: 17. In some embodiments, a plasmid may have a polynucleotide sequence consisting essentially of SEQ ID NO: 16 or SEQ ID NO: 17. Where a plasmid has a polynucleotide sequence consisting essentially of SEQ ID NO: 16 or SEQ ID NO: 17, it is intended that the plasmid, having the same general sequence domains, may contain one or more nucleotide and/or amino acid substitutions, additions, or deletions in or between those domains which do not significantly impact the function of the plasmid.

Where a sequence “homology” or “identity” is contemplated, for a DNA sequence or an amino acid sequence, the same percentage “similarity” is also contemplated for the amino acid sequence or amino acid sequence corresponding to the DNA sequence. The term “similarity” is different from the term identity because it allows conservative substitutions of amino acid residues having similar physicochemical properties over a defined length of a given alignment. Generally, any reasonable similarity-scoring matrix known may be used to determine similarity.

In determining the sequence homology or identity of a first sequence compared to a second sequence, various identity calculations may be performed such as those implemented in the National Institute of Health's Basic Local Alignment Search Tool (BLAST). In some embodiments, the standard BLAST settings may be utilized. For example, a BLAST identity may be defined as the number of matching bases over the number of alignment positions.

VLVs can generally be produced by transfecting any appropriate cell line with appropriate plasmids or vectors. In an embodiment, VLVs are produced by transfecting BHK-21 or HEK293 T cells with a vector or plasmid, incubating the transfected BHK-21 or HEK293 T cells in a buffer solution for a suitable time and at a suitable temperature to propagate VLVs; and isolating the VLVs from the BHK-21 or HEK293 T cells and buffer solution by a technique selected from the group consisting of ultrafiltration, centrifugation, tangential flow filtration, affinity purification, ion exchange chromatography, and combinations thereof. In various embodiments, VLVs can be produced by any appropriate transduction, incubation, and isolation methods.

The produced VLVs are generally useful for therapeutic methods. The produced VLVs may be formulated as vaccine compositions for treatment of HBV with one or more diluents, excipients, or other ingredients. The compositions may generally be administered by any appropriate route, such as by oral, parenteral, intravenous, or other routes.

The figures are described in more detail as follows, with reference to the Examples presented herein.

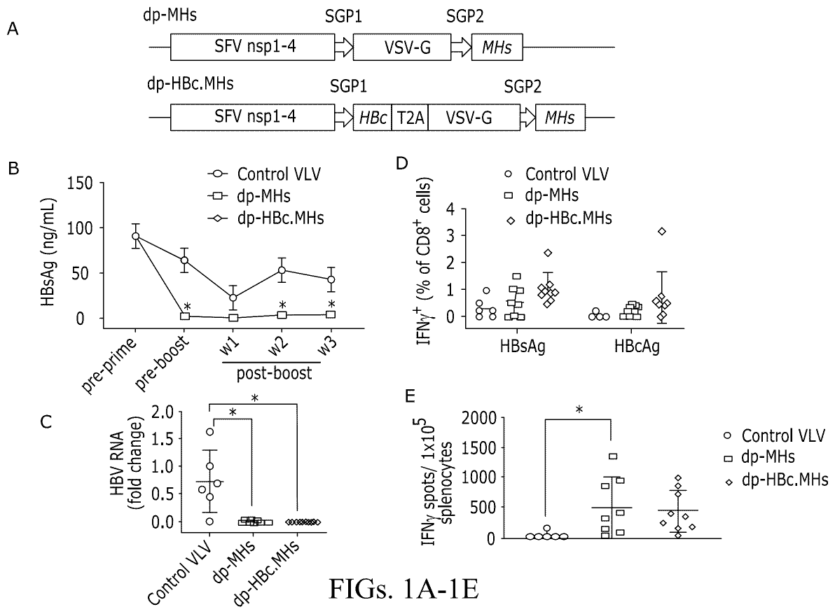

FIG. 1 depicts effects of dp-HBc.MHs (CARG-201) and dp-MHs on HBsAg levels in a chronic AAV-HBV model. FIG. 1A depicts schematics of single-antigen (dp-MHs) and dual-antigen (CARG-201) vectors. Chronic HBV was established in C57BL/6 mice using HBV genome delivery by AAV2/AAV8. Groups were balanced for HBsAg prior to prime immunization with control vector (expressing eGFP, n=6), dp-MHs (expressing HBV middle S antigen, n=8), or CARG-201 (expressing HBV core and middle S antigens, n=9) 6 weeks after AAV-HBV transduction. Animals were boosted 4 weeks later using serotype-switched VLV vectors. FIG. 1B depicts ELISA analysis of HBsAg (ng/mL). FIG. 1C depicts qRT-PCR of liver HBV RNA. FIG. 1D depicts flow cytometry of HBV-specific CD8 T cells using intracellular staining for IFNγ after stimulation with HBsAg or HBcAg peptide pools. FIG. 1E depicts ELISPOT of HBV-specific CD8 T cells using an HBsAg peptide pool. Data are the mean+SEM. Asterisks indicate a significant difference between the control and HBV antigen-expressing VLV (p<0.01). HBc=HBcAg (core); MHs=MHBs (surface); dp=double subgenomic promoter; SGP1/2, sub-genomic promoter 1 or 2.

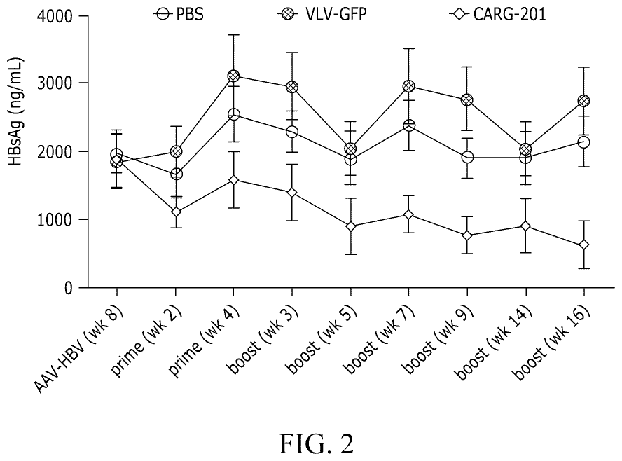

FIG. 2 depicts therapeutic vaccine candidate CARG-201 in prime-boost immunization controls HBV in mice with higher pre-existing HBV antigen levels. To further understand the effect of antigenemia on CARG-201-mediated HBV control, we evaluated anti-HBV efficacy in mice with even higher antigen levels than previously used (100-500 ng/ml). We transduced mice with 1×10¹¹ genome copies of AAV-HBV 1.2-mer, and groups of animals with moderate-high (average HBsAg ˜3,000 ng/ml) levels of HBsAg were selected for the immunization groups. Persistent HBV replication was determined by measuring serum HBsAg levels at 8 weeks post-transduction. Two groups of 12 mice were primed i.m. with 10⁸ PFU/mouse of CARG-201 and VLV-GFP and boosted 4 weeks later (Pre). A significant HBsAg reduction arises for CARG-201 but is not apparent in the GFP or PBS control.

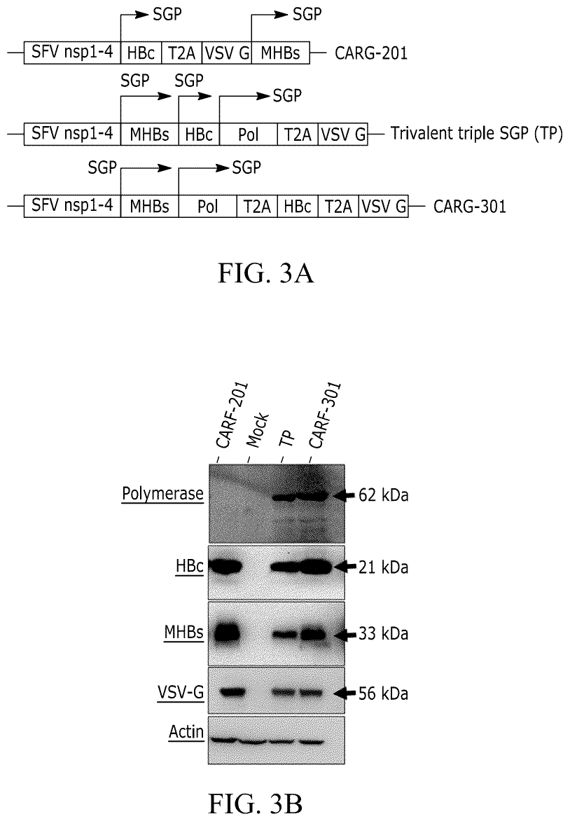

FIG. 3 depicts construction and expression VLV-based recombinant multivalent HBV vaccines. FIG. 3A depicts exemplary chema of CARG-201 and CARG-301candidates. The four non-structural proteins of the Semliki Forest virus (SFV) replicase are designated (nsp 1-4). HBV polymerase (Pol) is deleted of its terminal protein from the four structural domains comprising the enzyme. Expression of Pol and core antigens are fused to the downstream gene by a piconavirus, Thosea asigna virus 2A (T2A) ribosome skipping sites. FIG. 3B depicts expression of HBV genes as assayed by western blot in BHK21 cell lysate. The expression of both 2 and 3 antigens are compared.

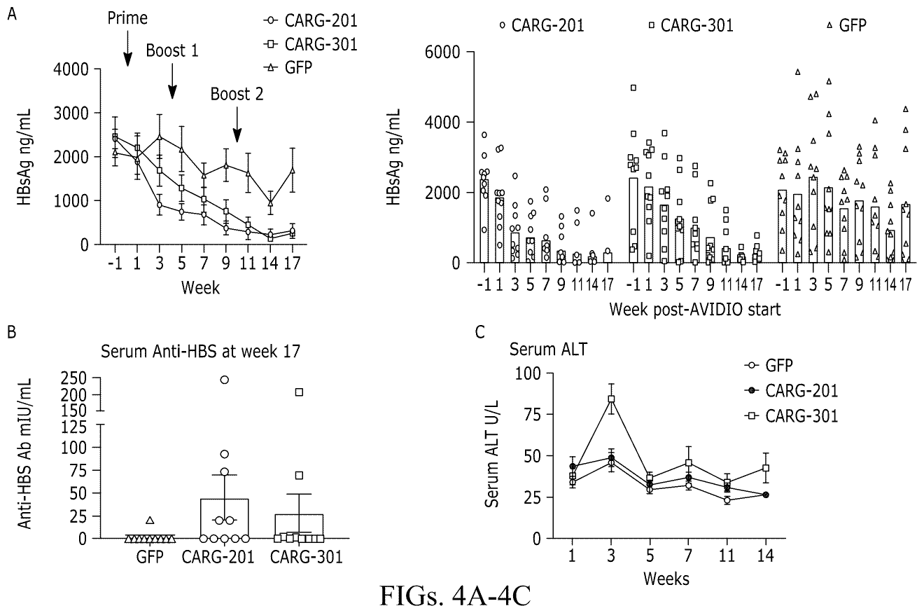

FIG. 4 depicts therapeutic vaccine candidates CARG-201 and CARG-301 in prime-boost immunization controls HBV in mice with high pre-existing HBV antigen levels. CARG-201 harbors two antigens (HBcAg and MHBs) and CARG-301 (HBcAg, MHBs, Polymerase). To further understand the effect of antigenemia on CARG-mediated HBV control, we evaluated anti-HBV efficacy in mice with higher antigen levels as described in FIG. 3. High levels (average HBsAg ˜3,000 ng/ml) of HBsAg were selected for the immunization groups. Persistent HBV replication was determined by measuring serum HBsAg levels at 8 weeks post-transduction. Three groups of 10 mice were primed i.m. with 10⁸ PFU/mouse of CARG-201, CARG-301 and GFP and boosted 4 weeks later (Boost 1) and 6 weeks after the first boost (Boost 2). A significant HBsAg reduction arises for both CARG-201 and CARG-301 but is not apparent in the GFP control in both average values (left panel) and individual values (right panel). Prime, the RNA replicon encoding VSV GNJ serotype; Boost 1, VSV GIN serotype; Boost 2, VSV GCH serotype; NJ=New Jersey; IN=Indiana; CH═Chandipura.

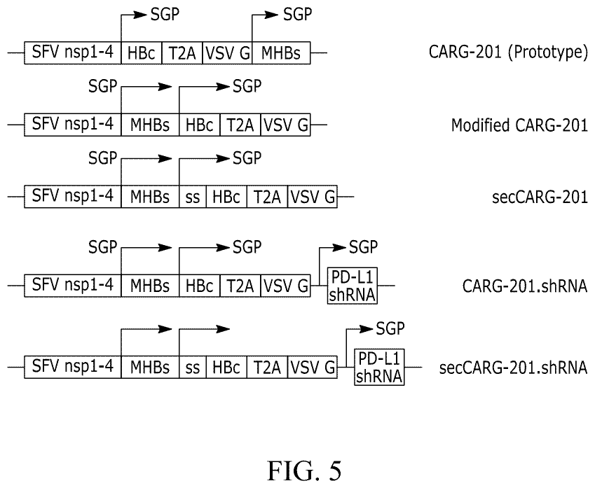

FIG. 5 depicts exemplary schematic depictions of modified CARG-201 vaccine construct for enhanced immunogenicity and efficacy by incorporating secretory signals and shRNA for PD-L1. The non-structural proteins of the Semliki Forest virus (SFV) replicase are designated (nsp1-4). The secretion signal (s.s.) is derived from the VSV G glycoprotein, secretion terminal protein from the four structural domains comprising the enzyme.

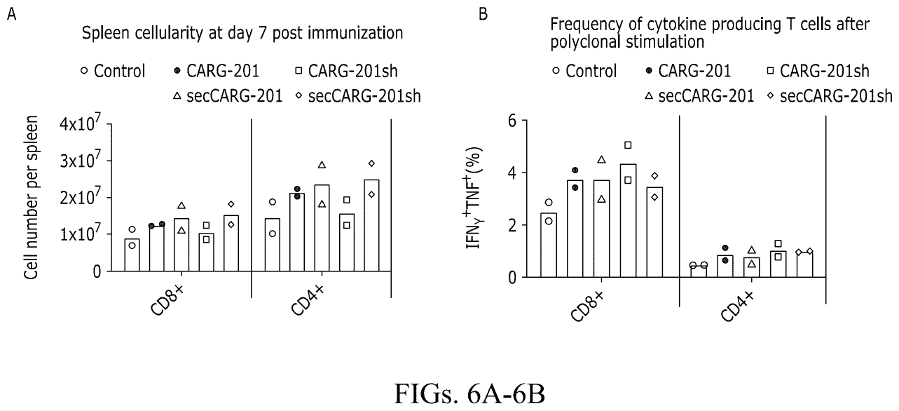

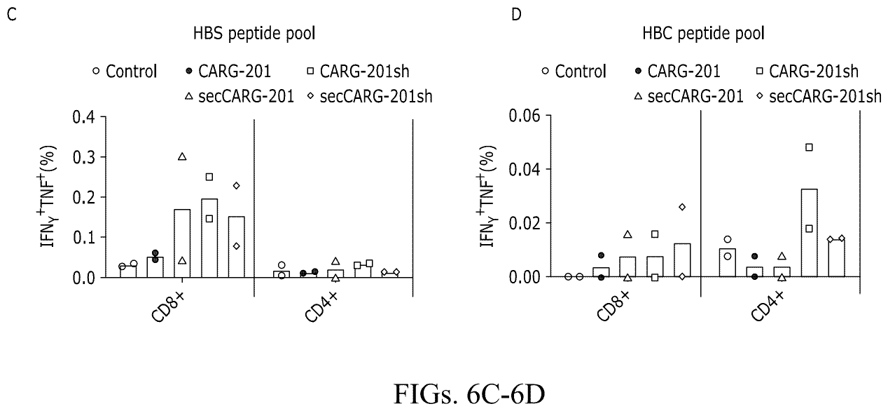

FIG. 6 depicts comparisons of the immunogenicity of modified CARG-201 variants in naïve CB6F1 mice,

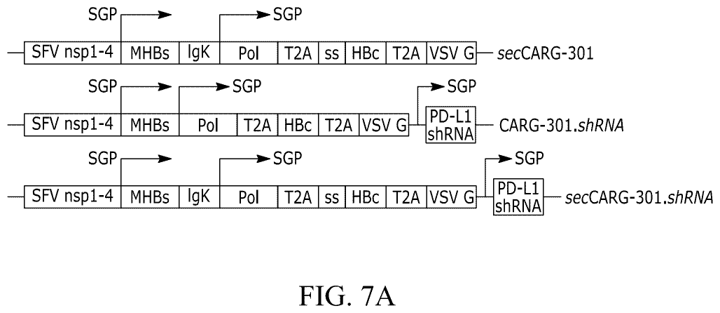

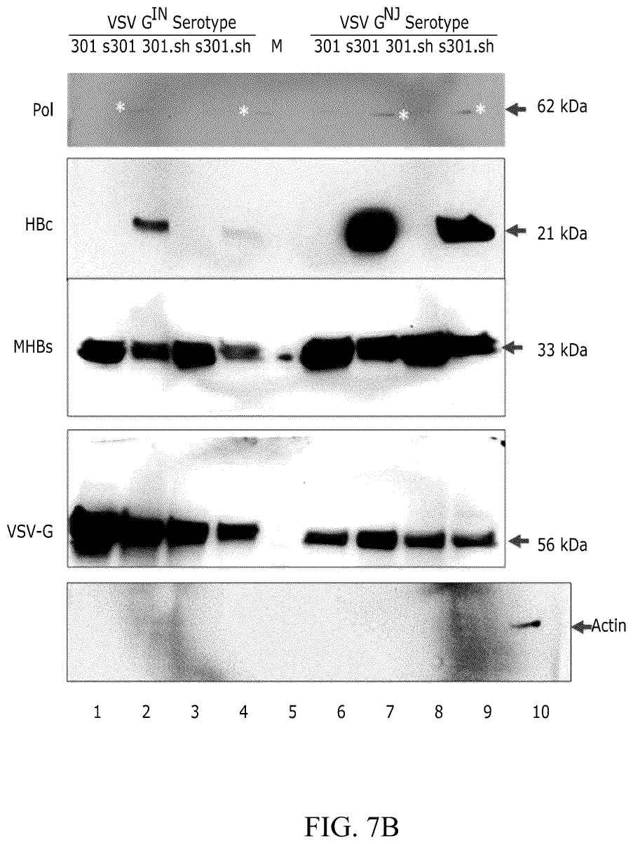

FIG. 7. Depicts expression and secretion of VLV-based recombinant modified CARG-301multivalent HBV vaccines. FIG. 7A depicts exemplary schema of CARG-301 candidate constructs. The four non-structural proteins of the Semliki Forest virus (SFV) replicase are designated (nsp1-4). HBV polymerase (Pol) is deleted of its terminal protein from the four structural domains comprising the enzyme. At its N-terminus is fused the human IgK signal sequence. The middle surface antigen (MHBs) and the core antigen (HBcAg) have the native and heterologous VSV G signal sequence (s.s.) fused to their amino termini respectively. Expression of Pol and core antigens are fused to the downstream gene by a piconavirus, Thosea asigna virus 2A (T2A) ribosomeskipping sites. FIG. 7B depicts expression of HBV genes as assayed by western blot in BHK21 cell lysate. The expression of 3 antigens are compared. Culture supernatants were collected and assayed for the secretion of the HBV antigen. It appears that secreted polymerase is subject to rapid degradation in the culture media as detected by faint bands detected (indicated by asterisks * in white) only after concentration of culture supernatants. Actin protein is detected in the cell lysate (lane 10) but not in the cell supernatants (lanes 1-9). Adding a cocktail of protease inhibitors may inhibit degradation and enhance its detection. HBc, HBcAg; C=control; 301=CARG-301; s301=secCARG-301; 301.sh=CARG-301.shRNA; s301.sh=secCARG-301.shRNA. IN=New Jersey serotype; NJ=New Jersey serotype.

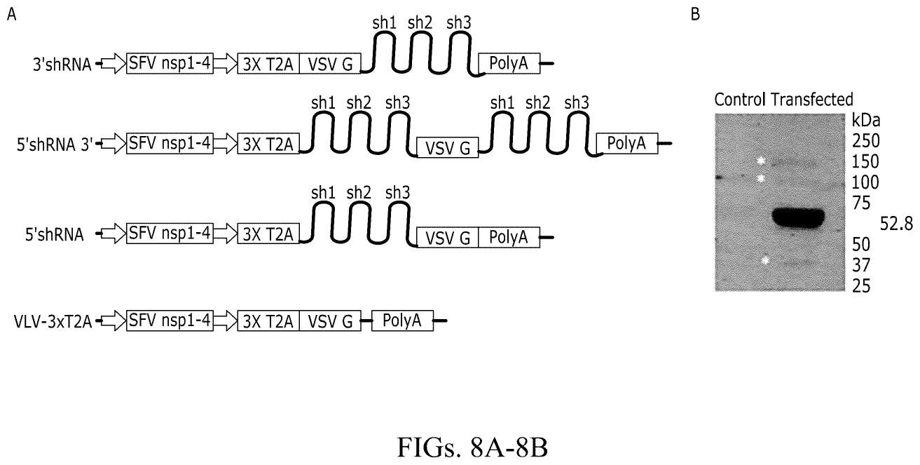

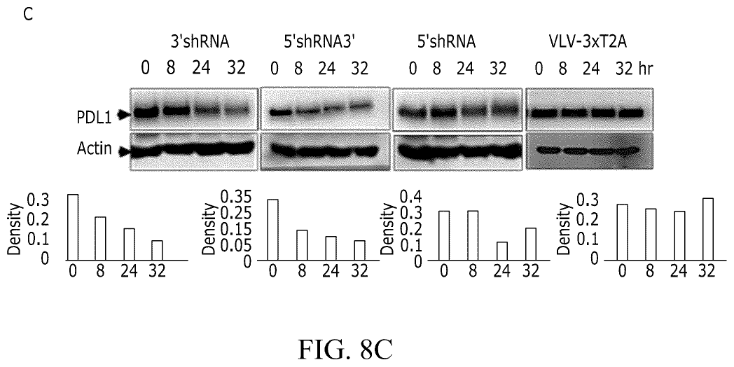

FIG. 8. depicts shRNA inhibits PD-L1 expression in stably transfected BHK21 cells in vitro. FIG. 8A depicts exemplary schema of VLV therapeutic vaccine (VLV-3×T2A) that harbors multiple PD-L1 specific shRNAs 5′ and 3′ of VSV G glycoprotein. Expression of HBV major antigens (MHBs, HBcAg and Polymerase) in VLVs is linked to VSV G by a picornavirus Thosea asigna virus 2A (T2A) ribosome skipping sites. FIG. 8B depicts A mouse cDNA clone of PD-L1, CD274/B7-H1/PD-L1 ORF (Sino biological Inc), was cloned into the mammalian expression vector, pCMV3-Flag-mCD274. BHK21 cells were transfected and hygromycin-resistant clones were selected and amplified. PD-L1 expression in stable cells was analyzed by western blot with anti-PDLI antibodies. FIG. 8C depicts VLVs produced by transfecting BHK21 cells using three versions of shRNA 3XT2A constructs. VLVs were produced and titered, 0.1 MOI was used to infect BHK21: PD-L1 stable cell line. Lysates were prepared by collecting cells after 8, 24 and 32 hours. PDLI downregulation was analyzed by western blots and compared with non-infected cells lysate. Band intensity quantified by GelQuant.NET software (BiochemLabSolutions.com) normalized to β-actin.

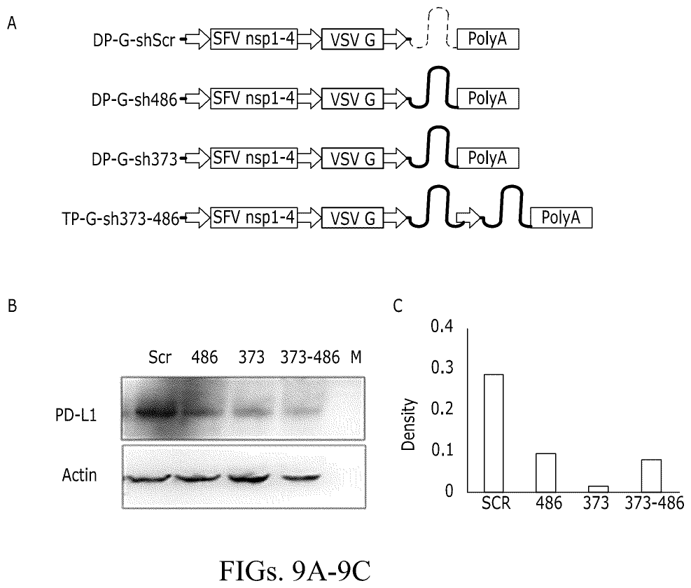

FIG. 9 depicts downregulation of PDLI with shRNA VLV constructs. FIG. 9A depicts exemplary schematic depictions of empty VLV constructs in which shRNA is driven by one or two sub-genomic promoters. FIG. 9B depicts Western blot analysis of stable BHK21 cells constitutively expressing PD-L1. The cell lysates were prepared and analyzed after 6 hours of infection. FIG. 9C depicts densitometric quantification of blot after normalization to actin. Scr; scrambled shRNA; sh373 and sh486; shRNAs at positions relative to PD-L1 sequence (bp).

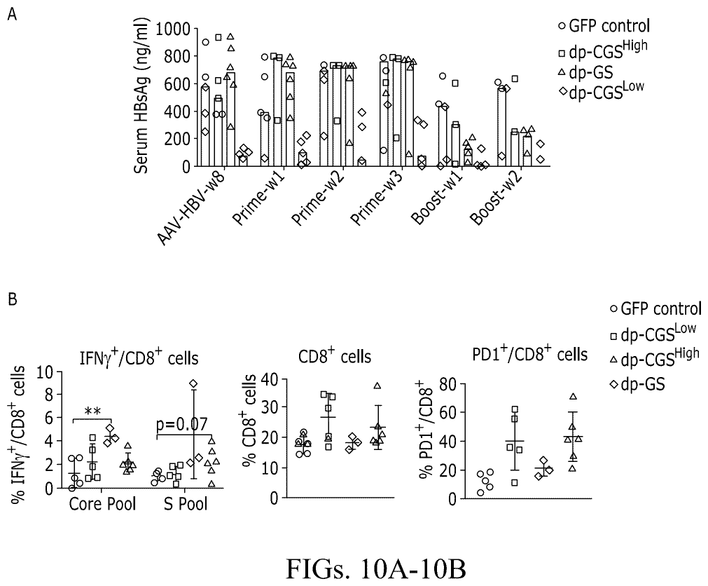

FIG. 10 depicts CARG-201 dramatically reduces serum HBsAg levels and induces core-specific T cells in a more stringent AAV-HBV model HBsAg^{High}). FIG. 10A shows mice were transduced with AAV-HBV1.2-mer and chronicity was fully established by week 8 (wk8) and mice were then segregated into high antigen (HBsAg^{High}) and low antigen (HBsAgLow). Mice were then immunized (primed) with constructs as indicated (Prime-wk1). One month after prime, the mice were boosted with the same construct in which the VSV G glycoprotein from a different serotype was switched. FIG. 10B shows core specific T cells and PD1 cells. Single-antigen (dp-GS) appears as effective as CARG-201 (dp-CGS) for induction of HBV-specific T cells, as measured by ELISPOT. Increasing the HBV viral load increases the induction of core specific T cells after immunization with dp-CGS, as measured by intracellular cytokines staining for IFNγ+ in the CD8⁺ T cells (left panel).

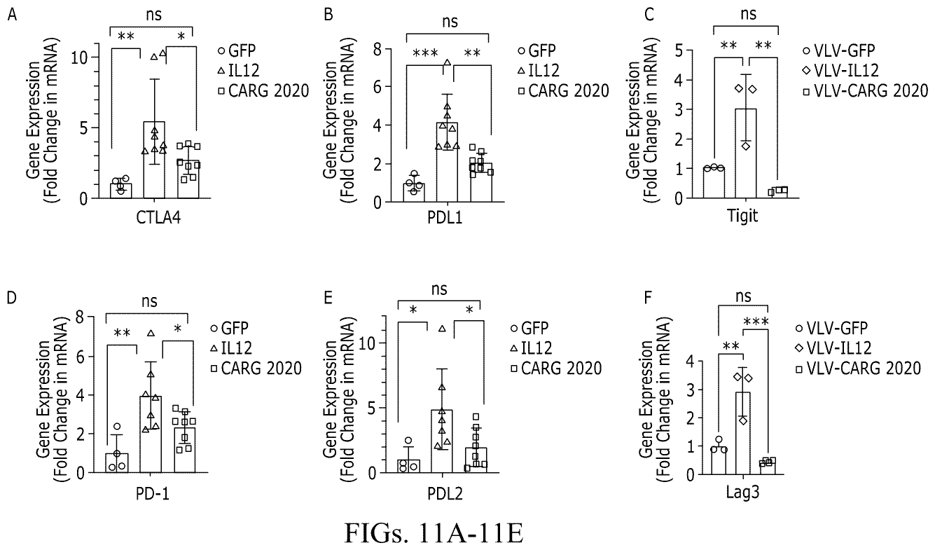

FIG. 11 depicts blockade of PD-1/PD-L1 pathway by shRNA in vivo significantly inhibits expression of immune checkpoints and inhibitory receptors in MC38 tumored-mice. CARG-2020 is a replicon vector harboring three immunomodulators (IL-12, DIL-17RA and shRNA), whereas IL-12 and GFP is a replicon vector expressing rIL-12 (p35 and p40 subunits) and GFP respectively. C57BL/6 mice (n=10) were implanted with MC38 cells and the tumors were injected intratumorally with 5×10\hat{ }7 PFU of the indicated vectors at day 0, 3 and 6. Tumors were harvested when they had significantly regressed in CARG-2020 and IL-12 groups but not in the control GFP group and RNA prepared and analyzed by qPCR using GeneQuery kit (Cat #MGK121) from ScienCell (Carlsbad, CA). Inhibitory receptors are depicted in A and D, receptor ligands in B and E, and other immune checkpoint genes in C and F

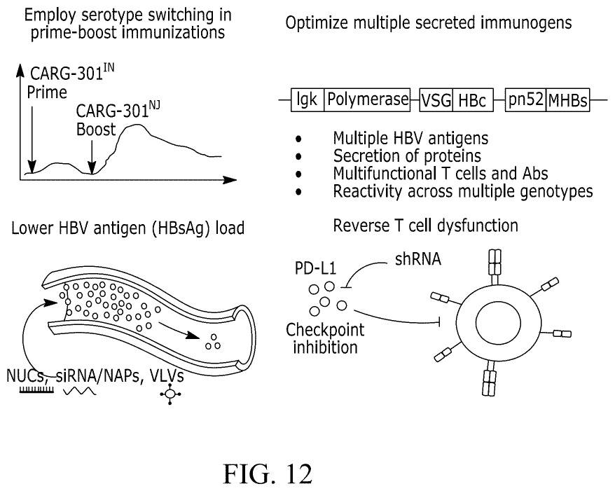

FIG. 12 explores multiple strategies to improve efficacy of therapeutic HBV vaccine in animals and in humans. Strategies include (i) combining vaccine platforms in heterologous prime-boost regimens: (ii) optimization of vaccine immunogen by including multiple HBV antigens incorporating signal sequences; (iii) reversing T cell dysfunction with concomitant inhibition shRNA inhibition of immune checkpoints (iv) immunizing individuals with lower HBV antigen loads by reducing antigenic load prior to vaccination with NUC therapy, siRNA inhibition, nucleic acid polymers (NAPs) or VLVs.

### Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods.

Key points include:
Double-promoter (DP) or triple promoter (TP) VLV platform promotes higher gene expression and enhances immunogenicity
Constructs CARG-201 drive complete clearance of HBsAg in mice with low antigenemia
CARG-201 dramatically reduces serum biomarker levels and induces T cells in stringent mouse model
Both CARG-201 and CARG-301 break tolerance in high antigenemic mice and drive dramatic reduction in HBsAg in mice.
Modification of the CARG-201 with the secreted core antigen or shRNA for PD-L1 increases immunogenicity in naïve mice
shRNA inhibits cells constitutively expressing PD-L1 in vitro. In vivo, a CARG-2020 construct expressing both IL-12 and PD-L1 shRNA down regulates the expression of multiple immune checkpoints in PD-L1+tumor cell line (MC38).
ER-targeting secretion signal sequences enhance secretion HBV antigens in CARG-301
Modified CARG-201 and CARG-301 constructs can be scaled-up and produced.
   We have established that the treatment of mice chronically infected with AAV-HBV with CARG-101 (VLV harboring MHBs, only one HBV antigen) significantly reduces and, in some animals, eliminates serum HBsAg, a surrogate biomarker for viral persistence in the liver. The clearance of HBsAg in 40% of the AAV-HBV mice by only one immunization with CARG-101 is a superior outcome compared with other HBV immunotherapies being developed in the same animal model.

Remarkably, we have now attained a reduction in serum biomarker levels in >80% AAV-HBV mice (n=10) with high antigenemia, indicating that CARG-201 (HBcAg and MHBs: two HBV antigens) can break tolerance in highly tolerant models. To attain this reduction, we combined an enhanced gene expression strategy and a robust prime-boost regimen to achieve complete clearance of HBsAg in mice. We have therefore selected CARG-201 for advancement to the clinic based on the following results: (i) complete reduction of HBsAg in most of but not all treated animals in a mouse model of persistent HBV replication, (ii) reduction of HBV RNA in the liver to undetectable levels, and (iii) induction of multi-specific HBV T cells and antibodies. The reduction in intrahepatic HBV RNA may be the result of strong immune control under a high level of CD8⁺ and CD4⁺ T-cell responses, as observed in patients with resolution of acute HBV infection (FIGS. 1 and 2).

### CARG-201 is Delivering Transgenes for Two HBV Antigens (MHBs and HBc):

Enables robust expression and secretion of HBV middle S and core antigens in vitro
Induces broad immune responses.
Results in reduction HBV marker surface antigens by more than 2 logs in AAV model
Eliminates virus as monotherapy or in combination with standard antiviral therapy
   We reasoned that incorporation of a third antigen such as the polymerase (Pol) combined with a prime-boost immunization might generate a stronger, multi-specific and multi-functional T-cell response that will ultimately control the virus in 100% of the infected mice (FIG. 3). We therefore designed CARG-301 harboring MHBs, HBcAg plus Polymerase (three HBV antigens) and showed that it clears the virus with similar efficacy and kinetics as CARG-201 (FIG. 4). In some embodiments, the MHBs may be any known MHBs. In some embodiments, the HBcAg may be SEQ ID NO: 10 (DNA), SEQ ID NO: 9 (amino acid). In some embodiments, the polymerase may be SEQ ID NO: 12 (DNA), SEQ ID NO: 10 (amino acid).

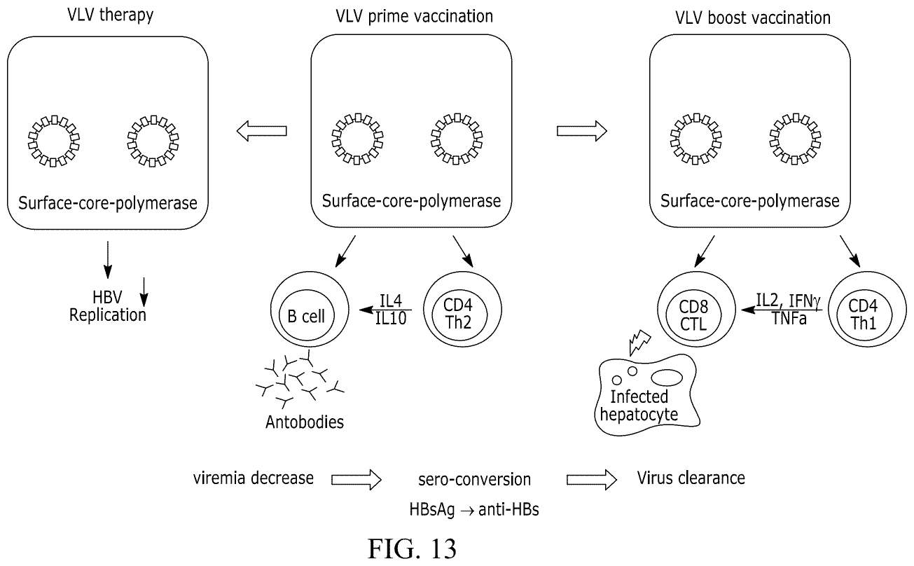

FIG. 13 an exemplary understanding of how HBV immunotherapy works to achieve a functional cure with either CARG-201 or CARG-301. The genome of this partially double-stranded DNA hepatotropic virus uses four overlapping open reading frames to encode seven proteins—core (HBcAg), surface [large (L), middle (M), and small(S) HBs], HBcAg, polymerase, and HBx. It is generally believed that broad multi-specific immune responses would be most beneficial. Therefore, in addition to evaluating the efficacy of a single antigen we have tested the efficacy of multiple antigens delivered on a single vector. The multiple antigen approaches disclosed herein have several advantages over single antigen constructs, as described herein.

FIG. 13 depicts exemplary rationales for development of optimized VLV candidate: a paradigm for therapeutic vaccine (immunotherapy) against HBV. Therapeutic vaccines are currently being developed for multiple chronic viral infections such as HIV, HCV, HPV and HSV. As an alternative to antiviral treatment or to support only partially effective standard HBV therapy, the VLV is designed to activate the patient's immune system to fight and finally control or ideally even eliminate the virus. Whereas the success of prophylactic vaccination is based on rapid neutralization of the invading pathogen by antibodies, virus control and elimination of infected cells require T cells. Therefore, induction of a multi-antigen-specific and multifunctional T-cell response against key viral antigens is a paradigm of therapeutic vaccination-besides activation of a humoral immune response to limit virus spread. Cell-mediated immunity, (CARG-201 or CARG-301) inhibits HBV replication and thereby efficiently reduces viremia. HBV surface antigen (MHBs), core (HBcAg) or Polymerase (Pol) antigens in a prime vaccination stimulates HBV-specific CD4T cell help leading to antibody production by HBV-specific B cells. This results in the production of antigen neutralizing antibodies and ideally in seroconversion from HBsAg to anti-HBs. The vaccination also induces CD8 CTL able to kill infected hepatocytes finally resulting in virus clearance. HBsAg, hepatitis B surface antigen; anti-HBS, antibodies against HBsAg.

These data we have generated establish that (i) we can enhance the antigenic load with a concomitant increase in immunogenicity by modifying the VLV to harbor two or more subgenomic promoters and (ii) the VSV G serotype switch is an effective prime-boost strategy. An optimized single-antigen (MHBs) or double-antigen (MHBs and HBc) vector can drive complete clearance of HBsAg in mice (FIG. 1). The data establish that the reduction in intrahepatic HBV RNA may be due to strong immune control under a high level of CD8+ and CD4⁺ T-cell responses, as in patients with resolution of acute HBV infection. CARG-201 drives HBsAg clearance in highly antigenemic mice (FIG. 2). The data establish that CARG-201 can reduce serum biomarker levels in >80% AAV-HBV mice with high antigenemia. An optimized double boost can drive complete drive complete clearance in highly antigenemic mice (FIG. 3). The data also establish that serotype switch is highly effective prime-boost regimen to significantly reduce (by >1 log) the serum biomarker levels in >80% AAV-HBV mice with high antigenemia. The RNA replicon-based HBV therapeutic vaccine under development can induce CD8+ T cells to multiple antigenic epitopes in the tolerogenic environment of CHB infection, addressing the need for HBV immunotherapy. Further genetic manipulation of either CARG-201 or CARG-301 will drive down further the biomarker levels to >2-3 logs.

### Modifications to the VLV and Antigen Design (FIGS. 5 and 7):

Addition of secretion signal to the Core antigen in CARG-201 to enhance antigen expression. In some embodiments, the secretion signal may be a VSV G secretion signal (for example, SEQ ID NO: 6 (DNA), SEQ ID NO: 5 (amino acid)), or a human IgK secretion signal (for example, SEQ ID NO: 8 (DNA), SEQ ID NO: 7 (amino acid)).
Addition of secretion signals to the Core and Polymerase antigens in CARG-301. In some embodiments, the secretion signal may be a VSV G secretion signal (for example, SEQ ID NO: 6 (DNA), SEQ ID NO: 5 (amino acid)), or a human IgK secretion signal (for example, SEQ ID NO: 8 (DNA), SEQ ID NO: 7 (amino acid)).
Incorporation of the PD-L1 shRNA cassette from CARG-2020 into (VLV harboring IL-12+11-17R+PD-L1 shRNA) to CARG-201 and CARG-301 to increase immunogenicity and overcome immune exhaustion and/or tolerance. In some embodiments, the shRNA may correspond to SEQ ID NO: 13).

As seen in FIG. 6, modification of the CARG-201 with either secreted core antigen or with shRNA for PD-L1 or both in general increases immunogenicity in naïve mice suggesting that efficacy of these vaccines may be enhanced in chronic model of HBV. The ability to modify CARG-301 and to express these variant constructs in vitro makes it eminently possible to utilize these constructs for immunogenicity and efficacy in animal models (FIG. 7).

shRNA inhibits PD-L1 expression in stably transfected BHK21 cells in vitro (FIG. 8). The incomplete inhibition of PD-L1 by shRNA in BHK-21 cells is likely due to steric hindrance generated by closely juxtaposing three RNA loop structures. These structures simultaneously interfere with transcription and inhibit the shRNA transcript from being properly processed into functional siRNA by Dicer. To overcome this problem, we incorporated either one copy or two copies of shRNA separated and driven by two sub-genomic promoters (FIG. 9). A single copy of PD-L1 specific shRNA is more effective at abrogating expression than two or more copies. Taken together, these data indicate that shRNA delivery by VLVs can inhibit PD-L1 expression in vitro suggesting that it is possible to block PD-1/PD-L1 interactions in vivo. The delivery of shRNA carried on VLV-CARG-101 (3×T2A) to block PD-L1 expression generates the exciting possibility that the anti-PD-L1 shRNA combined with immunotherapy is an excellent therapeutic strategy for the treatment of CHB infection.

CARG-201-mediated decrease of serum biomarker is correlated with decreasing population of PD-1⁺/CD8⁺ in high HBsAg AAV-HBV chronic model (FIG. 10). We have shown that shRNA delivery by CARG-201 can inhibit PD-L1 expression in vitro suggesting that it is possible to block PD-1/PD-L1 interactions in vivo. The delivery of shRNA carried on CARG-301 to block PD-L1 expression generates the exciting possibility that the anti-PD-L1 shRNA combined with immunotherapy is an excellent therapeutic strategy for the treatment of CHB infection. The fusion of PD-L1 shRNA to CARG-301 is likely to improve CARG-301 efficacy to 100% in the high stringency AAV-HBV efficacy model and to activate HBV-specific exhausted T-cells (FIG. 7).

A CARG-2020 construct expressing both rIL-12 and PD-L1 shRNA down regulates the expression of multiple immune checkpoints (FIG. 11). The shRNA inhibits not only PD-1 ligand (PD-L1 and PD-L2) expression, but also blocks T-cell co-inhibitory receptors PD-1, CTLA-4, LAG-3 and TIGIT. These immune receptors expressed on activated or exhausted T cells dampen T-cell effector function via diverse inhibitory signaling pathways. Therefore, HBV immunotherapy targeting both PD-1 ligands simultaneously as well as other redundant signaling pathways such as CTLA4 and LAG-3 may provide a clinical benefit by increasing the therapeutic efficacy. Work in mouse models and other mechanistic studies indicate that these approaches may act complementarily and may thus increase therapeutic efficacy. It is therefore highly likely that the incorporation of shRNA into CARG-301 as well as the ability to secrete the antigens will dramatically improve he the immunogenicity and efficacy of CARG-301 in vivo.

### CARG-201 Delivers Transgenes for Two HBV Antigens (MHBs and HBc):

Enables robust expression and secretion of HBV middle S and core antigens in vitro
Induces broad immune responses
Results in reduction HBV marker surface antigens by more than 2 logs in AAV model
Eliminates virus as monotherapy or in combination with standard antiviral therapy

### Modifications of CARG-201 and CARG-301 Antigen Design:

Addition of secretion signal to the Core antigen in CARG-201 to enhance antigen expression
Addition of secretion signals to the Core and Polymerase antigens in CARG-301
Incorporation of the PD-L1 shRNA cassette from CARG-2020 to CARG-201 and CARG-301 to increase immunogenicity and overcome immune exhaustion and/or tolerance.
Modification of the CARG-201 with the secreted core antigen or shRNA for PD-L1 seem to increase immunogenicity in naïve mice as evidenced by increased frequency of HBV-specific T cells.
There are no apparent additive or synergistic effects of the modifications in CARG-201

As seen in Table 2, we have completed the generation of CARG-301 secreting all three antigens (secCARG-301). We have also engineered CARG-301 to incorporate shRNA alone (CARG-301.sh) or both secretion signals and shRNA (secCARG-301.shNA). We will now test the immunogenicity of these constructs and prioritize them for efficacy studies in a chronic mouse model of HBV infection. The availability of constructs in both serotypes will allow us to employ a prime boost regimen if necessary.

TABLE 2
SCALE UP AND PRODUCTION OF VLVS

| Modified CARG-201 and | |
|---|---|
| CARG-301 Variants | Titer (PFU/mL) |
| CARG-201 | 1.2 × 10¹⁰ |
| secCARG-201 | 9 × 10⁹ |
| CARG-201shRNA | 5.75 × 10⁹ |
| secCARG-201shRNA | 2.28 × 10¹⁰ |
| CARG-301 | 8.5 × 10⁹ |
| secCARG-301 | 2.0 × 10⁹ |
| CARG-301shRNA | TBD |
| secCARG-301shRNA | TBD |

### REFERENCES

1. Rolls M M et al. 1994. Novel infectious particles generated by expression of the vesicular stomatitis virus glycoprotein from a self-replicating RNA. Cell 79:497-506.
2. Rose N F et al 2008. Hybrid alphavirus-rhabdovirus propagating replicon particles are versatile and potent vaccine vectors. Proceedings of the National Academy of Sciences of the United States of America 105:5839-5843.
3. Rose N F et al. 2014. In vitro evolution of high-titer, virus-like vesicles containing a single structural protein. Proceedings of the National Academy of Sciences of the United States of America 111:16866-16871.
4. Reynolds T D et al. 2015. Virus-Like Vesicle-Based Therapeutic Vaccine Vectors for Chronic Hepatitis B Virus Infection. J Virol. 89:10407-15
5. Yarovinsky T O et al 2019. Virus-like Vesicles Expressing Multiple Antigens for Immunotherapy of CHB. iScience. 2019. 21:391-402.
6. Chiale C et al. 2020. Modified alphavirus-vesiculovirus hybrid vaccine vectors for homologous prime-boost Immunotherapy of Chronic hepatitis virus 2020. Vaccines 2020; 8:279
7. Balsitis S et al. 2018. Safety and efficacy of anti-PD-L1 therapy in the woodchuck model of HBV infection. PLOS One. 13 (2): e0190058.
8. Jacobi F J. et al. 2019. OX40 stimulation and PD-L1 blockade synergistically augment HBV-specific CD4 T cells in patients with HBeAg-negative infection. J Hepatol. 70:1103-1113.
9. Wykes M N, Lewin S R. 2018. Immune checkpoint blockade in infectious diseases. Nat Rev Immunol. 18:91-104.
10. Dyck L, Mills K H G.2017. Immune checkpoints and their inhibition in cancer and infectious diseases. Eur J Immunol. 47:765-779.
11. Lavanchy D. 2004. Hepatitis B virus epidemiology, disease burden, treatment, and current and emerging prevention and control measures. J Viral Hepat. 2004. 11:97-107
12. Michel M L, Deng Q, Mancini-Bourgine M. 2011. Therapeutic vaccines and immune-based therapies for the treatment of chronic hepatitis B: perspectives and challenges. J Hepatol. 54:1286-96.
13. Beasley R P. Hepatitis B virus. 1988. The major etiology of hepatocellular carcinoma. Cancer. 61 (10): 1942-56.
14. Bosch F X, Ribes J, Cléries R, Díaz M. 2005. Epidemiology of hepatocellular carcinoma. Clin Liver Dis. 9 (2): 191-211
15. European Association for the Study of the Liver. EASL 2017 Clinical Practice Guidelines on the management of hepatitis B virus infection. J Hepatol. 67:370-398.
16. Bertoletti A, Kennedy P T. 2015. The immune tolerant phase of chronic HBV infection: new perspectives on an old concept. Cell Mol Immunol 12:258-63.
17. Gehring A J, Ann D'Angelo J. 2015. Dissecting the dendritic cell controversy in chronic hepatitis B virus infection. Cell Mol; 12:283-91.
18. Kondo Y, Shimosegawa T.2015. Significant roles of regulatory T cells and myeloid derived suppressor cells in hepatitis B virus persistent infection and hepatitis B virus-related HCCs. Int J Mol Sci 16:3307-22.
19. Sjogren M H. 2005. Prevention of hepatitis B in non-responders to initial hepatitis B virus vaccination. Am J Med 118 Suppl 10A: 34S-39S.
20. Poland G A, Jacobson R M. 2004. Clinical practice: prevention of hepatitis B with the hepatitis B vaccine. N Engl J Med 351:2832-2838.
21. Mast E E, Margolis H S, Fiore A E, Brink E W, Goldstein S T, Wang S A, Moyer L A, Bell B P, Alter M J. 2005. A comprehensive immunization strategy to eliminate transmission of hepatitis B virus infection in the United States: recommendations of the Advisory Committee on Immunization Practices (ACIP) part 1: immunization of infants, children, and adolescents. MMWR Recomm Rep 54:1-31
22. Dienstag J L. 2008 Hepatitis B virus infection. N Engl J Med. 359): 1486-500. Erratum in: N Engl J Med. 2010; 363 (3): 298.
23. Zoulim F, Locarnini S. 2009. Hepatitis B virus resistance to nucleos (t) ide analogues. Gastroenterology. 1371593-608.
24. Petersen J, Dandri M, Mier W, et al. 2008. Prevention of hepatitis B virus infection in vivo by entry inhibitors derived from the large envelope protein. Nat Biotechnol. 26:335-41
25. Stray S J, Bourne C R, Punna S, et al. 2005. A heteroaryldihydropyrimidine activates and can misdirect hepatitis B virus capsid assembly. Proc Natl Acad Sci USA. 102:8138-43.
26. Bian Y, Zhang Z, Sun Z, et al. 2017. Vaccines targeting preS1 domain overcome immune tolerance in hepatitis B virus carrier mice. Hepatology 66:1067-82.
27. Zhang T Y, Yuan Q, Zhao J H, et al. 2016. Prolonged suppression of HBV in mice by a novel antibody that targets a unique epitope on hepatitis B surface antigen. Gut 65:658-71.
28. Zhu D, Liu L, Yang D, et al. 2016 Clearing Persistent Extracellular Antigen of Hepatitis B Virus: An Immunomodulatory Strategy To Reverse Tolerance for an Effective Therapeutic Vaccination. J Immunol 196:3079-87.
29. Lazar C, Durantel D, Macovei A, et al. 2017. Treatment of hepatitis B virus-infected cells with alpha-glucosidase inhibitors results in production of virions with altered molecular composition and infectivity. Antiviral Res 76:30-7.
30. Shih C, Chou S F, Yang C C, et al. 2016. Control and Eradication Strategies of Hepatitis B Virus. Trends Microbiol 24:739-49.
31. Isogawa M, Robek M D, Furuichi Y, et al. 2005. Toll-like receptor signaling inhibits hepatitis B virus replication in vivo. J Virol 79:7269-72.
32. Janssen H L A, Brunetto M R, Kim Y J, et al. 2018. Safety, efficacy and pharmacodynamics of vesatolimod (GS-9620) in virally suppressed patients with chronic hepatitis B. J Hepatol; 68:431-40.
33. Wang X, Dong A, Xiao J, et al. 2016. Overcoming HBV immune tolerance to eliminate HBsAg-positive hepatocytes via pre-administration of GM-CSF as a novel adjuvant for a hepatitis B vaccine in HBV transgenic mice. Cell Mol Immunol 13:850-61.
34. Zeng Z, Kong X, Li F, et al. 2013. IL-12-based vaccination therapy reverses liver-induced systemic tolerance in a mouse model of hepatitis B virus carrier. J Immunol 191:4184-93.
35. Fisicaro P, Valdatta C, Massari M, et al. 2010. Antiviral intrahepatic T-cell responses can be restored by blocking programmed death-1 pathway in chronic hepatitis B. Gastroenterology 138:682-93.
36. Xu D Z, Wang X Y, Shen X L, et al. 2013. Results of a phase III clinical trial with an HBsAgHBIG immunogenic complex therapeutic vaccine for chronic hepatitis B patients: experiences and findings. J Hepatol 2013; 59:450-6.
37. Lok A S, Pan C Q, Han S H, et al.2016. Randomized phase II study of GS-4774 as a therapeutic vaccine in virally suppressed patients with chronic hepatitis B. J Hepatol 65:509-16.
38. Martin P, Dubois C, Jacquier E, et al. 2015. TG1050, an immunotherapeutic to treat chronic hepatitis B, induces robust T cells and exerts an antiviral effect in HBV-persistent mice. Gut 64:1961-71.
39. Scott-Algara D, Mancini-Bourgine M, Fontaine H, et al. 2010. Changes to the natural killer cell repertoire after therapeutic hepatitis B DNA vaccination. PLOS One 5: e8761
40. Bertoletti A, Ferrari C, Fiaccadori F, et al. 1991. HLA class I-restricted human cytotoxic T cells recognize endogenously synthesized hepatitis B virus nucleocapsid antigen. Proc Natl Acad Sci USA. 88:10445-9.
41. Chisari F V, Ferrari C. 1988. Hepatitis B virus immunopathogenesis. Annu Rev Immunol. 13:29-60.
42. Nayersina R, Fowler P, Guilhot S, Missale G, Cerny A, Schlicht H J, Vitiello A, Chesnut R, Person J L, Redeker A G, Chisari F V. HLA A2 restricted cytotoxic T lymphocyte responses to multiple hepatitis B surface antigen epitopes during hepatitis B virus infection. J Immunol. 1993 May 15; 150 (10): 4659-71. PMID: 7683326
43. Rehermann B, Fowler P, Sidney J, et.al.1995. The cytotoxic T lymphocyte response to multiple hepatitis B virus polymerase epitopes during and after acute viral hepatitis. J Exp Med. 181:1047-58.
44. Pol S, Nalpas B, Driss F, et al. 2001. Efficacy and limitations of a specific immunotherapy in chronic hepatitis B. J Hepatol. 34:917-21.
45. Jung M-C, Grüner N, Zachoval R, et al. 2002. Immunological monitoring during therapeutic vaccination as a prerequisite for the design of new effective therapies: induction of a vaccine-specific CD4+ T-cell proliferative response in chronic hepatitis B carriers. Vaccine. 20 3598-612.
46. Safadi R, Israeli E, Papo O, Shibolet O, et al. 2003. Treatment of chronic hepatitis B virus infection via oral immune regulation toward hepatitis B virus proteins. Am J Gastroenterol. 98:2505-15.
47. Ren F, Hino K, Yamaguchi Y, et al. 2003. Cytokine-dependent anti-viral role of CD4-positive T cells in therapeutic vaccination against chronic hepatitis B viral infection. J Med Virol. 71:376-84.
48. Horiike N, Fazle Akbar S M, Michitaka K, et al. 2005. In vivo immunization by vaccine therapy following virus suppression by lamivudine: a novel approach for treating patients with chronic hepatitis B. J Clin Virol. 32:156-61.;
49. Lok A S, Zoulim F, Dusheiko G, et al. 2107. Hepatitis B cure: from discovery to regulatory approval. J Hepatol. 2017; 67:847-61.
50. Ning Q, Wu D I, Wang G-Q et al. 2019. Roadmap to functional cure of chronic hepatitis B: an expert consensus. J Viral Hepat. 26:1146-55.
51. Rehermann B, Ferrari C, Pasquinelli C, et al. 1996. The hepatitis B virus persists for decades after patients' recovery from acute viral hepatitis despite active maintenance of a cytotoxic T-lymphocyte response. Nat Med. 2:1104-8.
52. Thimme R, Wieland S, Steiger C, et al. 2003. CD8 (+) T cells mediate viral clearance and disease pathogenesis during acute hepatitis B virus infection. J Virol. 77:68-76.
53. Boni C, Penna A, Bertoletti A, Lamonaca V, Rapti I, Missale G, et al. Transient restoration of anti-viral T cell responses induced by lamivudine therapy in chronic hepatitis B. J Hepatol. 39:595-605.
54. Lok A S, Zoulim F, Dusheiko G et al.2020. Durability of hepatitis B surface antigen loss with nucleotide analogue and peginterferon therapy in patients with chronic hepatitis B. Hepatol Commun. 4:8-20.
55. Ferrari C, Penna A, Bertoletti A, et al. 1990. Cellular immune response to hepatitis B virus-encoded antigens in acute and chronic hepatitis B virus infection. J Immunol. 145:3442-9
56. Jung M C. Hartmann B, Gerlach J T, et al. 1999. Virus-specific lymphokine production differs quantitatively but not qualitatively in acute and chronic hepatitis B infection. Virology. 261:165-72.
57. Vandepapelière P, Lau G K, Leroux-Roels G, et al. 2007. Therapeutic HBV Vaccine Group of Investigators. Therapeutic vaccination of chronic hepatitis B patients with virus suppression by antiviral therapy: a randomized, controlled study of co-administration of HBsAg/AS02 candidate vaccine and lamivudine. Vaccine. 25 8585-97.
58. Evans A A, Fine M, London W T. 1997. Spontaneous seroconversion in hepatitis B e antigen-positive chronic hepatitis B: implications for interferon therapy. J Infect Dis. 176 (4): 845-50.
59. Barber D L, Wherry E J, Masopust D, Zhu B, Allison J P, et al. 2006 Restoring function in exhausted CD8 T cells during chronic viral infection. Nature 439:682-687.
60. Maier H, Isogawa M, Freeman G J, Chisari F V. 2007. PD-1: PD-L1 interactions contribute to the functional suppression of virus-specific CD8+T lymphocytes in the liver. J Immunol 178:2714-2720.
61. Velu V, Titanji K, Zhu B, Husain S, Pladevega A, et al. 2009. Enhancing SIV specific immunity in vivo by PD-1 blockade. Nature 458:206-210.
62. Jacobi F J, Wild K, Smits M, et al. 2019. OX40 stimulation and PD-L1 blockade synergistically augment HBV-specific CD4 T cells in patients with HBeAg-negative infection. J Hepatol. 70 (6): 1103-1113.
63. Penaloza-MacMaster P, Provine N M, Blass E et al. CD4 T Cell Depletion Substantially Augments the Rescue Potential of PD-L1 Blockade for Deeply Exhausted CD8 T Cells. J Immunol. 195:1054-63.
64. Fisicaro P, Valdatta C, Massari M, et al. 2012. Combined blockade of programmed death-1 and activation of CD137 increase responses of human liver T cells against HBV, but not HCV. Gastroenterology. 143:1576-1585.
65. Xu-Monette Z Y, Zhang M, Li J, Young K H. 2017. PD-1/PD-L1 Blockade: Have We Found the Key to Unleash the Antitumor Immune Response? Front Immunol. 2017 8:1597.
66. Wykes M N, Lewin S R. 2018.Immune checkpoint blockade in infectious diseases. Nat Rev Immunol. 2018 18 (2): 91-104.
67. Schell J B. Rose N F. Bahl K. et al. 2011. Significant protection against high-dose simian immunodeficiency virus challenge conferred by a new prime-boost vaccine regimen. J Virol. 85:5764-72.
68. Finkelshtein D, Werman A, Novick D, et al. 2013. LDL receptor and its family members serve as the cellular receptors for vesicular stomatitis virus. Proc Natl Acad Sci USA 110:7306-731
69. van den Pol A N, Mao G, Chattopadhyay A, Rose J K, Davis J N. 2017. Chikungunya, Influenza, Nipah, and Semliki Forest Chimeric Viruses with Vesicular Stomatitis Virus: Actions in the Brain. J Virol. 91 (6): e02154-

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the compositions and methods of the present invention, where the term comprises is used with respect to the compositions or recited steps of the methods, it is also contemplated that the compositions and methods consist essentially of, or consist of, the recited compositions or steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, or prior to a further processing step such as drying, binder removal, heating, sintering, etc. It is recognized that certain components can further react or be transformed into new materials.

All percentages and ratios used herein are on a volume (volume/volume) or weight (weight/weight) basis as shown, or otherwise indicated.

What is claimed is:

1. A high-titer hybrid virus vector for treatment, prophylaxis or prevention of hepatitis B virus infections comprising the following operably linked sequence elements:
a) a first DNA sequence comprising a DNA promoter sequence,
b) a second DNA sequence encoding alphavirus non-structural protein polynucleotide sequences,
c) a third DNA sequence encoding at least two alphavirus subgenomic promoters,
d) a fourth DNA sequence comprising at least two sequence domains each independently selected from the group consisting of
i) a sequence domain encoding an HBV antigen, wherein the sequence domain comprises at least one heterologous secretion signal sequence; and
ii) a sequence domain encoding a human short hairpin RNA (shRNA); and
e) a fifth DNA sequence encoding a vesiculovirus glycoprotein.

2. The vector of claim 1 wherein the alphavirus non-structural protein polynucleotide sequence is a semiliki forest virus sequence having at least 70%, or at least 80%, or at least 90%, or at least 95%, or at least 99% homology to SEQ ID NO: 2.

3. The vector of claim 1, wherein the sequence domain encoding the HBV antigen is selected from a hepatitis B core antigen (HBcAg), a hepatitis B surface antigen (HBsAg), polymerase (Pol), and HBx, and combinations thereof.

4. The vector of claim 3, wherein the hepatitis B core antigen (HBcAg) is a cysteine-modified HBcAg, and wherein the cysteine-modified HBcAg comprises a polynucleotide sequence having at least 70%, or at least 80%, or at least 90%, or at least 95%, or at least 99% homology to SEQ ID NO: 10.

5. The vector of claim 3, wherein the hepatitis B surface antigen (HBsAg) is selected from middle (M), large (L), and small(S) hepatitis B surface antigens.

6. The vector of claim 3, wherein the polymerase (Pol) comprises a truncated and modified polynucleotide sequence, and wherein the polymerase (Pol) comprises a polynucleotide sequence having at least 70%, or at least 80%, or at least 90%, or at least 95%, or at least 99% homology to SEQ ID NO: 12.

7. The vector of claim 1, wherein the heterologous secretion signal sequence is a human IgK secretion signal sequence or a VSV G secretion signal sequence, and wherein the human IgK secretion signal sequence comprises a polynucleotide sequence having at least 70%, or at least 80%, or at least 90%, or at least 95%, or at least 99% homology to SEQ ID NO: 8, wherein the VSV G secretion signal sequence comprises a polynucleotide sequence having at least 70%, or at least 80%, or at least 90%, or at least 95%, or at least 99% homology to SEQ ID NO: 6.

8. The vector of claim 1, wherein the sequence domain encoding an HBV antigen is a cysteine-modified hepatitis B core antigen (HBcAg) comprising a polynucleotide sequence having at least 70% homology to SEQ ID NO: 10, and wherein the heterologous secretion signal sequence is a VSV G secretion signal sequence comprising a polynucleotide sequence having at least 70% homology to SEQ ID NO: 6, or wherein the sequence domain encoding an HBV antigen is a polymerase (Pol) gene comprising a polynucleotide sequence having at least 70% homology to SEQ ID NO: 12, and wherein the heterologous secretion signal sequence is a human IgK secretion signal sequence comprising a polynucleotide sequence having at least 70% homology to SEQ ID NO: 6.

9. The vector of claim 1, wherein the sequence domain encoding a human short hairpin RNA (shRNA) targets PD-L1, and wherein the sequence domain encoding the shRNA comprises a polynucleotide sequence having at least 70%, or at least 80%, or at least 90%, or at least 95%, or at least 99% homology to SEQ. ID NO: 13.

10. The vector of claim 1, wherein the DNA sequence encoding a vesiculovirus glycoprotein encodes a New Jersey (NJ) serotype vesiculovirus glycoprotein, and wherein the NJ serotype vesiculovirus glycoprotein comprises a polynucleotide sequence having at least 70%, or at least 80%, or at least 90%, or at least 95%, or at least 99% homology to SEQ ID NO: 15.

11. The vector of claim 1, wherein the sequence domain encoding an HBV antigen is linked to the sequence encoding a vesiculovirus glycoprotein by a sequence comprising a 2A ribosome skipping sequence, wherein the 2A ribosome skipping sequence is a Thosea asigna virus 2A (T2A) sequence, and, wherein the T2A sequence comprises a polynucleotide sequence having at least 70%, or at least 80%, or at least 90%, or at least 95%, or at least 99% homology to SEQ ID NO: 4.

12. A high-titer hybrid virus vector for treatment, prophylaxis or prevention of hepatitis B virus infections comprising the following operably linked sequence elements:
a) a first DNA sequence comprising a DNA promoter sequence,
b) a second DNA sequence encoding alphavirus non-structural protein polynucleotide sequences and having at least 90% homology to SEQ ID NO: 2;
c) a third DNA sequence encoding at least two alphavirus subgenomic promoters,
d) a fourth DNA sequence comprising at least two sequence domains each independently selected from the group consisting of
i) a sequence domain encoding an HBV antigen comprising a polynucleotide sequence having at least 90% homology to SEQ ID NO: 10 or SEQ ID NO: 12, wherein the sequence domain comprises at least one heterologous secretion signal sequence having at least 90% homology to SEQ ID NO: 6 or SEQ ID NO: 8; and
ii) a sequence domain encoding a human short hairpin RNA (shRNA), the sequence domain comprising a polynucleotide sequence having at least 90% homology to SEQ ID NO: 13; and
e) a fifth DNA sequence encoding a vesiculovirus glycoprotein having at least 90% homology to SEQ ID NO: 15,
optionally wherein the sequence domain encoding an HBV antigen is linked to the sequence encoding a vesiculovirus glycoprotein by a 2A polynucleotide having at least 90% homology to SEQ ID NO: 4.

13. The vector of claim 1, wherein titers of at least 1×1010 plaque forming units (pfu) per mL of virus like vesicles (VLVs) are obtained.

14. Virus-like vesicles (VLVs) containing replicon RNA generated by the high-titer hybrid-virus vector of claim 1.

15. A pharmaceutical composition comprising the virus-like vesicles (VLVs) of claim 14, and a pharmaceutically acceptable carrier.

16. The vector of claim 1, wherein the vector is a plasmid comprising a polynucleotide sequence having at least 70%, or at least 80%, or at least 90%, or at least 95%, or at least 99% homology to SEQ ID NO: 16 or SEQ ID NO: 17.

17. The vector of claim 1, wherein the vector is a plasmid comprising a polynucleotide sequence consisting of SEQ ID NO: 16 or SEQ ID NO: 17.

18. An isolated plasmid comprising a polynucleotide sequence having at least 90% homology to SEQ ID NO: 16 or SEQ ID NO: 17.

19. A therapeutic method comprising administering a therapeutically effective amount of the composition of claim 15 to a mammalian subject in need thereof, wherein the mammalian subject is a human or animal, and wherein said method:
a. treats or prevents hepatitis B virus infections in the mammalian subject,
b. immunizes the mammalian subject against hepatitis B virus infections, and/or
c. downregulates genes associated with hepatitis B virus infections.

20. A method of producing virus-like vesicles (VLVs) for treatment, prophylaxis, or prevention of hepatitis B virus infections comprising the steps of:
a) generating a high-titer virus vector comprising at least two alphavirus sub-genomic promoters; and at least two sequence domains each independently selected from the group consisting of sequence domain encoding HBV antigens: a core (HBcAg), surface [middle (M), large (L), and small(S) HBs], polymerase (Pol) and HBx and combinations thereof, wherein the protein nucleotide sequences comprise at least one heterologous secretion signal sequence; and a sequence domain encoding a human short hairpin RNA (shRNA).
b) transfecting BHK-21 or HEK293 T cells with the high-titer virus vector of step (a),
c) incubating the transfected BHK-21 or HEK293 T cells of step (b) in a buffer solution for a suitable time and at a suitable temperature to propagate VLVs; and
d) isolating the VLVs from the BHK-21 or HEK293 T cells and buffer solution by a technique selected from the group consisting of ultrafiltration, centrifugation, tangential flow filtration, affinity purification, ion exchange chromatography, and combinations thereof;
wherein the isolating of step (d) yields VLVs of a high titer.
